(19) United States
(12) Patent Application Publication
(10) Pub. No.: US 2026/0234667 A1
(43) Pub. Date: Aug. 13, 2026

(54) Nucleic Acids Encoding TGF-Beta Inhibitor and IL-12 and Uses Thereof

(71) Applicant: KaliVir Immunotherapeutics, Inc., Pittsburgh, PA (US)

(72) Inventors: Stephen Howard THORNE, Pittsburgh, PA (US); Mingrui ZHANG, Pittsburgh, PA (US)

(21) Appl. No.: 19/181,919

(22) Filed: Apr. 17, 2025

Related U.S. Application Data
(63) Continuation of application No. PCT/US2023/077151, filed on Oct. 18, 2023.
(60) Provisional application No. 63/417,487, filed on Oct. 19, 2022.
(60) Provisional application No. 63/471,811, filed on Jun. 8, 2023.

(51) Int. Cl. C12N 15/863; A61K 9/00; A61K 31/7088; A61P 35/00; C07K 14/495; C07K 14/54; C07K 14/715

(52) U.S. Cl. CPC C12N 15/8636; A61K 9/0019; A61K 31/7088; A61P 35/00; C07K 14/495; C07K 14/5434; C07K 14/7158; C07K 2319/02; C12N 2710/24143; C12N 2830/34

(57) ABSTRACT

The present disclosure provides for a nucleic acids encoding for a transforming growth factor inhibitor and IL-12. Further provided herein are nucleic acids encoding for chemokine receptor CXCR3. Additionally described herein are oncolytic viruses comprising the nucleic acids described herein. Compositions described herein are further described for use in the treatment of cancer.

### CROSS-REFERENCE

This application is a continuation application of PCT/US2023/077151, filed Oct. 18, 2023, which claims the benefit of U.S. Provisional Application No. 63/417,487 filed Oct. 19, 2022, and U.S. Provisional Application No. 63/471,811 filed Jun. 8, 2023, all of which are incorporated by reference herein in their entirety.

### SEQUENCE LISTING

The instant application contains a Sequence Listing which has been submitted electronically in ST.26 xml format and is hereby incorporated by reference in its entirety. Said xml copy, created on Sep. 28, 2023, is named 199249-720601_SL.xml and is 110,350 bytes in size.

### BACKGROUND

Cancer is a major source of illness, especially in developed countries. Transforming growth factor beta (“TGF-β,” “TGF-beta,” or “TGF-b”) is highly overexpressed by many cancer cell types and provides a means of evading the host immune response. Thus, there is a need for compositions and methods for targeting and modulating TGF-b activity in the microenvironment.

### BRIEF SUMMARY

Described herein are compositions, wherein the compositions comprise: a vector, wherein the vector comprises: an exogenous nucleic acid comprising a sequence encoding for a cytokine or a functional variant thereof; an exogenous nucleic acid comprising a sequence encoding for a chemokine receptor or a functional variant thereof; and a first promoter region, wherein the first promoter region is upstream to the sequence encoding for the chemokine receptor and provides for expression of the chemokine receptor prior to expression of the cytokine.

Described herein are nucleic acids, wherein the nucleic acid comprises a sequence encoding for at least two polypeptides, wherein the at least two polypeptides comprise: interleukin-12 (IL-12) or a functional variant thereof; and a Transforming growth factor beta (TGF-beta) activity inhibitor.

Described herein are nucleic acids, wherein the nucleic acid comprises: a first region encoding a first polypeptide comprising a sequence having at least 85%, 90%, 95%, or 99% sequence identity to SEQ ID NO: 12 or SEQ ID NO: 15; and a second region encoding a second polypeptide comprising a sequence having at least 85%, 90%, 95%, or 99% sequence identity to SEQ ID NO: 40 or SEQ ID NO: 41.

Described herein are nucleic acids, wherein the nucleic acid molecule comprises: an A52R locus comprising, in 5′ to 3′ order: a first promoter region, wherein the promoter comprises an A52R promoter; an insertion of a first region encoding human CXCR3; an insertion at a TK gene locus comprising, in 5′ to 3′ order: a second promoter region, wherein the promoter comprises P135; a second region encoding human IL-12; a third promoter region, wherein the promoter comprises P7.5; and a third region encoding a TGF beta variant.

Described herein are nucleic acids, wherein the nucleic acid molecule comprises: an insertion at an A52R locus comprising a sequence as set forth in SEQ ID NO: 88; an insertion at a TK gene locus comprising a sequence as set forth in SEQ ID NO: 85.

Described herein are pharmaceutical compositions, wherein the pharmaceutical composition comprises: a nucleic acid as described herein or a vector as described herein; and a pharmaceutically acceptable excipient.

Described herein are methods for treatment of cancer comprising administering to a subject having cancer a pharmaceutical composition as described herein in an amount sufficient for treatment of a cancer.

Described herein are methods for activating an anti-tumor immune response, comprising administering to a subject having a cancer a pharmaceutical composition as described herein.

Described herein are methods for reduction of incidence of tumor cell growth, comprising: administering to tumor cells a pharmaceutical composition as described herein in an effective amount sufficient for reduction of incidence of tumor cell growth.

### BRIEF DESCRIPTION OF THE DRAWINGS

The novel features of the disclosure are set forth with particularity in the appended claims. A better understanding of the features and advantages of this disclosure will be obtained by reference to the following detailed description that sets forth illustrative embodiments, in which the principles of this disclosure are utilized, and the accompanying drawings of which:

FIGS. 1A and 1B show the interaction of TGF-b receptor subunits I (RI) and II (RII) to native and modified TGF-beta ligands. FIG. 1A illustrates binding of unmodified TGF-b to RII and RI to generate the receptor complex. FIG. 1B shows binding of a modified TGF-b as described herein, to RII, without a binding capacity to RI, preventing formation of the receptor complex.

FIG. 2 illustrates a diagram of an expression construct having a P7.5 promoter driving expression of IL-12 polypeptide, comprising IL-12 beta and alpha subunits linked by a 22-residue glycine-rich linker and a P28 promoter driving expression of the TGFbf polypeptide, comprising a signal peptide of Interleukin 2 (IL-2sig) fused to a modified TGF-beta variant with cysteines 8 and 17 mutated to valine and arginine, respectively.

FIG. 3A is a line graph showing average volume of induced Renca cell tumors on the y-axis over days post treatment on the x-axis in mice following treatment with buffer control, TK-control, or virus expressing IL-12, TGFbf, or both IL-12 and TGFbf.

FIG. 3B is a line graph showing average volume of induced B16 cell tumors on the y-axis over days post treatment on the x-axis in mice following treatment with buffer control, TK-control, or virus expressing murine IL-12, TGFbf, or both IL-12 and TGFbf.

FIG. 4A is a line graph showing probability of survival on the y-axis over time in days on the x-axis in mice with induced Renca cell tumors treated with PBS (1); TK-control (2); or virus expressing murine IL-12 (3), both murine IL-12 and TGFbf1 (4), or TGFbf1 (5).

FIG. 4B is a line graph showing probability of survival on the y-axis over days on the x-axis in mice with induced B16 cell tumors treated with PBS (1); TK-control (2); or virus expressing murine IL-12 (3), both murine IL-12 and TGFbf1 (4), or TGFbf1 (5).

FIG. 5 illustrates a diagram of an expression construct having a A52R promoter driving expression of CXCR3.

FIG. 6 illustrates a diagram of an expression construct having a 135 promoter driving expression of IL-12 polypeptide, comprising IL-12 beta and alpha subunits linked by a 22-residue glycine-rich linker and a P7.5 promoter driving expression of a TGF-beta variant, TGF-bv2.

FIG. 7 is a diagram showing insertion of a sequence expressing CXCR3 in the A52R locus and sequences expressing IL-12 and a TGF-beta inhibitor in the Tyrosine Kinase locus.

FIG. 8A is a 1-dimensional histogram from a FACS analysis of Hela cells infected with vaccinia virus modified to express CXCR3, IL-12, and TGF-beta inhibitor (TGFBi/IL-12/CXCR3); infected with control virus (CTRL); or no infection (−), indicating increased detection of expressed CXCR3 in Hela cells infected with modified virus.

FIGS. 8B-8E are barplots showing migration of indicated populations of peripheral blood monocytes (PBMC) to CXCR3 ligand CXCL11 after infection with vaccinia virus modified to express CXCR3, IL-12, and TGF-beta inhibitor (TGFBi/IL-12/CXCR3); infected with control virus (CTRL); or no infection (−). FIG. 8B illustrates migration of CD4 cells. FIG. 8C illustrates migration of CD8 cells. FIG. 8D illustrates migration of monocytes. FIG. 8E illustrates migration of B cells.

FIG. 8F is a barplot depicting quantitative ELISA detection of IL-12 in Hela supernatants from cells infected with vaccinia virus modified to express CXCR3, IL-12, and TGF-beta inhibitor; infected with control virus; or no infection, showing expression only detectable in supernatant from Hela cells infected with modified virus.

FIG. 8G is a photo of a Western Blot from Hela lysates infected with vaccinia virus modified to express CXCR3, IL-12, and TGF-beta inhibitor; infected with control virus; or no infection, showing a 12 kDa band corresponding to the TFG-beta inhibitor only detectable in the lysate from cells infected with the modified virus.

FIG. 9A displays traces of fluorescence incorporation in generations of cells, as represented by peaks, in CD8 T cells infected with vaccinia virus modified to express CXCR3, IL-12, and TGF-beta inhibitor (TGFBi/IL-12/CXCR3); infected with control virus (CTRL); or no infection (−) and treated with 0 ng/ml, 10 ng/ml, or 50 ng/ml TGF-beta 1, indicating infection with modified virus inhibits suppression by TGF-beta.

FIG. 9B provides FACS analysis plots of CD8 cells screened for CD44 and granzyme B (GZMB) after infection with vaccinia virus modified to express CXCR3, IL-12, and TGF-beta inhibitor (TGFBi/IL-12/CXCR3); infected with TK-control virus (CTRL); or no infection (−), indicating increased expression of GZMB in cells infected with modified virus.

FIG. 10A is a barplot showing detected PFU/ml in human lung adenocarcinoma (A549) cells, human cervical cancer (Hela) cells, and Human Foreskin Fibroblasts (HFF) following infection with vaccinia virus modified to express CXCR3, IL-12, and TGF-beta inhibitor, indicating increased virus in Hela cells.

FIG. 10B is a plot showing detected copies of viral genome/mg tumor in RENCA tumors following infection with vaccinia virus modified to express CXCR3, IL-12, and TGF-beta inhibitor compared to unmodified virus, showing increased viral counts in tumors infected with modified virus.

FIGS. 11A and 11B are plots of tumor size over 51 days in EMT6 (FIG. 11A) and MC38 (FIG. 11B) tumor models infected with vaccinia virus modified to express CXCR3, IL-12, and TGF-beta inhibitor (TGFBi/IL-12/CXCR3); infected with control virus (CTRL); or no infection (−), showing almost complete suppression of growth in tumors infected with modified virus.

FIGS. 11C and 11D are plots of probability of survival in EMT6 (FIG. 11C) and MC38 (FIG. 11D) tumor models infected with vaccinia virus modified to express CXCR3, IL-12, and TGF-beta inhibitor (TGFBi/IL-12/CXCR3); infected with TK-control virus; or no infection (−), showing increased survival in tumors infected with modified virus.

FIG. 12A is photos of prepared RENCA and MC38 tumor samples treated with CD3, CD8, and nuclear stains after infection with vaccinia virus modified to express CXCR3, IL-12, and TGF-beta inhibitor (TGFBi/IL-12/CXCR3); infected with TK-control virus (CTRL); or no infection (−), showing increased infiltration of CD3 and CD8 cells into tumors after infection with modified virus.

FIGS. 12B-12E are barplots of total counts of CD3+ and CD8+ T cells in RENCA and MC38 tumor samples after infection with vaccinia virus modified to express CXCR3, IL-12, and TGF-beta inhibitor (TGFBi/IL-12/CXCR3); infected with TK-control virus (CTRL); or no infection (−). FIG. 12B shows increased CD3+ cells in RENCA tumors infected with modified virus. FIG. 12C shows increased CD8+ cells in RENCA tumors infected with modified virus. FIG. 12D shows increased CD3+ cells in MC38 tumors infected with modified virus. FIG. 12E shows increased CD8+ cells in MC38 tumors infected with modified virus.

FIG. 13A is a heatmap showing relative expression levels of Type II interferon gamma (INFG)-associated genes compared to an overall mean, in cells treated with vaccinia virus modified to express CXCR3, IL-12, and TGF-beta inhibitor and control cells, showing an increased expression of INFG-associated genes in cells treated with modified virus.

FIG. 13B is a heatmap showing relative expression levels of TGF-beta1-associated genes compared to an overall mean in cells treated with vaccinia virus modified to express CXCR3, IL-12, and TGF-beta inhibitor and control cells, showing an increased expression in TGF-beta1-associated genes in cells treated with modified virus.

### DETAILED DESCRIPTION

TGF-beta1 (TGFB1)-mediated immune resistance is one of the major mechanisms of immune suppression utilized across multiple tumor types. Immune resistance imparted by TGFB1 can be mediated through its pleiotropic effects on vasculature, fibrogenesis and regulatory/effector immune cells within the tumor microenvironment. Blockade of TGFB1 with a TGF-beta inhibitor (TGFBi) can improve response to immunotherapy. Further, IL-12 is a cytokine that, through IFNg induction, can promote type 1 inflammatory response, M1 macrophage skewing, and effector CD8 T cell response. Combining TGFB1 blockade with IL-12 can enhance therapeutic benefits through simultaneously reducing immunosuppression and enhancing anti-tumor immune response. In addition, CXCR3 expression from the viral backbone can enhance systemic virus delivery to CXCR3 ligand-rich tumors.

Current TGF-beta inhibitors have challenges in specificity and delivery. For example, receptor-targeting small molecule receptor kinase inhibitors (SMRKIs) are prone to poor specificity and selectivity. Antibodies and receptor traps are reported to penetrate poorly into dense tissues, such as tumors. As a solution to these issues TGF-beta variants provided herein offer specific receptor targeting and improved tissue penetration. The small size of the polypeptide allows for increased tissue penetration. Moreover, as described herein, delivery can be further enhanced by providing a nucleic acid encoding for the TGF-beta inhibitor in a delivery vector. In addition, as described herein, delivery of TGF-beta inhibitor in combination with IL-12 showed greater overall response and survival.

Provided herein are compositions and methods for a vaccinia-based immunotherapy, combining enhanced systemic virus delivery to CXCR3 ligand rich tumors and locally expressed IL-12 and TGFBi within the tumor microenvironment. In some embodiments, methods comprise a treatment of cancer. Compositions described herein can comprise one or more nucleic acids encoding for polypeptides as described herein. Nucleic acids provided herein can comprise DNA, RNA, nucleic acid analogues, or any combination thereof. Briefly, described herein are (1) compositions for expression of a TGFb inhibitor and IL-12, (2) combinations of nucleic acids, (3) compositions for expression of chemokine receptors (4) vectors for expression of modified nucleic acids, (5) modified oncolytic viruses, (6) conditions for treatment, and (7) dosage amounts, forms, and methods of administration of compositions described herein.

### Definitions

The terminology used herein is for the purpose of describing particular cases only and is not intended to be limiting. As used herein, the singular forms “a”, “an” and “the” include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, e.g., the limitations of the measurement system. Where particular values are described in the application and claims, unless otherwise stated the term “about” should be assumed to mean an acceptable error range for the particular value, such as ±10% of the value modified by the term “about”.

The terms “heterologous nucleic acid sequence,” or “exogenous nucleic acid sequence,” or “transgenes,” as used herein, in relation to a specific virus can refer to a nucleic acid sequence that originates from a source other than the specified virus.

The term “mutation,” as used herein, can refer to a deletion, an insertion of a heterologous nucleic acid, an inversion, or a substitution, including an open reading frame ablating mutations as commonly understood in the art.

The term “gene,” as used herein, can refer to a segment of nucleic acid that encodes for an individual protein or RNA (also referred to as a “coding sequence” or “coding region”), optionally together with associated regulatory regions such as promoters, operators, terminators, and the like, which may be located upstream or downstream of the coding sequence.

A “promoter,” as used herein, can be a control sequence that is a region of a nucleic acid sequence at which initiation and rate of transcription are controlled. In certain embodiments, a promoter may contain genetic elements at which regulatory proteins and molecules may bind such as RNA polymerase and other transcription factors. The terms “operatively positioned,” “operatively linked,” “under control” and “under transcriptional control” can mean that a promoter is in a correct functional location and/or orientation in relation to a nucleic acid sequence to control transcriptional initiation and/or expression of that sequence. In certain embodiments, a promoter may or may not be used in conjunction with an “enhancer,” which refers to a cis-acting regulatory sequence involved in the transcriptional activation of a nucleic acid sequence.

The term “homology,” as used herein, may be to calculations of “homology” or “percent homology” between two or more nucleic acid or amino acid sequences that can be determined by aligning the sequences for optimal comparison purposes (e.g., gaps can be introduced in the sequence of a first sequence). The nucleic acids at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleic acid as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The homology can be between the entire lengths of two sequences or between fractions of the entire lengths of two sequences. The two sequences can be genes, nucleic acid sequences, protein sequences, peptide sequences, amino acid sequences, or fragments thereof. The actual comparison of the two sequences can be accomplished by well-known methods, for example, using a mathematical algorithm. When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA.

The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” can be meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” can refer to the amount of a compound that, when administered, can be sufficient to prevent development of, or alleviate to some extent, one or more of the symptoms of the disorder, disease, or condition being treated.

The term “oncolytic,” as used herein, can refer to killing of cancer or tumor cells by an agent, such as an oncolytic poxvirus, such as an oncolytic vaccinia virus, e.g., through the direct lysis of said cells, by stimulating immune response towards said cells, apoptosis, expression of toxic proteins, autophagy and shutdown of protein synthesis, induction of anti-tumoral immunity, or any combinations thereof. The direct lysis of the cancer or tumor cells infected by the agent, such as an oncolytic vaccinia virus, can be a result of replication of the virus within said cells. In certain examples, the term “oncolytic,” can refer to killing of cancer or tumor cells without lysis of said cells.

The term “oncolytic virus” as used herein can refer to a virus that preferentially infects and kills tumor cells. In some embodiments, the oncolytic viruses can include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use. In some embodiments, the oncolytic virus can be a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a lentivirus, a mengovirus, or a myxoma virus. In certain embodiments, the oncolytic virus can be a poxvirus. In certain embodiments, the oncolytic virus can be a vaccinia virus.

The term “modified oncolytic virus” as used herein can refer to an oncolytic virus that comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of an exogenous protein or modified viral protein to the viral capsid. In general, oncolytic viruses may be modified (also known as “engineered”) in order to gain improved therapeutic effects against tumor cells. In some embodiments, the modified oncolytic virus can be a modified poxvirus. In some embodiments, the modified oncolytic virus can be a modified poxvirus. In some embodiments, the modified oncolytic virus can be a modified vaccinia virus.

The terms “systemic delivery,” and “systemic administration,” used interchangeably herein, in some cases can refer to a route of administration of medication, oncolytic virus or other substances into the circulatory system. The systemic administration may comprise oral administration, intraperitoneal administration, parenteral administration, intranasal administration, sublingual administration, rectal administration, transdermal administration, intra-arterial administration, or any combinations thereof.

### TGF-Beta Activity Inhibitors

The cytokine TGF-beta in each of its isoforms, TGF-beta1, TGF-beta2, and TGF-beta3 is a potent suppressor of immunity. TGF-beta have been reported to suppress the proliferation of cytotoxic T-lymphocytes (CTLs), natural killer (NK) cells, and dendritic cells (DCs). The cytokine also stimulates the proliferation and activation of regulatory T-cells (Treg).

Provided herein are TGF-beta activity inhibitors for treatment of cancer and related symptoms. TGF-beta signaling activity affects the progression of many diseases, including cancers. TGF-beta assembles the receptors TGF-beta RI and TGF-beta RII in to a signaling tetramer by binding TGF-beta RII then recruiting TGF-beta RI. Preventing binding to, or assembly of, the receptors blocks the TGF-beta signaling cascade. Inhibitors are provided herein that prevent recruitment of TGF-beta RI.

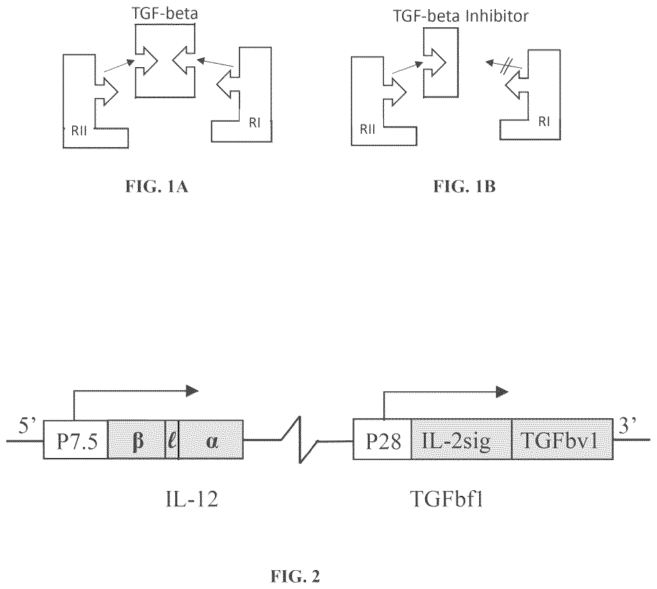

Provided herein are compositions comprising nucleic acids encoding for TGF-beta activity inhibitors. In some embodiments, the TGF-beta inhibitors are capable of binding to TGF beta receptor II and antagonizing expression of TGF-beta (FIG. 1B). Without being bound by theory, in some embodiments, the TGF-beta inhibitor specifically targets the cognate receptor, antagonizing the receptor. In further embodiments, the TGF-beta inhibitor has less adverse effects associated with off-target activity. A nucleic acid expressing a TGF-beta inhibitor as described herein can provide increased tissue penetration. In some embodiments described herein, the TGF-beta inhibitor binds the TGFb receptor II. In some embodiments, the TGF-beta inhibitor does not bind TGFb receptor I.

Native TGF-beta is a dimer of two identical 112-residue peptides connected by a disulfide bond. In some embodiments, a nucleic acid described herein encodes for human TGF-beta monomer or any of three isoforms, TGF-beta 1, TGF-beta 2, or TGF-beta 3, as described by SEQ ID NOs: 1-3 in Table 1, or a functional variant thereof. In some embodiments, a nucleic acid described herein encodes for engineered mini monomers of TGF-beta 1, TGF-beta 2, and TGF-beta 3, as described by SEQ ID NOs: 4, 5, and 9, or a functional variant thereof. In some embodiments, engineered mini monomers of TGF-beta 2 described herein include deletion of an alpha 3 helix at residues 52-71 and substitution of Cys-77 with serine, with amino acid numbering based on SEQ ID NO: 2. In some embodiments, engineered mini monomer of TGF-beta 2 includes modifications to include the cystine-knot region of protein related to Dan and Cerberus (PRDC). In some, embodiments, engineered mini monomers of TGF-beta 2 lacks the alpha-3 heel helix of native TGF-beta. Exemplary sequences for inclusion in compositions described herein are listed in Table 1, SEQ ID NOs: 4-9.

Exemplary variants of the TGF-beta 2 mini monomer are described by SEQ ID NOs: 6, 7, and 8. Further provided herein are nucleic acids encoding for mini monomer variants comprising substitutions allowing for increased solubility and higher binding affinity.

TABLE 1
Sequences of TGF-beta isoforms, monomers, and mini monomers

| SEQ ID NO: | Isoform/variant | Amino Acid Sequence |
|---|---|---|
| 1 | TGF-beta 1 | MALDTNYCFSSTEKNCCVRQLYIDFRKDLGWKWIHEPKGYHANF |
| | | CLGPCPYIWSLDTQYSKVLALYNQHNPGASAAPCCVPQALEPLPI |
| | | VYYVGRKPKVEQLSNMIVRSCKCS |
| 2 | TGF-beta 2 | MALDAAYCFRNVQDNCCLRPLYIDFKRDLGWKWIHEPKGYNAN |
| | | FCAGACPYLWSSDTQHTKVLSLYNTINPEASASPCCVSQDLEPLTI |
| | | LYYIGKTPKIEQLSNMIVKSCKCS |
| 3 | TGF-beta 3 | MALDTNYCFRNLEENCCVRPLYIDFRQDLGWKWVHEPKGYYAN |
| | | FCSGPCPYLRSADTTHSTVLGLYNTLNPEASASPCCVPQDLEPLTI |
| | | LYYVGRTPKVEQLSNMVVKSCKCS |
| 4 | TGF-beta 1 | MALDTNYCFSSTEKNCCVRQLYIDFRKDLGWKWIHEPKGYHANF |
| | mini | CLGPCPYRASKSPSCVPQALEPLPIVYYVGRKPKVEQLSNMIVRSC |
| | monomer | KCS |
| 5 | TGF-beta 2 | MALDAAYCFRNVQDNCCLRPLYIDFKRDLGWKWIHEPKGYNAN |
| | mini | FCAGACPYRASKSPSCVSQDLEPLTILYYIGKTPKIEQLSNMIVKSC |
| | monomer | KCS |
| 6 | TGF-beta 2 | FALDAAYCFRNVQDNCCLRPLYIDFRKDLGWKWIHEPKGYNANF |
| | mini monomer | CAGACPYRASKSPSCVSQDLEPLTIVYYVGRKPKVEQLSNMIVKS |
| | variant A | CKCS |
| 7 | TGF-beta 2 | ALDAAYVFRNVQDNCALRPLYIDFRKDLGWKWIHEPKGYNANF |
| | variant B | CAGACPYRASKSPRCRSQDLEPLTIVYYVGRKPKVEQLSNMIVKS |
| | (TGFbv1) | CKCS |
| 8 | TGF-beta 2 | KEVLASSQEALVVTERKYLKSDWCKLRPLYIDFRKDLGWKWIHE |
| | variant mP | PKGYNANFCYGQCNSFYIPRHVKKEEDSFQSSAFCVSQDLEPLTIV |
| | (TGFbv2) | YYVGRKPKVEQLSNMIVKSCRCMSV |
| 9 | TGF-beta 3 | MALDTNYCFRNLEENCCVRPLYIDFRQDLGWKWVHEPKGYYAN |
| | mini | FCSGPCPYEESDSPSCVPQDLEPLTILYYVGRTPKVEQLSNMVVKS |
| | monomer | CKCS |

In some embodiments, a nucleic acid described herein encodes for a TGF-beta inhibitor comprising an antibody or a functional fragment thereof. In some embodiments, a nucleic acid described herein encodes for an antibody or functional fragment thereof binds TGF-beta. Non-limiting examples of antibodies binding TGF-beta include 2G7, 1D11, GC1008, LY2382770, and TbetaM1. In some embodiments, a nucleic acid described herein encodes for an antibody or functional fragment thereof that binds a TGF-beta receptor. In some embodiments the antibody or functional fragment thereof binds TGF-beta receptor I, TGF-beta receptor II, or TGF-beta receptor III.

In some embodiments, a nucleic acid described herein encodes for a TGF-beta inhibitor comprising a receptor trap. In some embodiments, the encoded receptor trap blocks the entire receptor binding interface. In some embodiments, the encoded receptor trap is an affinity-optimized, soluble variant of the extracellular binding domain. In some embodiments, the target ligand preferentially binds the receptor trap. In some embodiments, the encoded receptor trap prevents ligand binding to the receptor. In some embodiments, the encoded TGF-beta inhibitor controls signaling with the receptor.

In some embodiments, a nucleic acid described herein encodes for a TGF-beta inhibitor comprising a peptide. The peptide, in non-exclusive embodiments, comprises P144 or P17. In selected embodiments, a nucleic acid described herein encodes for a TGF-beta inhibitor comprising a sequence as described in Table 2. In some embodiments, a nucleic acid described herein encodes for a TGF-beta inhibitor peptide described by SEQ ID NO: 10 or 11. In some embodiments, a nucleic acid described herein encodes for a TGF-beta inhibitor that binds TGF-beta. In further embodiments, the TGF-beta-TGF-beta inhibitor complex does not bind to a TGF-beta receptor. Said peptides optionally bind isoforms of TGF-beta.

TABLE 2
Inhibitory peptide sequences.

| SEQ ID NO: | Peptide | Amino Acid Sequence |
|---|---|---|
| 10 | P17 | KRIWFIPRSSWYERA |
| 11 | P144 | TSLDASIIWAMMQN |

In some embodiments, a nucleic acid described herein encodes for a TGF-beta inhibitor comprising a dominant-negative receptor. The dominant-negative receptor, in some embodiments, comprises a truncated TGF-beta receptor I, receptor II, or receptor III. In some embodiments, a nucleic acid described herein encodes for a dominant-negative receptor comprising a truncated TGF-beta receptor II. In some embodiments, the dominant-negative receptor is soluble. In some embodiments, the encoded dominant-negative receptor is version of TGF-beta receptor I, receptor II, or receptor III lacking a transmembrane region.

In some embodiments, a nucleic acid described herein encodes for a TGF-beta inhibitor comprising a dominant-negative inhibitor. The encoded dominant-negative inhibitor, in some embodiments, binds to TGF-beta receptor I, receptor II, or receptor III. In some embodiments, the encoded dominant-negative inhibitor binds to TGF-beta receptor II. In some embodiments, the encoded dominant-negative inhibitor prevents TGF-beta inhibitors optionally include variants of TGF-beta 1, TGF-beta 2, or TGF-beta 3. Non-exclusive embodiments encoded by nucleic acids described herein bind the ligand binding domain of the TGF-beta receptor II.

Provided here are compositions comprising nucleic acids encoding for a TGF-beta 2 variant (TGFbv1). In some embodiments, the nucleic acid sequence encodes for a peptide described by SEQ ID NO: 7. In some embodiments, the encoded TGFbv1 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 7.

Provided here are compositions comprising nucleic acids encoding for a TGF-beta 2 variant (TGFbv2). In some embodiments, the nucleic acid sequence encodes for a peptide described by SEQ ID NO: 8. In some embodiments, the encoded TGFbv2 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 8.

TGF-beta can suppress proliferation of stimulated immune cells, such as T cells, below a baseline level. Compositions comprising a TGF-beta inhibitor described herein can counteract suppression by TGF-beta and can allow stimulated immune cells to multiply at a level approaching the baseline. In some embodiments, contacting TGF-beta-suppressed cells with a TGF-beta inhibitor as described herein can provide for proliferation at about 10%, about 20%, about 30%, about 40%, about 50%, about 60%, about 70%, about 80%, about 90%, or about 100% of a baseline in unsuppressed cells.

A TGF-beta inhibitor can modulate expression in a family of associated genes. In some embodiments, a modulation comprises inducing expression of genes. In some embodiments, contacting cells with a TGF-beta inhibitor as described herein induces expression in one or more interferon gamma (IFNG)-associated genes as compared to a median. In some embodiments, the expression of one or more IFNG-associated genes is increased by about 10%, about 20%, about 30%, about 40%, about 50%, about 60%, about 70%, about 80%, about 90%, about 100%, about 120%, %, about 140%, about 160%, about 180%, about 200%, about 250%, about 300%, about 350%, or about 400% compared to a median of expression across treated and untreated cells. IFNG-associated genes are selected from the group consisting of CXCL11, XCR1, STAT1, IDO1, IL12B, IFNG, CIITA, H2-EB1, H2-AB1, TBX21, CXCR3, CD2, LTB, CXCL16, B2M, VCAM1, TAP1, IFIT2, TAP2, IL2RG, STAT2, CD274, and IRF1.

In some embodiments, contacting cells with a TGF-beta inhibitor as described herein reduces expression in TGF-β1-associated genes as compared to a median. In some embodiments, the expression of TGF-β1-associated genes is decreased by about 10%, about 20%, about 30%, about 40%, about 50%, about 60%, about 70%, about 80%, about 90%, or about 100% compared to a median of expression across treated and untreated cells. TGF-β1-associated genes are selected from the group consisting of ILz1B, LPL, SLP1, FBN1, LCN2, CXCL5, OGN, PLOD2, TNFAIP6, CAN, ABCG1, ACKR3, and COL15A1.

### Granzyme B

Granzyme B (GZMB) is found in granules of immune cells such as natural killer cells (NK cells) and cytotoxic T cells. It can be secreted by these cells to mediate apoptosis in target cells. GZMB can also be produced by non-cytotoxic cells such as basophils and mast cells. It can assist in inducing inflammation and extracellular matrix degradation. Activated immune cells can show increased GZMB expression. Compositions comprising a TGF-beta inhibitor described herein can prevent suppression by TGF-beta and allow stimulated immune cells to express GZMB.

Contacting immune cells with a modified virus as described herein can induce expression of granzyme B (GZMB) greater than in untreated cells. In some embodiments, cells contacted with a TGF-beta inhibitor comprise about 10%, about 20%, about 30%, about 40%, about 50%, about 60%, about 70%, about 80%, about 90%, about 100%, about 125%, about 150%, about 200%, about 300%, about 400%, about 500%, about 600%, about 700%, about 800%, about 900%, about 1000% more GZMB than untreated cells.

### IL-12

Provided here are compositions comprising nucleic acids encoding for IL-12 or a functional variant thereof. In some embodiments, a nucleic acid described herein encodes for at least two polypeptides. In some embodiments, the nucleic acid encodes for a first polypeptide comprising an Interleukin 12 or a functional variant thereof. In some embodiments the nucleic acid encodes for an IL-12. In further embodiments, the IL-12 comprises subunit beta (IL-12b) and subunit alpha (IL-12a). In some embodiments, the nucleic acid encodes a murine IL-12 (mIL-12) sequence as described by SEQ ID NO: 12. In some instances, the encoded mIL-12 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 12. In some embodiments, the nucleic acid encodes a human IL-12 (hIL-12) sequence as described by SEQ ID NO: 15. In some instances, the encoded hIL-12 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 15.

Provided here are compositions comprising nucleic acids encoding for a murine IL-12 subunit alpha (IL-12a) (UniProtKB accession ID 43431.1). In some embodiments, the nucleic acid sequence encodes for a peptide described by SEQ ID NO: 13. In some instances, the encoded IL-12a comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 13.

Provided here are compositions comprising nucleic acids encoding for a murine IL-12 subunit beta (IL-12b) (UniProtKB accession ID P43432.1). In some embodiments, the nucleic acid sequence encodes for a peptide described by SEQ ID NO: 14). In some instances, the encoded IL-12b comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 14.

Provided here are compositions comprising nucleic acids encoding for a human IL-12 subunit alpha (hIL-12a) (UniProtKB accession ID P060595). In some embodiments, the nucleic acid sequence encodes for a peptide described by SEQ ID NO: 16. In some instances, the encoded hIL-12a comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 16.

Provided here are compositions comprising nucleic acids encoding for a human IL-12 subunit beta (hIL-12b) (UniProtKB accession ID P29460). In some embodiments, the nucleic acid sequence encodes for a peptide described by SEQ ID NO: 17). In some instances, the encoded IL-12b comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 17.

Exemplary amino acid sequences for IL-12 regions for inclusion in compositions described here are listed in Table 3.

TABLE 3
IL-12 amino acid sequences.

| SEQ | | UniProtKB | |
|---|---|---|---|
| ID NO: | Description | accession ID | Amino Acid Sequence |
| 12 | Murine IL- | | MCPQKLTISWFAIVLLVSPLMAMWELEKDVYV |
| | 12 (mIL-12 | | VEVDWTPDAPGETVNLTCDTPEEDDITWTSDQR |
| | | | HGVIGSGKTLTITVKEFLDAGQYTCHKGGETLS |
| | | | HSHLLLHKKENGIWSTEILKNFKNKTFLKCEAP |
| | | | NYSGRFTCSWLVQRNMDLKFNIKSSSSSPDSRA |
| | | | VTCGMASLSAEKVTLDQRDYEKYSVSCQEDVT |
| | | | CPTAEETLPIELALEARQQNKYENYSTSFFIRDII |
| | | | KPDPPKNLQMKPLKNSQVEVSWEYPDSWSTPH |
| | | | SYFSLKFFVRIQRKKEKMKETEEGCNQKGAFLV |
| | | | EKTSTEVQCKGGNVCVQAQDRYYNSSCSKWA |
| | | | CVPCRVRSGGGGSGGGGSGGGGSGGGGSASAT |
| | | | LALLNHLSLARVIPVSGPARCLSQSRNLLKTTDD |
| | | | MVKTAREKLKHYSCTAEDIDHEDITRDQTSTLK |
| | | | TCLPLELHKNESCLATRETSSTTRGSCLPPQKTS |
| | | | LMMTLCLGSIYEDLKMYQTEFQAINAALQNHN |
| | | | HQQIILDKGMLVAIDELMQSLNHNGETLRQKPP |
| | | | VGEADPYRVKMKLCILLHAFSTRVVTINRVMG |
| | | | YLSSA |
| 13 | Murine IL- | P43431.1 | MCQSRYLLFLATLALLNHLSLARVIPVSGPARC |
| | 12 alpha | | LSQSRNLLKTTDDMVKTAREKLKHYSCTAEDID |
| | (mIL-12a) | | HEDITRDQTSTLKTCLPLELHKNESCLATRETSS |
| | | | TTRGSCLPPQKTSLMMTLCLGSIYEDLKMYQTE |
| | | | FQAINAALQNHNHQQIILDKGMLVAIDELMQSL |
| | | | NHNGETLRQKPPVGEADPYRVKMKLCILLHAFS |
| | | | TRVVTINRVMGYLSSA |
| 14 | Murine IL- | P43432.1 | MCPQKLTISWFAIVLLVSPLMAMWELEKDVYV |
| | 12 beta | | VEVDWTPDAPGETVNLTCDTPEEDDITWTSDQR |
| | (mIL-12b) | | HGVIGSGKTLTITVKEFLDAGQYTCHKGGETLS |
| | | | HSHLLLHKKENGIWSTEILKNFKNKTFLKCEAP |
| | | | NYSGRFTCSWLVQRNMDLKFNIKSSSSSPDSRA |
| | | | VTCGMASLSAEKVTLDQRDYEKYSVSCQEDVT |
| | | | CPTAEETLPIELALEARQQNKYENYSTSFFIRDII |
| | | | KPDPPKNLQMKPLKNSQVEVSWEYPDSWSTPH |
| | | | SYFSLKFFVRIQRKKEKMKETEEGCNQKGAFLV |
| | | | EKTSTEVQCKGGNVCVQAQDRYYNSSCSKWA |
| | | | CVPCRVRS |
| 15 | Human IL- | | MCHQQLVISWFSLVFLASPLVAIWELKKDVYV |
| | 12 (hIL-12) | | VELDWYPDAPGEMVVLTCDTPEEDGITWTLDQ |
| | | | SSEVLGSGKTLTIQVKEFGDAGQYTCHKGGEVL |
| | | | SHSLLLLHKKEDGIWSTDILKDQKEPKNKTFLR |
| | | | CEAKNYSGRFTCWWLTTISTDLTFSVKSSRGSS |
| | | | DPQGVTCGAATLSAERVRGDNKEYEYSVECQE |
| | | | DSACPAAEESLPIEVMVDAVHKLKYENYTSSFFI |
| | | | RDIIKPDPPKNLQLKPLKNSRQVEVSWEYPDTW |
| | | | STPHSYFSLTFCVQVQGKSKREKKDRVFTDKTS |
| | | | ATVICRKNASISVRAQDRYYSSSWSEWASVPCS |
| | | | GGGGGGGGSGGGGSGGGGSASWPPGSASQPP |
| | | | PSPAAATGLHPAARPVSLQCRLSMCPARSLLLV |
| | | | ATLVLLDHLSLARNLPVATPDPGMFPCLHHSQN |
| | | | LLRAVSNMLQKARQTLEFYPCTSEEIDHEDITKD |
| | | | KTSTVEACLPLELTKNESCLNSRETSFITNGSCL |
| | | | ASRKTSFMMALCLSSIYEDLKMYQVEFKTMNA |
| | | | KLLMDPKRQIFLDQNMLAVIDELMQALNFNSET |
| | | | VPQKSSLEEPDFYKTKIKLCILLHAFRIRAVTIDR |
| | | | VMSYLNAS |
| 16 | Human IL- | P29459 | WPPGSASQPPPSPAAATGLHPAARPVSLQCRLS |
| | 12 alpha | | MCPARSLLLVATLVLLDHLSLARNLPVATPDPG |
| | (hIL-12a) | | MFPCLHHSQNLLRAVSNMLQKARQTLEFYPCTS |
| | | | EEIDHEDITKDKTSTVEACLPLELTKNESCLNSR |
| | | | ETSFITNGSCLASRKTSFMMALCLSSIYEDLKMY |
| | | | QVEFKTMNAKLLMDPKRQIFLDQNMLAVIDEL |
| | | | MQALNFNSETVPQKSSLEEPDFYKTKIKLCILLH |
| | | | AFRIRAVTIDRVMSYLNAS |
| 17 | Human IL- | P29460 | MCHQQLVISWFSLVFLASPLVAIWELKKDVYV |
| | 12 beta | | VELDWYPDAPGEMVVLTCDTPEEDGITWTLDQ |
| | (hIL-12b) | | SSEVLGSGKTLTIQVKEFGDAGQYTCHKGGEVL |
| | | | SHSLLLLHKKEDGIWSTDILKDQKEPKNKTFLR |
| | | | CEAKNYSGRFTCWWLTTISTDLTFSVKSSRGSS |
| | | | DPQGVTCGAATLSAERVRGDNKEYEYSVECQE |
| | | | DSACPAAEESLPIEVMVDAVHKLKYENYTSSFFI |
| | | | RDIIKPDPPKNLQLKPLKNSRQVEVSWEYPDTW |
| | | | STPHSYFSLTFCVQVQGKSKREKKDRVFTDKTS |
| | | | ATVICRKNASISVRAQDRYYSSSWSEWASVPCS |

### Linkers and Signaling Domains

Provided herein are compositions comprising a nucleic acid encoding for a linker. In some embodiments, the nucleic acid encoding for the linker is located between various encoded biologically functional units described herein. In some embodiments, the encoded linker is flexible or rigid. In further embodiments, the encoded linker is a cleavable linker. In further embodiments, the encoded cleavable linker comprises a disulfide bond. In further embodiments, the encoded cleavable linker comprises a protease sensitive domain. A non-limiting list of exemplary linkers encoded by nucleic acids comprised in compositions described herein is listed in Table 4. In some embodiments, a composition described herein comprises a nucleic acid encoding for a linker having a sequence as described by SEQ ID NO: 18. In some embodiments, a nucleic acid encodes for a linker comprising at least 70%, at least 75%, at least 80%, at least 85%, at least 90%, at least 95% sequence identity to SEQ ID NO: 18.

TABLE 4
Linkers.

| SEQ ID NO: | Linker type | Amino Acid Sequence |
|---|---|---|
| 18 | Flexible | GGGGSGGGGSGGGGSGGGGSAS |
| 19 | Flexible | (GGGGS)₁₋₃ |
| 20 | Flexible | (Gly)₆₋₈ |
| 21 | Rigid | (EAAAK)₁₋₃ |
| 22 | Rigid | A(EAAAK)₄ALEA(EAAAK)₄A |
| 23 | Rigid | PAPAP |
| | Cleavable | S-S |
| 25 | Cleavable | VSQTSKLTR↓AETVFPDV |
| 26 | Cleavable | PLG↓LWA |
| 27 | Cleavable | RVL↓AEA |
| 28 | Cleavable | EDVVCC↓SMSY |
| 29 | Cleavable | GGIEGR↓GS |
| 30 | Cleavable | TRHRQPR↓GWE |
| 31 | Cleavable | AGNRVRR↓SVG |
| 32 | Cleavable | RRRRRRR↓R↓R |
| 33 | Cleavable | GFLG↓ |
| | Dipeptide | LE |
| 35 | Cleavable | GGGGSSPLGLWAGGGGS |

Subscript in sequence denotes repeats.
Arrow in sequence indicates location of cleavage site.

Provided herein are compositions comprising nucleic acids encoding for signaling domains for controlling cellular functions. The activity of interleukins, for example and without limitation, IL-2 and IL-15, is dependent on processing of a signal peptide. Provided herein are compositions comprising nucleic acids encoding for at least one signal peptide. Table 5 shows non-limiting examples of signal peptides encoded for by nucleic acids in compositions described herein.

TABLE 5
Signal sequences.

| SEQ ID NO: | Signal peptide | Amino Acid Sequence |
|---|---|---|
| 36 | Murine IL-2sig | MYSMQLASCVTLTLVLLVNS |
| 37 | Human IgE | MDWTWILFLVAAATRVHSGR |
| | signal | |
| 38 | IL-15L | MRISKPHLRSISIQCYLCLLLNSHFLTEAGIHVFILGCFSA |
| | | GLPKTEA |
| 39 | IL-15S | MVLGTIDLCSCFSAGLPKTEA |

Provided herein are compositions comprising nucleic acids encoding for a murine IL-2 signal sequence (mIL-2sig) (UniProtKB accession ID P04351.1). In some embodiments, the nucleic acid sequence encodes for the mIL-2sig corresponding to SEQ ID NO: 36 or a functional variant thereof. In some embodiments, the encoded mIL-2sig comprises at least 70%, at least 75%, at least 80%, at least 85%, at least 90%, at least 95% sequence identity to SEQ ID NO: 36.

Provided herein are compositions comprising nucleic acids encoding for a human IgE signal sequence (hIgEsig). In some embodiments, the nucleic acid sequence encodes for the hIgEsig corresponding to SEQ ID NO: 37 or a functional variant thereof. In some embodiments, the encoded hIgEsig comprises at least 70%, at least 75%, at least 80%, at least 85%, at least 90%, at least 95% sequence identity to SEQ ID NO: 37.

### Combination Nucleic Acid Sequences

Provided herein are compositions comprising nucleic acids encoding for a combination of technical features. In some embodiments, a nucleic acid encodes for an IL-12 or functional variant thereof, a signal peptide, and a TGF-beta variant polypeptide. In some embodiments, a nucleic acid encodes for an IL-12, a murine IL-2 signal peptide, and a TGF-beta variant polypeptide or functional variant thereof. In some embodiments, a nucleic acid encodes for an IL-12, a human IgE signal peptide, and a TGF-beta variant polypeptide or functional variant thereof.

In alternative embodiments, two nucleic acids are provided, wherein a first nucleic acid encodes for a first polypeptide comprising an Interleukin 12 or a functional variant thereof, and a second nucleic acid encodes for a second polypeptide comprising a TGF-beta fusion (TGFbf) polypeptide comprising a signal peptide and a TGF-beta variant. In some embodiments, the TGFbf polypeptide comprises the sequences as set forth in SEQ ID NO: 36 and SEQ ID NO: 7 (TGFbf1). In some embodiments, the TGFbf polypeptide comprises the sequences as set forth in SEQ ID NO: 37 and SEQ ID NO: 8 (TGFbf2).

Provided herein are compositions comprising nucleic acid sequences encoding for a polypeptide. In some embodiments, the nucleic acid encodes for a polypeptide comprising a TGFbf1 described by SEQ ID NO: 40 and shown in Table 6. In some embodiments, the encoded TGFbf comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 40. In some embodiments, the nucleic acid encodes for a polypeptide comprising a TGFbf2 described by SEQ ID NO: 41 and shown in Table 6. In some embodiments, the encoded TGFbf comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 41.

TABLE 6
TGF-beta fusion amino acid sequence.

| SEQ ID | | |
|---|---|---|
| NO: | Description | Amino Acid Sequence |
| 40 | TGFbf1 | MYSMQLASCVTLTLVLLVNSALDAAYVFRNVQDNCALRPLYIDF |
| | | RKDLGWKWIHEPKGYNANFCAGACPYRASKSPRCRSQDLEPLTI |
| | | VYYVGRKPKVEQLSNMIVKSCKCS |
| 41 | TGFbf2 | MDWTWILFLVAAATRVHSGRKEVLASSQEALVVTERKYLKSDW |
| | | CKLRPLYIDFRKDLGWKWIHEPKGYNANFCYGQCNSFYIPRHVK |
| | | KEEDSFQSSAFCVSQDLEPLTIVYYVGRKPKVEQLSNMIVKSCRC |
| | | MSV |

### Chemokine Receptors

Chemokines are chemotactic cytokines that regulate the trafficking and positioning of cells by activating the seven-transmembrane spanning chemokine receptors. In some cases, chemokines are divided into four subfamilies based on the position of the first two N-terminal cysteine residues, including the CC, CXC, CX3C and XC subfamilies. Differential expression of chemokine receptors on leukocytes optionally results in selective recruitment of specific cell types under particular conditions, providing appropriate and efficient immune responses tailored to the infecting pathogen or foreign insult. Beyond their pivotal role in the coordinated migration of immune cells to the site of inflammation, in many cases, chemokines also play important roles in the development of lymphoid tissues, in the maturation of immune cells, and in the generation and delivery of adaptive immune responses.

Tumors are increasingly recognized as a complex microenvironment made up of many different cell types that cohabit and communicate with each other in a complicated signaling network. Chemokines are essential coordinators of cellular migration and cell-cell interactions and therefore have great impact on tumor development. In the tumor microenvironment, tumor-associated host cells and cancer cells release an array of different chemokines, resulting in the recruitment and activation of different cell types that mediate the balance between antitumor and pro-tumor responses. In addition to their primary role as chemoattractants, chemokines, in many cases, are also involved in other tumor-related processes, including tumor cell growth, angiogenesis and metastasis.

Tumor cells have been shown to acquire the ability to produce growth-promoting chemokines. For instance, melanoma has been found to express a number of chemokines, including CXCL1, CXCL2, CXCL3, CXCL8, CCL2 and CCL5, which have been implicated in tumor growth and progression. CCL2 level can be found increased in neuroblastoma cell lines and primary tumor cells isolated from human patients. Immunostaining studies also suggest an elevated expression level of CXCL12 in a variety of cancers, including breast cancer, carcinoid, cervical cancer, colorectal cancer, endometrial cancer, liver cancer, lung cancer, lymphoma, melanoma, ovarian cancer, pancreatic cancer, prostate cancer, stomach cancer.

Chemokine receptors are cytokine receptors found on the surface of certain cells that interact with chemokines. There have been 20 distinct chemokine receptors discovered in humans. Each has a 7-transmembrane structure and couples to G-protein for signal transduction within a cell, making them members of a large protein family of G protein-coupled receptors. Following interaction with their specific chemokine ligands, chemokine receptors trigger a flux in intracellular calcium (Ca²⁺) ions (calcium signaling). This causes cell responses, including the onset of a process known as chemotaxis that traffics the cell to a desired location within the organism. In general, the term “chemokine receptor” as used herein can refer to a membrane associated protein that selectively binds to a chemokine ligand and induces the chemotaxis toward the chemokine ligand.

It is to be understood the chemokine receptor as disclosed herein in some cases refers to not only the naturally occurring chemokine receptors identified in human bodies, but also include chemokine receptors from other sources, such as, but not limited to: (1) naturally occurring chemokine receptors identified in animals, like pigs, dogs, cows, sheep; and (2) non-naturally occurring chemokine receptors, like mutant proteins, chimeric receptors, design proteins with binding affinity to a certain type(s) of chemokines. In some examples, a fragment of a naturally occurring chemokine receptor is also considered a chemokine receptor, if the function of binding and responding to the corresponding chemokine and directing the chemotaxis of the cell is retained in the fragment. As provided herein, in some embodiments, the virus that comprises the exogenous nucleic acid encoding for the chemokine receptor forces a virus-infected cell to express the chemokine receptor as the virus hijacks the host cell's gene expression machinery.

In some embodiments, provided herein is a modified oncolytic virus comprising an exogenous nucleic acid, also referred to herein as a transgene, that encodes for a chemokine receptor. In some cases, the exogenous nucleic acid is a therapeutic transgene. In some cases, the modified oncolytic viruses comprise exogenous nucleic acid that encode for a cytokine receptor whose cognate cytokine is expressed in tumor microenvironments (e.g., IL15-R has a cognate cytokine IL15 expressed in a tumor microenvironment). In some cases, the modified oncolytic viruses encodes for a chemokine receptor(s) whose cognate chemokine(s) are likely to be expressed on tumors (e.g., CXCR4 has a cognate chemokine CXCL12 expressed on a tumor; CCR2 has a target CCL2 expressed on a tumor) and is delivered systemically as a naked virus. Subsequent to entry of the modified oncolytic viruses into the blood stream, by systemic delivery, the viruses infect lymphocytes, such as B-cells, and re-direct the infected B-cells to the tumor, resulting in significantly increased viral load in the tumor. In certain embodiments, the increased viral load in the tumor is achieved soon after the systemic delivery. Ability to deliver the modified oncolytic viruses disclosed herein, in a systemic manner, provides an advantage over traditional intratumoral delivery methods for oncolytic viruses. While intratumoral delivery is helpful in treating easily accessible tumors, in some instances, it is critical to treat inaccessible or metastatic cancer which is allegedly the predominant cause of death from the disease. In this context, it is ineffective to rely on oncolytic viruses delivered intratumorally, as it will need systemic dissemination after administration to the distant sites. However, this dissemination is often transient and ineffective, at least in part, due to the development of immune responses to the viral infection.

Chemokine receptors are divided into different families. Non-limiting examples of chemokine receptors, as described herein include CXC chemokine receptors, CC chemokine receptors, CX3C chemokine receptors and XC chemokine receptors that correspond to the 4 distinct subfamilies of chemokines they bind. Among the CXC chemokine receptors, CXCR1 and CXCR2 are closely related, while CXCR1 binds to CXCL8 and CXCL6, and CXCR2 binds to CXCL1 and CXCL7; CXCR3 binds to CXCL9, CXCL10, and CXCL11; CXCR4 binds to CXCL12 (or SDF-1); CXCR5 binds to CXCL13; CXCR6 binds to CXCL16. Among the CC chemokine receptors, CCRI's ligands include CCL4, CCL5, CCL6, CCL14, CCL15, CCL16, CCL23; CCR2's ligands include CCL2, CCL8, and CCL16; CCR3's ligands include CCL11, CCL26, CCL7, CCL13, CCL15, CCL24, CCL5, CCL28, and CCL18; CCR4's ligands include CCL3, CCL5, CCL17, and CCL22; CCR5's ligands include CCL3, CCL4, CCL5, CCL8, CCL11, CCL13, CCL14, and CCL16; CCR6's ligands include CCL20; CCR7's ligands include CCL19 and CCL21; CCR8's ligands include CCL1, CCL16; CCR9's ligands include CCL25; CCR10's ligand include CCL27, CCL28; CCR11's ligands include CCL19, CCL21, CCL25. CX3C chemokine receptor CX3CR1 has a ligand CXCL1. XC chemokine receptor XCR1 binds to both XCL1 and XCL2.

Non-limiting embodiments of the present disclosure provide a modified oncolytic virus that comprises an exogenous nucleic acid that encodes for a chemokine receptor. In some embodiments, the chemokine receptor is a CXC chemokine receptor, a CC chemokine receptor, a CX3C chemokine receptor, a XC chemokine receptor, or any combinations thereof. In some embodiments, the chemokine receptor is CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, or any combinations thereof.

In certain embodiments, the modified oncolytic virus comprises an exogenous CXCR4-expressing nucleic acid. In certain embodiments, the modified oncolytic virus comprises an exogenous CCR2-expressing nucleic acid. Certain embodiments disclose a modified oncolytic virus comprising an exogenous nucleic acid that encodes for both CXCR4 and CCR2, and both chemokines are expressed from the same virus. Under certain circumstances, CXCL12 and/or CCL2 typically expressed in the tumor microenvironment attracts the CXCR4 and/or CCR2-expressing lymphocytes or other migrating cells that are infected by the modified oncolytic virus, thereby enhancing the tumor-targeted delivery of the modified oncolytic virus. Nucleic acid and amino acid sequences of selected chemokine receptors are listed in Table 7.

TABLE 7
Chemokine receptor sequences.

| SEQ | | |
|---|---|---|
| ID NO: | Description | Amino Acid Sequence |
| 42 | Murine CXCR3 | MYLEVSERQVLDASDFAFLLENSTSPYDYGENESDF |
| | | SDSPPCPQDFSLNFDRTFLPALYSLLFLLGLLGNGAV |
| | | AAVLLSQRTALSSTDTFLLHLAVADVLLVLTLPLW |
| | | AVDAAVQWVFGPGLCKVAGALFNINFYAGAFLLA |
| | | CISFDRYLSIVHATQIYRRDPRVRVALTCIVVWGLC |
| | | LLFALPDFIYLSANYDQRLNATHCQYNFPQVGRTAL |
| | | RVLQLVAGFLLPLLVMAYCYAHILAVLLVSRGQRR |
| | | FRAMRLVVVVVAAFAVCWTPYHLVVLVDILMDVG |
| | | VLARNCGRESHVDVAKSVTSGMGYMHCCLNPLLY |
| | | AFVGVKFREQMWMLFTRLGRSDQRGPQRQPSSSRR |
| | | ESSWSETTEASYLGL |
| 43 | Human CXCR3 isoform 1 | MVLEVSDHQVLNDAEVAALLENFSSSYDYGENESD |
| | | SCCTSPPCPQDFSLNFDRAFLPALYSLLFLLGLLGNG |
| | | AVAAVLLSRRTALSSTDTFLLHLAVADTLLVLTLPL |
| | | WAVDAAVQWVFGSGLCKVAGALFNINFYAGALLL |
| | | ACISFDRYLNIVHATQLYRRGPPARVTLTCLAVWGL |
| | | CLLFALPDFIFLSAHHDERLNATHCQYNFPQVGRTA |
| | | LRVLQLVAGFLLPLLVMAYCYAHILAVLLVSRGQR |
| | | RLRAMRLVVVVVVAFALCWTPYHLVVLVDILMDL |
| | | GALARNCGRESRVDVAKSVTSGLGYMHCCLNPLL |
| | | YAFVGVKFRERMWMLLLRLGCPNQRGLQRQPSSS |
| | | RRDSSWSETSEASYSGL |
| 44 | Human CCR2 var 467 | MLSTSRSRFIRNTNESGEEVTTFFDYDYGAPCHKFD |
| | | VKQIGAQLLPPLYSLVFIFGFVGNMLVVLILINCKKL |
| | | KCLTDIYLLNLAISDLLFLITLPLWAHSAANEWVFG |
| | | NAMCKLFTGLYHIGYFGGIFFIILLTIDRYLAIVHAVF |
| | | ALKARTVTFGVVTSVITWLVAVFASVPGIIFTKCQK |
| | | EDSVYVCGPYFPRGWNNFHTIMRNILGLVLPLLIMV |
| | | ICYSGILKTLLRCRNEKKRHRAVRVIFTIMIVYFLFW |
| | | TPYNIVILLNTFQEFFGLSNCESTSQLDQATQVTETL |
| | | GMTHCCINPIIYAFVGEKFRSLFHIALGCRIAPLQKP |
| | | VCGGPGVRPGKNVKVTTQGLLDGRGKGKSIGRAPE |
| | | ASLQDKEGA |
| 45 | CCR2 var 502 | MLSTSRSRFIRNTNESGEEVTTFFDYDYGAPCHKFD |
| | | VKQIGAQLLPPLYSLVFIFGFVGNMLVVLILINCKKL |
| | | KCLTDIYLLNLAISDLLFLITLPLWAHSAANEWVFG |
| | | NAMCKLFTGLYHIGYFGGIFFIILLTIDRYLAIVHAVF |
| | | ALKARTVTFGVVTSVITWLVAVFASVPGIIFTKCQK |
| | | EDSVYVCGPYFPRGWNNFHTIMRNILGLVLPLLIMV |
| | | ICYSGILKTLLRCRNEKKRHRAVRVIFTIMIVYFLFW |
| | | TPYNIVILLNTFQEFFGLSNCESTSQLDQATQVTETL |
| | | GMTHCCINPIIY AFVGEKFRSLFHIALGCRIAPLQKP |
| | | VCGGPGVRPGKNVKVTTQGLLDGRGKGKSIGRAPE |
| | | ASLQDKEGA |
| 46 | Human CXCR2 | MEDFNMESDSFEDFWKGEDLSNYSYSSTLPPFLLD |
| | | AAPCEPESLEINKYFWIIYALVFLLSLLGNSLVMLVI |
| | | LYSRVGRSVTDVYLLNLALADLLFALTLPIWAASK |
| | | VNGWIFGTFLCKWSLLKEVNFYSGILLLACISVDRY |
| | | LAIVHATRTLTQKRYLVKFICLSIWGLSLLLALPVL |
| | | LFRRTVYSSNVSPACYEDMGNNTANWRMLLRILP |
| | | QSFGFIVPLLIMLFCYGFTLRTLFKAHMGQKHRAM |
| | | RVIFAWLIFLLCWLPYNLVLLADTLMRTQVIQETC |
| | | ERRNHIDRALDATEILGILHSCLNPLIYAFIGQKFRH |
| | | GLLKILAIHGLISKDSLPKDSRPSFVGSSSGHTSTTL |
| 47 | Human CCR2 | MLSTSRSRFIRNTNESGEEVTTFFDYDYGAPCHKFD |
| | | VKQIGAQLLPPLYSLVFIFGFVGNMLVVLILINCKK |
| | | LKCLTDIYLLNLAISDLLFLITLPLWAHSAANEWVF |
| | | GNAMCKLFTGLYHIGYFGGIFFIILLTIDRYLAIVHA |
| | | VFALKARTVTFGVVTSVITWLVAVFASVPGIIFTKC |
| | | QKEDSVYVCGPYFPRGWNNFHTIMRNILGLVLPLL |
| | | IMVICYSGILKTLLRCRNEKKRHRAVRVIFTIMIVYF |
| | | LFWTPYNIVILLNTFQEFFGLSNCESTSQLDQATQV |
| | | TETLGMTHCCINPIIYAFVGEKFRSLFHIALGCRIAP |
| | | LQKPVCGGPGVRPGKNVKVTTQGLLDGRGKGKSI |
| | | GRAPEASLQDKEGA |
| 48 | Human CXCR4, Y157A | MEGISIYTSDNYTEEMGSGDYDSMKEPCFREENAN |
| | | FNKIFLPTIYSIIFLTGIVGNGLVILVMGYQKKLRSM |
| | | TDKYRLHLSVADLLFVITLPFWAVDAVANWYFGN |
| | | FLCKAVHVIYTVNLYSSVLILAFISLDRYLAIVHAT |
| | | NSQRPRKLLAEKVVAVGVWIPALLLTIPDFIFANVS |
| | | EADDRYICDRFYPNDLWVVVFQFQHIMVGLILPGI |
| | | VILSCYCIIISKLSHSKGHQKRKALKTTVILILAFFAC |
| | | WLPYYIGISIDSFILLEIIKQGCEFENTVHKWISITEA |
| | | LAFFHCCLNPILYAFLGAKFKTSAQHALTSVSRGSS |
| | | LKILSKGKRGGHSSVSTESESSSFHSS |
| 49 | Mouse CXCR4, Y159A | MEPISVSIYTSDNYSEEVGSGDYDSNKEPCFRDENV |
| | | HFNRIFLPTIYFIIFLTGIVGNGLVILVMGYQKKLRS |
| | | MTDKYRLHLSVADLLFVITLPFWAVDAMADWYF |
| | | GKFLCKAVHIIYTVNLYSSVLILAFISLDRYLAIVHA |
| | | TNSQRPRKLLAEKAVAVGVWIPALLLTIPDFIFADV |
| | | SQGDISQGDDRYICDRLYPDSLWMVVFQFQHIMV |
| | | GLVLPGIVILSCYCIIISKLSHSKGHQKRKALKTTVIL |
| | | ILAFFACWLPYYVGISIDSFILLGVIKQGCDFESIVH |
| | | KWISITEALAFFHCCLNPILYAFLGAKFKSSAQHAL |
| | | NSMSRGSSLKILSKGKRGGHSSVSTESESSSFHSS |
| 50 | Human CXCR4, wildtype | MEGISIYTSDNYTEEMGSGDYDSMKEPCFREENAN |
| | | FNKIFLPTIYSIIFLTGIVGNGLVILVMGYQKKLRSM |
| | | TDKYRLHLSVADLLFVITLPFWAVDAVANWYFGN |
| | | FLCKAVHVIYTVNLYSSVLILAFISLDRYLAIVHAT |
| | | NSQRPRKLLAEKVVYVGVWIPALLLTIPDFIFANVS |
| | | EADDRYICDRFYPNDLWVVVFQFQHIMVGLILPGI |
| | | VILSCYCIIISKLSHSKGHQKRKALKTTVILILAFFAC |
| | | WLPYYIGISIDSFILLEIIKQGCEFENTVHKWISITEA |
| | | LAFFHCCLNPILYAFLGAKFKTSAQHALTSVSRGSS |
| | | LKILSKGKRGGHSSVSTESESSSFHSS |
| 51 | Human CXCR4, Y157A var | MEGISIYTSDNYTEEMGSGDYDSMKEPCFREENAN |
| | | FNKIFLPTIYSIIFLTGIVGNGLVILVMGYQKKLRSM |
| | | TDKYRLHLSVADLLFVITLPFWAVDAVANWYFGN |
| | | FLCKAVHVIYTVNLYSSVLILAFISLDRYLAIVHAT |
| | | NSQRPRKLLAEKVVAVGVWIPALLLTIPDFIFANVS |
| | | EADDRYICDRFYPNDLWVVVFQFQHIMVGLILPGI |
| | | VILSCYCIIISKLSHSKGHQKRKALKTTVILILAFFAC |
| | | WLPYYIGISIDSFILLEIIKQGCEFENTVHKWISITEA |
| | | LAFFHCCLNPILYAFLGAKFKTSAQHALTSVSRGSS |
| | | LKILSKGKRGGHSSVSTESESSSFHSS |
| 52 | Mouse CXCR4, Y159A var. | MEPISVSIYTSDNYSEEVGSGDYDSNKEPCFRDENV |
| | | HFNRIFLPTIYFIIFLTGIVGNGLVILVMGYQKKLRS |
| | | MTDKYRLHLSVADLLFVITLPFWAVDAMADWYF |
| | | GKFLCKAVHIIYTVNLYSSVLILAFISLDRYLAIVHA |
| | | TNSQRPRKLLAEKAVAVGVWIPALLLTIPDFIFADV |
| | | SQGDISQGDDRYICDRLYPDSLWMVVFQFQHIMV |
| | | GLVLPGIVILSCYCIIISKLSHSKGHQKRKALKTTVIL |
| | | ILAFFACWLPYYVGISIDSFILLGVIKQGCDFESIVH |
| | | KWISITEALAFFHCCLNPILYAFLGAKFKSSAQHAL |
| | | NSMSRGSSLKILSKGKRGGHSSVSTESESSSFHSS |
| 53 | Human wild-type CXCR4 | MEGISIYTSDNYTEEMGSGDYDSMKEPCFREENAN |
| | | FNKIFLPTIYSIIFLTGIVGNGLVILVMGYQKKLRSM |
| | | TDKYRLHLSVADLLFVITLPFWAVDAVANWYFGN |
| | | FLCKAVHVIYTVNLYSSVLILAFISLDRYLAIVHAT |
| | | NSQRPRKLLAEKVVYVGVWIPALLLTIPDFIFANVS |
| | | EADDRYICDRFYPNDLWVVVFQFQHIMVGLILPGI |
| | | VILSCYCIIISKLSHSKGHQKRKALKTTVILILAFFAC |
| | | WLPYYIGISIDSFILLEIIKQGCEFENTVHKWISITEA |
| | | LAFFHCCLNPILYAFLGAKFKTSAQHALTSVSRGSS |
| | | LKILSKGKRGGHSSVSTESESSSFHSS |
| 54 | Human CCL5 | MKVSAAALAVILIATALCAPASASPYSSDTTPCCFA |
| | | YIARPLPRAHIKEYFYTSGKCSNPAWFVTRKNRQV |
| | | CANPEKKWVREYINSLEMS |
| 55 | Human CCR5 | MDYQVSSPIYDINYYTSEPCQKINVKQIAARLLPPL |
| | | YSLVFIFGFVGNMLVILILINCKRLKSMTDIYLLNLA |
| | | ISDLFFLLTVPFWAHYAAAQWDFGNTMCQLLTGL |
| | | YFIGFFSGIFFIILLTIDRYLAWHAVFALKARTVTFG |
| | | WTSVITWWAVFASLPGIIFTRSQKEGLHYTCSSHFP |
| | | YSQYQFWKNFQTLKIVILGLVLPLLVMVICYSGILK |
| | | TLLRCRNEKKRHRAVRLIFTIMIVYFLFWAPYNIVL |
| | | LLNTFQEFFGLNNCSSSNRLDQAMQVTETLGMTHC |
| | | CINPIIYAFVGEKFRNYLLVFFQKHIAKRFCKCCSIF |
| | | QQEAPERASSVYTRSTGEQEISVGL |

In compositions provided herein, an oncolytic virus gene may be mutated or replaced with nucleic acid encoding for a chemokine receptor as listed in Table 7. In some embodiments, the chemokine receptor is a murine CXCR3 described by SEQ ID NO: 42. In some embodiments, the encoded murine CXCR3 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 42. In some embodiments, the chemokine receptor is a human CXCR3 described by SEQ ID NO: 43. In some embodiments, the encoded human CXCR3 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 43.

In some embodiments, provided herein is a modified oncolytic virus comprising an exogenous nucleic acid that encodes for a membrane associated protein that degrades hyaluronan, such as a hyaluronidase. In some embodiments, provided herein is a modified oncolytic virus comprising an exogenous nucleic acid, also referred to herein as a transgene, that encodes for a chemokine receptor. In some cases, the exogenous nucleic acid is a therapeutic transgene.

### Promoters

Provided herein are compositions comprising nucleic acids, wherein the nucleic acid encodes for at least one promoter region. A promoter region, or promoter, or promoter element, or regulatory region, refers to a nucleic acid sequence to which proteins bind to initiate transcription. Promoters are typically located 5′, or upstream, to a DNA coding region which they control. In some embodiments, a nucleic acid described herein comprises one promoter. In some embodiments, the one promoter drives transcription of all polypeptides encoded on the nucleic acid. In some embodiments, a nucleic acid described herein comprises a separate promoter for each polypeptide encoded on the nucleic acid. In some embodiments, the nucleic acid comprises two promoters, each driving transcription of one of two polypeptides encoded on the nucleic acid.

Timing of expression can be modulated by the structure of the promotor regulating the gene expression. The number and affinity of transcription factor binding sites determines the relative timing of expression between different promoter regions. A promoter with more transcription factor binding sites and/or higher binding affinity can drive expression earlier than a promoter with fewer or lower affinity binding sites.

Application of the relative temporal expression of proteins, can be leveraged to express particular factors from modified viruses as described herein either earlier or later in the infection process. An early promoter has repeated transcription factor binding sites. A late promoter has fewer binding sites than an early promoter. In some embodiments, a receptor is expressed using an early promoter. Expression early in infection allows for expression and processing by the cell, before cellular processes are disrupted. In some embodiments, one or more cytokines are expressed using a late promoter.

Provided herein, in some embodiments, are promoters comprising P7.5, P28, P135, TK promoter, A52R promoter, 454 promoter, PB8, LEO, PF11, F7L, H5R, mH5, H1L, A1L, J3R, E4L, I1L, I3L, I4L, I5L, I7L, T7, I2L, FP4b, ATI, P11, PFL1, L4R, T7 promoter, 28 kDa promoter, a short synthetic promoter (SSP), or any functional variant or combination thereof. In some embodiments, the promoter comprises an early promoter. In some embodiments, the early promoter comprises A52R, PB8, mH5, I4L, LEO, PF11, I3L, P7.5, TK promoter, F7L, H5R, a short synthetic promoter (SSP) or any variation or combination thereof. In some embodiments, the promoter comprises a late promoter. In some embodiments, the late promoter comprises SSP, P7.5, P28, P135, TK promoter, F7L, H5R, H1L, A1L, J3R, E4L, I1L, I5L, I7L, T7, I2L, FP4b, ATI, P11, PFL1, L4R, 28 kDa promoter or any functional variant or combination thereof. Sequences of selected promoters are listed in Table 8.

TABLE 8
Promoter nucleic acid sequences

| SEQ ID | | |
|---|---|---|
| NO: | Description | Nucleic Acid Sequence |
| 56 | P135 | GCGTCCTAAAATTAAAACAAATGATATAA |
| | (VACWR135) | |
| 57 | P7.5 | TCACTAATTCCAAACCCACCCGCTTTTTATAGTAAGTTTTTCAC |
| | (vaccinia) | CCATAAATAATAAATACAATAATTAATTTCTCGTAAAAGTAGA |
| | | AAATATATTCTAATTTATTGCACGGTAAGGAAGTAGATCATAA |
| 58 | P28 | AAGCTCACAAAAAAAACTTCTCTAAATAGATTCGAGC |
| 59 | Strong | GTGATGCGGTTTTGGCAGTACATCAATGGGCGTGGATAGCGGT |
| | vaccinia virus | TTGACTCACGGGGATTTCCAAGTCTCCACCCCATTGACGTCAA |
| | early promoter | TGGGAGTTTGTTTTGGCACCAAAATCAACGGGACTTTCCAAAA |
| | | TGTCGTAACAACTCCGCCCCATTGACGCAAATGGGCGGTAGG |
| | | CGTGTACGGTGGGAGGTCTATATAAGCAGAGCT |
| 60 | I3L | AGATAAAGTGAAAATATATATCATTATATTACAAAGTACAATT |
| | | ATTTAGGTTTAATCAGA |
| 61 | I4L | AATCCCTATTAATGAAAAGTTAAATAATTTTTTTATTACACCA |
| | | ACAAAAAMT |
| 62 | 454 promoter | GGATCATAAAAATATAGTAGAATTTCATTTTGTTTTTTTCTATG |
| | | CTATAAATAGCTTCTCGAGC |
| 63 | Human PGK | GGGGTTGGGGTTGCGCCTTTTCCAAGGCAGCCCTGGGTTTGCG |
| | promoter | CAGGGACGCGGCTGCTCTGGGCGTGGTTCCGGGAAACGCAGC |
| | | GGCGCCGACCCTGGGTCTCGCACATTCTTCACGTCCGTTCGCA |
| | | GCGTCACCCGGATCTTCGCCGCTACCCTTGTGGGCCCCCCGGC |
| | | GACGCTTCCTGCTCCGCCCCTAAGTCGGGAAGGTTCCTTGCGG |
| | | TTCGCGGCGTGCCGGACGTGACAAACGGAAGCCGCACGTCTC |
| | | ACTAGTACCCTCGCAGACGGACAGCGCCAGGGAGCAATGGCA |
| | | GCGCGCCGACCGCGATGGGCTGTGGCCAATAGCGGCTGCTCA |
| | | GCAGGGCGCGCCGAGAGCAGCGGCCGGGAAGGGGCGGTGCG |
| | | GGAGGCGGGGTGTGGGGCGGTAGTGTGGGCCCTGTTCCTGCC |
| | | CGCGCGGTGTTCCGCATTCTGCAAGCCTCCGGAGCGCACGTCG |
| | | GCAGTCGGCTCCCTCGTTGACCGAATCACCGACCTCTCTCCCC |
| | | AG |

Compositions provided herein may comprise a P7.5 promoter and a P28 promoter. In some embodiments, the P7.5 promoter drives expression of a region encoding for an IL-12 polypeptide. In some embodiments, the P28 promoter drives transcription of a TGFbf polypeptide. A schematic representation of promoter and gene expression construct is shown in FIG. 2. In some embodiments, the P7.5 promoter comprises a nucleic acid sequence as set forth in SEQ ID NO: 57. In some embodiments, the P28 promoter comprises a nucleic acid sequence as set forth in SEQ ID NO: 58.

Compositions provided herein may comprise a P135 promoter and a P7.5 promoter. In some embodiments, the P135 promoter drives expression of a region encoding for an IL-12 polypeptide. In some embodiments, the P7.5 promoter drives expression of a region encoding for a TGF-beta polypeptide. In some embodiments, the P7.5 promoter comprises a nucleic acid sequence as set forth in SEQ ID NO: 57. In some embodiments, the P135 promoter comprises a sequence as set forth in SEQ ID NO: 56.

Compositions provided herein may comprise a P7.5 promoter (SEQ ID NO: 57), a P135 promoter (SEQ ID NO: 56), and an A52R promoter. In some embodiments, the P135 promoter drives expression of a region encoding for an IL-12 polypeptide. In some embodiments, the P7.5 promoter drives expression of a region encoding for a TGF-beta polypeptide. In some embodiments, the A52R promoter drives expression of a region encoding for a CXCR3 receptor. In some embodiments, expression of the CXCR3 receptor occurs prior to expression of the IL-12 polypeptide or the TGF-beta polypeptide.

Provided herein are compositions comprising an expression construct comprising, in 5′ to 3′ order, an IL-12 beta subunit, a linker, an IL-12 alpha subunit, an IL-12 signal sequence, and a TGF-beta variant. In some embodiments, the expression construct comprises, in 5′ to 3′ order, hIL-12b (SEQ ID NO: 17), a flexible linker (SEQ ID NO: 18), hIL-12a (SEQ ID NO: 16), a human IgEsig sequence (SEQ ID NO: 37), and a TGF-beta 2 variant sequence (TGFbv2) (SEQ ID NO: 8). In some embodiments, the expression construct comprises, in 5′ to 3′ order, mIL-12b (SEQ ID NO: 14), a flexible linker (SEQ ID NO: 18), mIL-12a (SEQ ID NO: 16), murine IL-2sig (SEQ ID NO: 36), and a TGF-beta 2 variant sequence (TGFbv1) (SEQ ID NO: 7). In some embodiments, the expression construct comprises, in 5′ to 3′ order, mIL-12 (SEQ ID NO: 12), and TGFbf1 (SEQ ID NO: 40). In some embodiments, the expression construct comprises, in 5′ to 3′ order, hIL-12 (SEQ ID NO: 15), and TGFbf2 (SEQ ID NO: 41). In some embodiments, exogenous nucleic acids described herein are incorporated into a viral genome.

Provided herein are compositions comprising an expression construct comprising, in 5′ to 3′ order, an IL-12 signal sequence, a TGF-beta variant an IL-12 beta subunit, a linker, and an IL-12 alpha subunit. In some embodiments, the expression construct comprises, in 5′ to 3′ order, a human IgE sig sequence (SEQ ID NO: 37), a TGF-beta 2 variant sequence (TGFbv2) (SEQ ID NO: 8), hIL-12b (SEQ ID NO: 17), a flexible linker (SEQ ID NO: 18), and hIL-12a (SEQ ID NO: 16). In some embodiments, the expression construct comprises, in 5′ to 3′ order, murine IL-2sig (SEQ ID NO: 36), a TGF-beta 2 variant sequence (TGFbv1) (SEQ ID NO: 7), mIL-12b (SEQ ID NO: 14), a flexible linker (SEQ ID NO: 18), and mIL-12a (SEQ ID NO: 16). In some embodiments, the expression construct comprises, in 5′ to 3′ order, TGFbf1 (SEQ ID NO: 40), and mIL-12 (SEQ ID NO: 12). In some embodiments, the expression construct comprises, in 5′ to 3′ order, TGFbf2 (SEQ ID NO: 41), and hIL-12 (SEQ ID NO: 15).

### Vectors

Vectors can be used to deliver exogenous nucleic acids to cells for replication or expression. In some embodiments, a vector is a particle, comprising a plasmid, a viral vector, a cosmid, or an artificial chromosome. A vector generally carries the exogenous nucleic acid inserted in a “backbone” nucleic acid of the vector. In some embodiments, the exogenous nucleic acid is a DNA or an RNA. In some embodiments, a vector comprises at least one promoter sequence that drives expression of the exogenous nucleic acid.

### Oncolytic Viruses

Oncolytic viruses can preferentially infect and kill cancer cells. Oncolysis of infected cancer cells can perpetuate the spread of the virus to surrounding tissue and can also stimulates a host anti-tumor immune response. Provided herein are compositions comprising an oncolytic virus wherein the oncolytic virus comprises a modified nucleic acid described herein. Oncolytic viruses, as used herein, kill cancer or tumor cells through mechanisms such as the direct lysis of said cells, by stimulating immune response towards said cells, apoptosis, expression of toxic proteins, autophagy and shutdown of protein synthesis, induction of anti-tumoral immunity, or any combinations thereof. In some embodiments, an oncolytic virus as described herein replicates within a cell. In some embodiments, an oncolytic virus as described herein replicates within a tumor cell, an immune cell, a somatic cell, a hemopoietic cell, or another type of cell. Exemplary oncolytic viruses for inclusion in a composition described herein include, without limitation, a poxvirus, a vaccinia virus, an adeno associated virus, an adenovirus, a reovirus, a lentivirus, a herpes simplex virus, a vesicular stomatitis virus, a mengovirus, a myxoma virus, Newcastle disease virus, measles virus, or polio virus. These oncolytic viruses have a proclivity to specifically target cancer cells, and upon virus replication cause significant cell death and tumor regression. In some embodiments, the oncolytic virus is a vaccinia virus. Exemplary vaccinia viruses include, without limitation, the following strains for modification by inclusion of a construct described herein: Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Furthermore, in some embodiments, the recombinant vaccinia viruses are modified versions of a wild type or attenuated vaccinia virus strain. Nonlimiting examples of vaccinia virus strains include a Western Reserve strain of vaccinia virus, a Copenhagen strain, a IHD strain, a Wyeth (NYCBOH) strain, a Tian Tan strain, a Lister strain, a USSR strain, an Ankara strain, an NYVAC strain, an Ankara (MVA) strain, a Paris strain, a Bern strain, a Temple of Heaven strain, a Dairen strain, an EM-63 strain, an Evans strain, a King strain, a Patwadangar strain, or a Tash Kent strain. The base vaccinia virus strain modified as set forth herein optionally comprises one or more mutation(s) relative to its parent strain, for example, but not limited to, one or more of the following: deletion in TK (also referred to herein as “TK-”) and deletion in A52R (also referred to herein as “A52R-”). Vaccinia viruses are optionally recombinant or selected to have low toxicity and to accumulate in the target tissue. In some embodiments, the modifications in the viral backbone/viral genome are modifications that render the vaccinia virus non-replicating or comprise a poor replicative capacity. Non-limiting examples of such modifications include mutations in the following viral genes: A1, A2, VH1, A33, and I7. In some embodiments, the viral backbone mutation is selected from the group consisting of: a complete or partial deletion of the A52R gene; a complete or partial deletion of the TK gene; a complete or partial deletion of the B15R gene; a complete or partial deletion of the K7R gene; a complete or partial deletion of the B14R gene; a complete or partial deletion of the N1L gene; a complete or partial deletion of the K1L gene; a complete or partial deletion of the M2L gene; a complete or partial deletion of the A49R gene; a complete or partial deletion of the VH1 gene; a complete or partial deletion of A33 gene; a complete or partial deletion of A1; a complete or partial deletion of A2 gene; a complete or partial deletion of 17 gene, and a complete or partial deletion of the A46R gene. As used herein, the reference to a viral gene is made by reference to the protein encoded by the gene (e.g., A33 gene means a gene that encodes for the A33 protein). In some embodiments, the viral backbone mutation, including any combinations of substitution, insertion, and deletion, result in a sequence with less than 100%, 99%, 98%, 97%, 96%, 95%, 94%, 93%, 92%, 91%, 90% or less sequence homology to the wild-type sequence of the viral gene or a viral protein encoded by the gene. The viral gene and protein encoded by the same, in some embodiments, is selected from the group consisting of: B15R, K7R, B14R, N1L, K1L, M2L, A49R, VH1, A33, A1, A2, 17, and A46R. In some embodiments, the viral backbone comprises 1, 2, 3, 4, 5, or more mutations in the amino acid sequence of the viral protein (e.g., a viral antigen). The viral antigen is in some examples selected from the group consisting of: B15R, K7R, B14R, N1L, K1L, M2L, A49R, VH1, A33, A1, A2, 17, and A46R. The disclosure provides in some embodiments, recombinant vaccinia viruses containing one more mutation(s) in the genome of the virus (virus back bone) such that the mutation increases the T-cell arm of the immune response. A mutation may be addition, deletion, or substitution of one or more nucleic acid(s) in the viral genome (wild type or attenuated native strains of vaccinia virus). In non-limiting examples, the mutation is complete or partial deletion of genes that are known to inhibit cytokines involved in the Th1 immune response. As non-limiting examples, the mutation is a deletion of nucleic acid encoding for B8R (interferon gamma (IFN-g) binding proteins); C12L (interleukin-18 (IL-18) binding proteins). In a further non-limiting example, the mutation is a complete or partial deletion of genes in innate immune signaling. As non-limiting examples, the mutation is a deletion of nucleic acid encoding for (B18R (type I interferon (IFN)-binding proteins); A52R (nuclear factor KB (NF-κB) inhibitor proteins); E3L (protein kinase (PKR) inhibitors); C4, C16 (STING pathway inhibitors.

An oncolytic virus, as described herein, contains one or more additional insertions or partial insertions of exogenous nucleic acids that encode for one or more proteins. In some embodiments, the one or more proteins include a chemokine receptor or a functional variant thereof, TGF-beta inhibitor or a functional variant thereof, or interleukin-12 or a functional variant thereof. In some embodiments, the one or more proteins include a TGF-beta inhibitor or a functional variant thereof and interleukin-12 or a functional variant thereof. Exemplary chemokine receptors for inclusion include, without limitation, wild type and/or mutant type CXCR3, CXCR4, CCR2, or CCL2. A vaccinia virus of the current disclosure further contains one or more additional deletions or partial deletions of one or more genes from A52R, B15R, K7R, A46R, N1L, E3L, K1L, M2L, C16, N2R, B8R, B18R, VH1 and a functional domain or fragment or variant thereof, or any combinations thereof. In some cases, the vaccinia virus provided herein contains a complete or partial deletion of at least one of: A52R or TK viral genes, and insertion of an exogenous nucleic acid encoding for one or more proteins (e.g., one or more immune modulator proteins).

In some embodiments, the oncolytic virus is a modified oncolytic virus that has one or more modifications that results in a greater therapeutic effect against tumor cells, as compared to an otherwise identical virus that does not comprise the modifications. In some non-limiting examples, the greater therapeutic effect includes each or any combinations of: enhanced immune evasion of the virus, enhanced tumor-targeted systemic delivery of the virus, enhanced intratumoral and intertumoral spreading of the virus, and enhanced tumor-specific replication of the virus, or release of immune modulators and anti-tumor agents into the extracellular matrix. The modified oncolytic virus of this disclosure, in some instances, is utilized as a platform vector for systemic delivery.

Oncolytic viruses as described herein comprise exogenous nucleic acids described herein. In some embodiments, the oncolytic virus provided herein comprises a complete or partial deletion of the TK gene and an insertion of a region encoding for at least one of a Transforming growth factor-beta inhibitor and a cytokine, such as IL-12. Exemplary sequences for incorporation are described previously herein.

In some embodiments, the oncolytic virus provided herein contains a complete or partial deletion of the A52R gene and an insertion of a region encoding for a chemokine receptor. In some embodiments, the chemokine receptor comprises CXCR3. In some embodiments, region encoding for a chemokine receptor comprises a sequence selected from Table 7. In some embodiments, the promoter driving expression of the chemokine receptor is an early promoter, a late promoter, a strong early promoter, a weak early promoter, a strong late promoter, a weak late promoter, or any combination thereof. In some embodiments, an A52R promoter drives expression of the chemokine receptor. In some embodiments, an A52R promoter drives expression of the region encoding for CXCR3.

In some embodiments of the present disclosure, provided is a modified oncolytic virus comprising a modification that enhances tumor-targeted systemic delivery of the virus. Typically, oncolytic viruses are either be (a) administered systemically (b) inoculated topically over the tumor or, (c) injected directly into the tumor (“intratumoral delivery”). In some embodiments, systemic delivery of the oncolytic virus affords the opportunity to treat both the primary tumor and any overt or undiagnosed metastatic deposits simultaneously. As a result, this method of delivery is a very attractive option for the treatment of patients with advanced/metastatic disease or patients with inaccessible disease such as those with pancreatic cancer or brain cancer, where access is difficult for example due to physiological barriers, such as blood-brain barrier. However, barriers exist for successful systemic delivery of many oncolytic viruses. For instance, in some cases, as described above, host defense limits most oncolytic viruses' ability to infect tumors after systemic administration. Blood cells, complement, antibodies, and antiviral cytokines, as well as nonspecific uptake by other tissues such as the lung, liver and spleen, tissue-resident macrophages, and additionally poor virus escape from the vascular compartment are among the main barriers to systemic delivery of oncolytic viruses. In some embodiments of the present disclosure, disclosed oncolytic viruses comprise a modification that promotes the persistent existence of the virus in the circulation system, at least through, as abovementioned, enhancement of immune evasion. Alternatively, enhanced tumor-targeted delivery of the virus is desirable under certain circumstances, as it not only increases therapeutic efficacy against cancer, but also alleviates the safety concerns around virus-mediated oncotherapy as the non-tumor infection is limited, avoiding the undesired side effects of viral infection. Certain embodiments herein relate to an oncolytic virus comprising a modification that promotes the tumor-targeted delivery of the virus.

In some embodiments of the present disclosure, provided is a modified oncolytic virus comprising a modification that enhances intratumoral and intertumoral spreading of the virus. Enhanced spreading of the oncolytic virus within and between tumors is an effective manner to boost the therapeutic efficacy by increasing the number of the cancer cells that are infected by the virus. Provided herein, in some embodiments, is a modified oncolytic virus that comprises an exogenous nucleic acid. Provided herein, in some embodiments, is a modified oncolytic virus that comprises a modification in the genome of the virus. Provided herein, in some embodiments, is a modified oncolytic virus that comprises an exogenous nucleic acid as well as a modification in the genome of the virus.

In some embodiments, the oncolytic viruses include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use. In some embodiments, the oncolytic virus is a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a retrovirus, a mengovirus, or a myxoma virus. In certain embodiments, the oncolytic virus is a poxvirus. In certain embodiments, the oncolytic virus is a vaccinia virus.

In some embodiments, a modified oncolytic virus is employed. In general, such a virus comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of an exogenous protein or modified viral protein to the viral capsid.

In some embodiments, the modified oncolytic virus comprises a mutation or deletion of the TK gene and further comprises an exogenous nucleic acid that encodes for a TGF-beta inhibitor. In some embodiments, the modified oncolytic virus comprises a mutation or deletion of the TK gene, and further comprises an exogenous nucleic acid that encodes for a TGF-beta inhibitor, and an exogenous nucleic acid that encodes for a cytokine, e.g., IL-12. In some embodiments, an exogenous nucleic acid encoding for IL-12 is inserted 5′ to an exogenous nucleic acid encoding for a TGF-beta inhibitor. In some embodiments, an exogenous nucleic acid encoding for a TGF-beta inhibitor is inserted 5′ to an exogenous nucleic acid encoding for IL-12.

In some embodiments, the modified oncolytic virus comprises a mutation or deletion of the A52R gene and further comprises an exogenous nucleic acid that encodes for a CXCR3 receptor. In some embodiments, the modified oncolytic virus comprises a mutation or deletion of the A52R gene wherein the A52R promoter is maintained, and further comprises an exogenous nucleic acid that encodes for a CXCR3 receptor.

In some embodiments, in the modified oncolytic virus, such as in an oncolytic vaccinia virus, the viral TK gene is be replaced with a TK gene from a herpes simplex virus (HSV-TK). The HSV TK optionally functions as a substitute for the deleted TK and has multifaceted advantages. For instance, in some embodiments, HSV TK is used as an additional therapeutic prodrug converting enzyme for converting ganciclovir (GCV) into its cytotoxic metabolite in a tumor. In addition to the added therapeutic effect, this modification also serves as a suicide gene, e.g., vaccinia expressing cells are killed efficiently through addition of GCV, thereby shutting down the virus in the case of an adverse event or uncontrolled replication. Thus, in some instances, the modified oncolytic virus of this disclosure acts as a safety switch. In additional examples, a mutated version of the HSV TK is used to allow for PET imaging of labelled substrates with greatly increased sensitivity. Thus, in some cases, the modified oncolytic virus, comprising an HSV TK that is used in PET imaging, can act as a reporter of viral replication in vivo to determine therapeutic activity early after treatment.

In some cases, the modified oncolytic virus comprises a full-length viral backbone gene or viral backbone protein described above, or truncated versions thereof, or functional domains thereof, or fragments thereof, or variants thereof. In various examples, the modified oncolytic virus comprises a mutation or deletion of one or more of viral backbone genes or viral backbone proteins, as described above. Mutations of the viral backbone genes and viral backbone proteins comprise insertion, deletion, substitution, or modifications of nucleic acids in nucleic acid sequences and amino acids in protein sequences. Deletion comprises, in some examples, a complete or partial deletion of the viral backbone gene or protein.

In some embodiments, the modification of the oncolytic virus results in at least about 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 7.5×10⁵, 10⁶, 2.5×10⁶, 5×10⁶, 7.5×10⁶, 10⁷, 2.5×10⁷, 5×10⁷, 7.5×10⁷, 10⁸, 2.5×10⁸, 5×10⁸, 7.5×10⁸, 10⁹, 2.5×10⁹, 5×10⁹, 7.5×10⁹, 10¹⁰ or even more folds increase in the efficacy of tumor-targeted systemic delivery of the virus, as compared to an otherwise identical oncolytic virus that does not comprise the modification. In certain embodiments, the efficacy of tumor-targeted systemic delivery of the virus is measured by quantifying the viruses infecting the tumor cells, and optionally, in contrast with the viruses infecting non-tumor cells in the body. For instance, in some cases, the quantification of the virus is performed by staining the viral particles in tissue sections, or blood smear in the cases of leukemia, lymphoma, or myeloma. In some cases, such quantification is performed by reporter molecule(s) that is/are engineered to be expressed by the viruses, e.g., luciferase, and fluorescent proteins. In some cases, such quantification is performed by quantifying the viral genome in the tumor. Without being limited, it is also possible to measure the tumor-targeted systemic delivery of the virus by quantifying certain downstream effect(s) of viral infection in tumor cells, like cytokines in response to viral infection or lymphocyte accumulation. In some embodiments, the oncolytic virus comprises an exogenous nucleic acid that encodes for CXCR3, CXCR4, CCR2, or any combination thereof. In some embodiments, the presence of the exogenous nucleic acid results in about 5 to 10 folds increase in the efficacy of tumor-target systemic delivery of the virus, as compared to an otherwise identical oncolytic virus that does not comprise the exogenous nucleic acid.

In some embodiments, provided herein is a modified oncolytic virus that comprises an exogenous nucleic acid that encodes for a chemokine receptor, and the forced expression of chemokine receptor by the modified oncolytic virus results in boosted immune responses against the infected tumor. Following infecting the tumor, the modified oncolytic viruses replicate in the tumor cells and result in the expression of the chemokine receptors on the surface of the tumor cells. These membrane receptors function as decoy receptors, binding and sequestering the immunosuppressive chemokines within the tumor (e.g., CXCL12 and/or CCL2). Consequently, the immunosuppressive microenvironment in the tumor is altered, leading to enhanced immunotherapeutic activity of the modified oncolytic virus, as compared to an otherwise identical virus that does not comprise the nucleic acid encoding for the chemokine receptor. In some embodiments, the increase in immunotherapeutic activity is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5 ×10⁵, 5×10⁵, 10⁶ or even higher folds. Without being limited, the increased immunotherapeutic activity is reflected by increased B cell accumulation in the tumor, increased T cell response to tumor-related immunogens, or both. B cell accumulation is measured, for example, by quantifying the B cells in the tumor, and T cell immuno-activity is measured by, for example, interferon-Y (interferon-gamma) secretion in ELISPOT assays.

In some embodiments, provided herein is a modified oncolytic virus that comprises an exogenous nucleic acid that encodes for a chemokine receptor, and the forced expression of chemokine receptor by the modified oncolytic virus results in increased replication of the virus in tumor cells, as compared to an otherwise identical virus that does not comprise the nucleic acid encoding for the chemokine receptor. In some embodiments, the modified oncolytic virus comprises an exogenous CXCR3-expressing nucleic acid. In some embodiments, the modified oncolytic virus comprises an exogenous CCR2-expressing nucleic acid, which increases the tumor-specific replication of the virus. In some embodiments, the modified oncolytic virus comprises an exogenous CCR5-expressing nucleic acid, which increases the tumor-specific replication of the virus. In some embodiments, the increase in tumor-specific replication is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 10⁶ or even higher folds. Exemplary methods for measuring the increase in viral delivery and spread in tumors include, but are not limited to, fluorescence or bioluminescence based imaging of expression of a reporter gene, quantitative PCR for detection of tumor concentrations of viral genomes or plaque determination of plaque forming units or immunohistochemistry of viral proteins.

In some embodiments, the modified oncolytic virus comprises an exogenous nucleic acid that encodes for a chemokine receptor that is a chimeric protein. At least part of its extracellular domain is from a chemokine receptor that promotes the tumor-targeted delivery of the virus, and at least part of its intracellular domain is from a chemokine receptor that promotes the tumor-specific replication, inhibits immunosuppressive activity, or conveys some other beneficial effects, or vice versa. For instance, the modified oncolytic virus comprises a nucleic acid that encodes for a protein having an intracellular GTPase domain of CCR5 or CXCR3, and an extracellular chemokine-binding domain of CXCR4 or CCR2. In some cases, combining domains with different functionalities achieves further improvement in therapeutic performance of the modified oncolytic virus. In one embodiment of this disclosure, the modified oncolytic virus comprises exogenous nucleic acids that encode for at least one chemokine receptor. In some cases, the modified oncolytic virus comprises exogenous nucleic acids that encode for two or more different chemokine receptors, which are expressed simultaneously by the virus. Exemplary chemokine receptors that are expressed simultaneously from the modified oncolytic viruses described herein include CXCR4 and CCR2. In modified oncolytic viruses expressing more than one chemokine receptors, a combinatorial or synergistic effect against tumor cells is achieved as to the therapeutic application of the oncolytic virus.

### Conditions for Treatment

Provided herein are methods for treatment of cancer including administration of a composition described herein. In some embodiments, the method of treatment is for a hyperproliferative disease. In some embodiments, the hyperproliferative disease is a cancer. In some embodiments, the hyperproliferative disease comprises a tumor. Treatments comprising delivery of a modified oncolytic virus, such as an oncolytic vaccinia virus as described herein, is contemplated. In some embodiments, the cancer is melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, a myeloproliferative neoplasm, or sarcoma.

In some embodiments, compositions described herein are administered to cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer is optionally of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; or hairy cell leukemia. In some cases, solid cancers that are metastatic are treated using the modified oncolytic viruses of this disclosure, such as a modified oncolytic vaccinia virus that is advantageous for systemic delivery. In some cases, solid cancers that are inaccessible or difficult to access, such as for purpose of intratumoral delivery of therapeutic agents, are treated using the modified oncolytic viruses of this disclosure, such as a modified oncolytic vaccinia virus that is advantageous for systemic delivery. In some embodiments, compositions described herein are used to treat cancers that are associated with increased expression of free fatty acids.

This disclosure also contemplates methods for inhibiting or preventing local invasiveness or metastasis, or both, of any type of primary cancer. In exemplary embodiments, the primary cancer is melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the primary cancer is lung cancer. For example, the lung cancer is non-small cell lung carcinoma. Moreover, this disclosure optionally is used to prevent cancer or to treat pre-cancers or premalignant cells, including metaplasias, dysplasias, and hyperplasias. It can also be used to inhibit undesirable but benign cells, such as squamous metaplasia, dysplasia, benign prostate hyperplasia cells, hyperplastic lesions, and the like. In some embodiments, the progression to cancer or to a more severe form of cancer is halted, disrupted, or delayed by methods of this disclosure involving the modified oncolytic virus as discussed herein.

Furthermore, the modified oncolytic virus as disclosed herein is administered for treatment of tumors with high bioavailability of free fatty acids in the tumor microenvironment. In some instances, free fatty acids released by adipocytes in tumors in obese patients feed and enhance the replication of the modified oncolytic virus within the tumor, and formation of EEV form of the virus. The advantage is also be realized in nonobese patients, especially patients who have peritoneal cancer. For example, several peritoneal cancers is targets for therapy using the modified oncolytic viruses of this disclosure as these tend to grow in omentum wall and is fed by adipocytes, and as mentioned above free fatty acids released by adipocytes in tumors feed and enhance the replication of the modified oncolytic virus within the tumor. The modified oncolytic virus as disclosed herein forms an increased titer of extracellular enveloped virus (EEV) in tumors with high bioavailability of free fatty acids.

Provided herein are methods for treating a subject by administration of one or more modified oncolytic viruses, as disclosed herein. An “individual” or “subject,” as used interchangeably herein, refers to a human or a non-human subject. Non-limiting examples of non-human subjects include non-human primates, dogs, cats, mice, rats, guinea pigs, rabbits, pigs, fowl, horses, cows, goats, sheep, cetaceans, etc. In some embodiments, the subject is human.

Provided are methods for producing a toxic effect in a cancer cell comprising administering, to the cancer cell, a therapeutically effective amount of a modified virus, such as an oncolytic vaccinia virus, as described above, or a pharmaceutical composition containing the same. This disclosure further provides a method of inhibiting at least one of growth and proliferation of a second cancer cell comprising administering, to a first cancer cell, a modified oncolytic virus as described above such that the first cancer cell is infected with said virus. Thus, in some embodiments of the methods disclosed here, it is contemplated that not every cancer or tumor cell is infected upon administering a therapeutically effective amount of an oncolytic vaccinia virus, as described herein, or a pharmaceutical composition containing the same, and growth of non-infected cells is inhibited without direct infection.

In some examples, to induce oncolysis, kill cells, inhibit growth, inhibit metastases, decrease tumor size, and otherwise reverse or reduce the malignant phenotype of tumor cells, using the methods and compositions of the present disclosure, a cancer cell or a tumor is contacted with a therapeutically effective dose of an exemplary oncolytic vaccinia virus as described herein or a pharmaceutical composition containing the same. In certain embodiments, an effective amount of a modified oncolytic virus of the present disclosure, such as an oncolytic vaccinia virus as described herein or a pharmaceutical composition thereof, can include an amount sufficient to induce oncolysis, the disruption or lysis of a cancer cell or the inhibition or reduction in the growth or size of a cancer cell. Reducing the growth of a cancer cell is manifested, for example, by cell death or a slower replication rate or reduced growth rate of a tumor comprising the cell or a prolonged survival of a subject containing the cancer cell.

In some embodiments, use of a modified virus as described herein inhibits growth of a tumor by about 10%, by about 20%, by about 30%, by about 40%, by about 50%, by about 60%, by about 70%, by about 80%, by about 90%, by about 95%, by about 99%, or by about 100% as compared to an untreated tumor.

In some embodiments, use of a modified virus as described herein reduces the size of a tumor by about 10%, by about 20%, by about 30%, by about 40%, by about 50%, by about 60%, by about 70%, by about 80%, by about 90%, by about 95%, by about 99%, or by about 100% as compared to an untreated tumor.

Provided herein are methods for treating a subject having a cancer or a tumor comprising administering, to the subject, an effective amount of a modified virus, as described above. An effective amount in such method includes an amount that reduces growth rate or spread of the cancer or that prolongs survival in the subject. This disclosure provides a method of reducing the growth of a tumor, which method comprises administering, to the tumor, an effective amount of a modified oncolytic virus as described above. In certain embodiments, an effective amount of a modified virus, or a pharmaceutical composition thereof, includes an amount sufficient to induce the slowing, inhibition or reduction in the growth or size of a tumor and includes the eradication of the tumor. Reducing the growth of a tumor is manifested, for example, by reduced growth rate or a prolonged survival of a subject containing the tumor. In certain embodiments, an effective amount of a modified virus, or a pharmaceutical composition thereof, includes an amount sufficient to activate an anti-tumor response. In some embodiments, activating an anti-tumor response includes activating T-cells. In certain embodiments, an effective amount of a modified virus, or a pharmaceutical composition thereof, includes an amount sufficient to reduce incidence of tumor growth. In some embodiments, reducing incidence of tumor growth includes suppression of metastasis, prevention of primary tumor growth, suppression of existing tumor growth, or any combination thereof.

Provided herein are methods for determining the infectivity or anti-tumor activity, or amount of tumor specific viral replication of an oncolytic vaccinia virus as described herein, which method comprises; (i) administering to a subject a therapeutically effective amount of an oncolytic vaccinia virus or a pharmaceutical composition according to the present disclosure, which further expresses a luciferase reporter gene, alone or in combination with a further therapy; (ii) collecting a first biological sample from the subject immediately after administering the virus and determining the level of the luciferase reporter in the first biological sample (iii) collecting a second biological sample from the subject following the administration in step (ii) and detecting the level of the luciferase reporter in the second biological sample, wherein the oncolytic vaccinia virus is determined to be infective, demonstrate anti-tumor activity, exhibit tumor specific viral replication if the level of luciferase is higher in step (iii) than in step (ii). The second biological sample is collected about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i). In some embodiments, the method of mentioned above further comprises, detecting in steps (ii) and (iii), the level of one or more assaying cytokine levels, e.g., IL-2, IL-7, IL-8, IL-10, IFN-γ, GM-CSF, TNF-α, IL-6, IL-4, IL-5, and IL-13, in plasma samples collected from a subject after administering to said subject a therapeutically effective amount of a modified oncolytic virus of the present disclosure, such as an oncolytic vaccinia virus as described herein or a pharmaceutical composition comprising the same. In some embodiments of this disclosure, the increase in luciferase bioluminescence between steps (ii) and (iii) mentioned above is higher for a modified oncolytic virus as described herein, compared to that in an otherwise identical virus that does not comprise the modifications in the modified oncolytic virus. Other exemplary techniques for detecting and monitoring viral load after administration of the modified oncolytic viruses include real-time quantitative PCR.

Provided herein are methods for monitoring the pharmacokinetics following administration of a therapeutically effective amount of modified oncolytic viruses according to the present disclosure, such as oncolytic vaccinia virus or a pharmaceutical composition containing the vaccinia virus, as described herein. An exemplary method for monitoring the pharmacokinetics comprises the following steps: (i) administering to the subject a therapeutically effective amount of an oncolytic vaccinia virus or a pharmaceutical composition comprising the same, alone or in combination with a further therapy; (ii) collecting biological samples from the subject at one or more time points selected from about 15 minutes, about 30 minutes, about 45 mins, about 60 mins, about 75 mins, about 90 mins, about 120 mins, about 180 mins, and about 240 mins, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i); and (iii) detecting the quantity of the viral genome (or a reporter gene inserted within the viral genome, such as luciferase) in the biological samples collected at the above mentioned time points. In some instances, viral genome copies/mL is highest in the sample collected at the 15 mins time point and further the sample collected at the 240 mins time point does not contain a detectable quantity of the viral genome. Therefore, in some instances, a viral peak is observed at about 15 mins following administration and majority of the viruses is cleared from the subject's system after about 240 mins (or 4 hours). In some instances, a first viral peak is observed after about 15 mins following administration and a second viral peak is observed in the biological samples collected in the subsequent time points, e.g., at about 30 mins, about 45 mins, about 60 mins, or about 90 mins. The biological sample is, in exemplar embodiments, blood, and the quantity of viral genome/mL is determined by quantitative PCR or other appropriate techniques. In some examples, a first viral peak is observed after about 15 mins following administration and a second viral peak is observed after about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months following administration of a modified oncolytic virus of the present disclosure, such as an oncolytic vaccinia virus as described herein.

In some instances, tumor-selective replication of a modified virus, such as an oncolytic vaccinia virus is measured through use of a reporter gene, such as a luciferase gene. In some embodiments, the luciferase gene is inserted into the genome of a virus, and a tumor cell is infected with the virus. Bioluminescence in infected tumor cells is measured to monitor tumor-selective replication. Some examples show an increase in luciferase reporter bioluminescence in a modified oncolytic virus of this disclosure, compared to that in an otherwise identical oncolytic vaccinia virus that does not contain the modifications in the modified oncolytic virus.

Provided herein are methods for delivering modified viruses as described herein. Modified viruses provided herein are capable of increased replication in tumor cells compared to normal cells. In some embodiments, a modified virus produces about 2-fold, about 3-fold, about 4-fold, about 5-fold, about 6-fold, about 7-fold, about 8-fold, about 9-fold, about 10-fold, about 12-fold, about 14-fold, about 16-fold, about 18-fold, about 20-fold, about 30-fold, about 40-fold, or about 50-fold more copies/mg in tumor cells compared to production in normal cells.

Provided herein are methods of contacting a tumor with a modified virus as described herein. Modified viruses provided herein can increase infiltration of CD3+CD8+ T cells in to the tumor as compared to an untreated tumor. In some embodiments, contacting a tumor with a modified virus as described herein results in about 10%, about 20%, about 30%, about 40%, about 50%, about 60%, about 70%, about 80%, about 90%, or about 100% increase in CD3+CD8+ T cells in the tumor as compared to an untreated tumor. In some embodiments, contacting a tumor with a modified virus as described herein results in about a 2-fold, about a 3-fold, about a 4-fold, about a 5-fold, about a 6-fold, about a 7-fold, about an 8-fold, about a 9-fold, or about a 10-fold increase in CD3+CD8+ T cells in the tumor as compared to an untreated tumor.

### Dosage

In some embodiments, the amount of a modified oncolytic virus described herein administered to a subject is between about 10³ and 10¹² infectious viral particles or plaque forming units (PFU), or between about 10⁵ and 10¹⁰ PFU, or between about 10⁵ and 10⁸ PFU, or between about 10⁸ and 10¹⁰ PFU. In some embodiments, the amount of a modified oncolytic virus of this disclosure administered to a subject is between about 10³ and 10¹² viral particles or plaque forming units (PFU), or between about 10⁵ and 10¹⁰ PFU, or between about 10⁵ and 10⁸ PFU, or between about 10⁸ and 10¹⁰ PFU. In some embodiments, a modified oncolytic virus of this disclosure is administered at a dose that comprises about 10³ PFU/dose to about 10⁴ PFU/dose, about 10⁴ PFU/dose to about 10⁵ PFU/dose, about 10⁵ PFU/dose to about 10⁶ PFU/dose, about 10⁷ PFU/dose to about 10⁸ PFU/dose, about 10⁹ PFU/dose to about 10¹⁰ PFU/dose, about 10¹⁰ PFU/dose to about 10¹¹ PFU/dose, about 10¹¹ PFU/dose to about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, a modified oncolytic virus of this disclosure is administered at a dose that comprises about 2×10³ PFU/dose, 3×10³ PFU/dose, 4×10³ PFU/dose, 5×10³ PFU/dose, 6×10³ PFU/dose, 7×10³ PFU/dose, 8×10³ PFU/dose, 9×10³ PFU/dose, about 10⁴ PFU/dose, about 2×10⁴ PFU/dose, about 3×10⁴ PFU/dose, about 4×10⁴ PFU/dose, about 5×10⁴ PFU/dose, about 6×10⁴ PFU/dose, about 7×10⁴ PFU/dose, about 8×10⁴ PFU/dose, about 9×10⁴ PFU/dose, about 10⁵ PFU/dose, 2×10⁵ PFU/dose, 3×10⁵ PFU/dose, 4×10⁵ PFU/dose, 5×10⁵ PFU/dose, 6×10⁵ PFU/dose, 7×10⁵ PFU/dose, 8×10⁵ PFU/dose, 9×10⁵ PFU/dose, about 10⁶ PFU/dose, about 2×10⁶ PFU/dose, about 3×10⁶ PFU/dose, about 4×10⁶ PFU/dose, about 5×10⁶ PFU/dose, about 6×10⁶ PFU/dose, about 7×10⁶ PFU/dose, about 8×10⁶ PFU/dose, about 9×10⁶ PFU/dose, about 10⁷ PFU/dose, about 2×10⁷ PFU/dose, about 3×10⁷ PFU/dose, about 4×10⁷ PFU/dose, about 5×10⁷ PFU/dose, about 6×10⁷ PFU/dose, about 7×10⁷ PFU/dose, about 8×10⁷ PFU/dose, about 9×10⁷ PFU/dose, about 10⁸ PFU/dose, about 2×10⁸ PFU/dose, about 3×10⁸ PFU/dose, about 4×10⁸ PFU/dose, about 5×10⁸ PFU/dose, about 6×10⁸ PFU/dose, about 7×10⁸ PFU/dose, about 8×10⁸ PFU/dose, about 9×10⁸ PFU/dose, about 10° PFU/dose, about 2×10⁹ PFU/dose, about 3×10⁹ PFU/dose, about 4×10⁹ PFU/dose, about 5×10⁹ PFU/dose, about 6×10⁹ PFU/dose, about 7×10⁹ PFU/dose, about 8×10⁹ PFU/dose, about 9×10⁹ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹¹ PFU/dose, about 2×10¹¹ PFU/dose, about 3×10¹¹ PFU/dose, about 4×10¹¹ PFU/dose, about 5×10¹¹ PFU/dose, about 6×10¹¹ PFU/dose, about 7×10¹¹ PFU/dose, about 8×10¹¹ PFU/dose, about 9×10¹¹ PFU/dose, or about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, a modified oncolytic virus of this disclosure is administered at a dose that comprises 5×10⁹ PFU/dose. In some embodiments, a modified oncolytic virus of this disclosure is administered at a dose that comprises up to 5×10⁹ PFU/dose.

In some embodiments, a modified oncolytic virus of this disclosure is administered at a dose that comprises about 10³ viral particles/dose to about 10⁴ viral particles/dose, about 10⁴ viral particles/dose to about 10⁵ viral particles/dose, about 10⁵ viral particles/dose to about 10⁶ viral particles/dose, about 10⁷ viral particles/dose to about 10⁸ viral particles/dose, about 10⁹ viral particles/dose to about 10¹⁰ viral particles/dose, about 10¹⁰ viral particles/dose to about 10¹¹ viral particles/dose, about 10¹¹ viral particles/dose to about 10¹² viral particles/dose, about 10¹² viral particles/dose to about 10¹³ viral particles/dose, about 10¹³ viral particles/dose to about 10¹⁴ viral particles/dose, or about 10¹⁴ viral particles/dose to about 10¹⁵ viral particles/dose.

In some embodiments, a modified oncolytic virus of this disclosure is administered at a dose that comprises about 10³ PFU/kg to about 10⁴ PFU/kg, about 10⁴ PFU/kg to about 10⁵ PFU/kg, about 10⁵ PFU/kg to about 10⁶ PFU/kg, about 10⁷ PFU/kg to about 10⁸ PFU/kg, about 10⁹ PFU/kg to about 10¹⁰ PFU/kg, about 10¹⁰ PFU/kg to about 10¹¹ PFU/kg, about 10¹¹ PFU/kg to about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic virus of this disclosure is administered at a dose that comprises about 2×10³ PFU/kg, 3×10³ PFU/kg, 4×10³ PFU/kg, 5×10³ PFU/kg, 6×10³ PFU/kg, 7×10³ PFU/kg, 8×10³ PFU/kg, 9×10³ PFU/kg, about 10⁴ PFU/kg, about 2×10⁴ PFU/kg, about 3×10⁴ PFU/kg, about 4×10⁴ PFU/kg, about 5×10⁴ PFU/kg, about 6×10⁴ PFU/kg, about 7×10⁴ PFU/kg, about 8×10⁴ PFU/kg, about 9×10⁴ PFU/kg, about 10⁵ PFU/kg, 2×10⁵ PFU/kg, 3×10⁵ PFU/kg, 4×10⁵ PFU/kg, 5×10⁵ PFU/kg, 6×10⁵ PFU/kg, 7×10⁵ PFU/kg, 8×10⁵ PFU/kg, 9×10⁵ PFU/kg, about 10⁶ PFU/kg, about 2×10⁶ PFU/kg, about 3×10⁶ PFU/kg, about 4×10⁶ PFU/kg, about 5×10⁶ PFU/kg, about 6×10⁶ PFU/kg, about 7×10⁶ PFU/kg, about 8×10⁶ PFU/kg, about 9×10⁶ PFU/kg, about 10⁷ PFU/kg, about 2×10⁷ PFU/kg, about 3×10⁷ PFU/kg, about 4×10⁷ PFU/kg, about 5×10⁷ PFU/kg, about 6×10⁷ PFU/kg, about 7×10⁷ PFU/kg, about 8×10⁷ PFU/kg, about 9×10⁷ PFU/kg, about 10⁸ PFU/kg, about 2×10⁸ PFU/kg, about 3×10⁸ PFU/kg, about 4×10⁸ PFU/kg, about 5×10⁸ PFU/kg, about 6×10⁸ PFU/kg, about 7×10⁸ PFU/kg, about 8×10⁸ PFU/kg, about 9×10⁸ PFU/kg, about 10⁹ PFU/kg, about 2×10⁹ PFU/kg, about 3×10⁹ PFU/kg, about 4×10⁹ PFU/kg, about 5×10⁹ PFU/kg, about 6×10⁹ PFU/kg, about 7×10⁹ PFU/kg, about 8×10⁹ PFU/kg, about 9×10⁹ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹¹ PFU/kg, about 2×10¹¹ PFU/kg, about 3×10¹¹ PFU/kg, about 4×10¹¹ PFU/kg, about 5×10¹¹ PFU/kg, about 6×10¹¹ PFU/kg, about 7×10¹¹ PFU/kg, about 8×10¹¹ PFU/kg, about 9×10¹¹ PFU/kg, or about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic virus of this disclosure is administered at a dose that comprises 5×10⁹ PFU/kg. In some embodiments, a modified oncolytic virus of this disclosure is administered at a dose that comprises up to 5×10⁹ PFU/kg.

In some embodiments, a modified oncolytic virus of this disclosure is administered at a dose that comprises about 10³ viral particles/kg to about 10⁴ viral particles/kg, about 10⁴ viral particles/kg to about 10⁵ viral particles/kg, about 10⁵ viral particles/kg to about 10⁶ viral particles/kg, about 10⁷ viral particles/kg to about 10⁸ viral particles/kg, about 10⁹ viral particles/kg to about 10¹⁰ viral particles/kg, about 10¹⁰ viral particles/kg to about 10¹¹ viral particles/kg, about 10¹¹ viral particles/kg to about 10¹² viral particles/kg, about 10¹² viral particles/kg to about 10¹³ viral particles/kg, about 10¹³ viral particles/kg to about 10¹⁴ viral particles/kg, or about 10¹⁴ viral particles/kg to about 10¹⁵ viral particles/kg.

A liquid dosage form of an oncolytic vaccinia virus as described herein comprises, in certain embodiments, a viral dose of about 10³ PFU/mL to about 10⁴ PFU/mL, about 10⁴ PFU/mL to about 10⁵ PFU/mL, about 10⁵ PFU/mL to about 10⁶ PFU/mL, about 10⁷ PFU/mL to about 10⁸ PFU/mL, about 10⁹ PFU/mL to about 10¹⁰ PFU/mL, about 10¹⁰ PFU/mL to about 10¹¹ PFU/mL, about 10¹¹ PFU/mL to about 10¹² PFU/mL, about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, a modified oncolytic virus of this disclosure is administered at a dose that comprises about 2×10³ PFU/mL, 3×10³ PFU/mL, 4×10³ PFU/mL, 5×10³ PFU/mL, 6×10³ PFU/mL, 7×10³ PFU/mL, 8×10³ PFU/mL, 9×10³ PFU/mL, about 10⁴ PFU/mL, about 2×10⁴ PFU/mL, about 3×10⁴ PFU/mL, about 4×10⁴ PFU/mL, about 5×10⁴ PFU/mL, about 6×10⁴ PFU/mL, about 7×10⁴ PFU/mL, about 8×10⁴ PFU/mL, about 9×10⁴ PFU/mL, about 10⁵ PFU/mL, 2×10⁵ PFU/mL, 3×10⁵ PFU/mL, 4×10⁵ PFU/mL, 5×10⁵ PFU/mL, 6×10⁵ PFU/mL, 7×10⁵ PFU/mL, 8×10⁵ PFU/mL, 9×10⁵ PFU/mL, about 10⁶ PFU/mL, about 2×10⁶ PFU/mL, about 3×10⁶ PFU/mL, about 4×10⁶ PFU/mL, about 5×10⁶ PFU/mL, about 6×10⁶ PFU/mL, about 7×10⁶ PFU/mL, about 8×10⁶ PFU/mL, about 9×10⁶ PFU/mL, about 10⁷ PFU/mL, about 2×10⁷ PFU/mL, about 3×10⁷ PFU/mL, about 4×10⁷ PFU/mL, about 5×10⁷ PFU/mL, about 6×10⁷ PFU/mL, about 7×10⁷ PFU/mL, about 8×10⁷ PFU/mL, about 9×10⁷ PFU/mL, about 10⁸ PFU/mL, about 2×10⁸ PFU/mL, about 3×10⁸ PFU/mL, about 4×10⁸ PFU/mL, about 5×10⁸ PFU/mL, about 6×10⁸ PFU/mL, about 7×10⁸ PFU/mL, about 8×10⁸ PFU/mL, about 9×10⁸ PFU/mL, about 10⁹ PFU/mL, about 2×10⁹ PFU/mL, about 3×10⁹ PFU/mL, about 4×10⁹ PFU/mL, about 5×10⁹ PFU/mL, about 6×10⁹ PFU/mL, about 7×10⁹ PFU/mL, about 8×10⁹ PFU/mL, about 9×10⁹ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹¹ PFU/mL, about 2×10¹¹ PFU/mL, about 3×10¹¹ PFU/mL, about 4×10¹¹ PFU/mL, about 5×10¹¹ PFU/mL, about 6×10¹¹ PFU/mL, about 7×10¹¹ PFU/mL, about 8×10¹¹ PFU/mL, about 9×10¹¹ PFU/mL, or about 10¹² PFU/mL, about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, a modified oncolytic virus of this disclosure is administered at a dose that comprises 5×10⁹ PFU/mL. In some embodiments, a modified oncolytic virus of this disclosure is administered at a dose that comprises up to 5×10⁹ PFU/mL.

In some instances, where the modified oncolytic virus is administered by an injection, the dosage comprises about 10³ viral particles per injection, 10⁴ viral particles per injection, 10⁵ viral particles per injection, 10⁶ viral particles per injection, 10⁷ viral particles per injection, 10⁸ viral particles per injection, 10⁹ viral particles per injection, 10¹⁰ viral particles per injection, 10¹¹ viral particles per injection, 10¹² viral particles per injection, 2×10¹² viral particles per injection, 10¹³ viral particles per injection, 10¹⁴ viral particles per injection, or 10¹⁵ viral particles per injection. In further instances, where the modified oncolytic virus is administered by an injection, the dosage comprises about 10³ infectious viral particles per injection, 10⁴ infectious viral particles per injection, 10⁵ infectious viral particles per injection, 10⁶ infectious viral particles per injection, 10⁷ infectious viral particles per injection, 10⁸ infectious viral particles per injection, 10⁹ infectious viral particles per injection, 10¹⁰ infectious viral particles per injection, 10¹¹ infectious viral particles per injection, 10¹² infectious viral particles per injection, 2×10¹² infectious viral particles per injection, 10¹³ infectious viral particles per injection, 10¹⁴ infectious viral particles per injection, or 10¹⁵ infectious viral particles per injection. In certain embodiments, the virus is administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor. In certain embodiments, a single dose of virus refers to the amount administered to a subject or a tumor over a 1, 2, 5, 10, 15, 20 or 24 hour period. In certain embodiments, the dose is spread over time or by separate injection. In certain embodiments, multiple doses (e.g., 2, 3, 4, 5, 6 or more doses) of the vaccinia virus is administered to the subject, for example, where a second treatment occurs within 1, 2, 3, 4, 5, 6, 7 days or weeks of a first treatment. In certain embodiments, multiple doses of the modified oncolytic virus is administered to the subject over a period of 1, 2, 3, 4, 5, 6, 7 or more days or weeks. In certain embodiments, the oncolytic virus or the pharmaceutical composition as described herein is administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the oncolytic vaccinia virus or the pharmaceutical composition as described herein is, in certain instances, once daily, twice daily, once every week, once every three weeks, once every four weeks (or once a month), once every 8 weeks (or once every 2 months), once every 12 weeks (or once every 3 months), or once every 24 weeks (once every 6 months). In some embodiments of the methods disclosed herein, the oncolytic vaccinia virus or the pharmaceutical composition is administered, independently, in an initial dose for a first period of time, an intermediate dose for a second period of time, and a high dose for a third period of time. In some embodiments, the initial dose is lower than the intermediate dose and the intermediate dose is lower than the high dose. In some embodiments, the first, second, and third periods of time are, independently, about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer.

In some examples, the subject is put on a reduced carbohydrate diet, e.g., a ketogenic diet prior to, concurrent with, and following administration of the modified oncolytic viruses, such as the oncolytic vaccinia viruses or the pharmaceutical composition comprising the same, as described herein, according to any of the methods of treatment described herein. In certain embodiments, the subject is put on a diet that comprises consuming less than 500 grams of carbohydrates per day, less than 450 grams of carbohydrates per day, less than 450 grams of carbohydrates per day, less than 400 grams of carbohydrates per day, less than 350 grams of carbohydrates per day, less than 300 grams of carbohydrates per day, less than 250 grams of carbohydrates per day, less than 200 grams of carbohydrates per day, less than 150 grams of carbohydrates per day, less than 100 grams of carbohydrates per day, less than 90 grams of carbohydrates per day, less than 80 grams of carbohydrates per day, less than 70 grams of carbohydrates per day, less than 60 grams of carbohydrates per day, less than 50 grams of carbohydrates per day, less than 40 grams of carbohydrates per day, less than 30 grams of carbohydrates per day, less than 20 grams of carbohydrates per day, less or than 10 grams of carbohydrates per day.

An exemplary method for the delivery of a modified oncolytic virus of the present disclosure, such as an oncolytic vaccinia virus as described herein or a pharmaceutical composition comprising the same, to cancer or tumor cells is via intratumoral injection. However, alternate methods of administration are also used, e.g., intravenous, via infusion, parenteral, intravenous, intradermal, intramuscular, transdermal, rectal, intraurethral, intravaginal, intranasal, intrathecal, or intraperitoneal. The routes of administration vary with the location and nature of the tumor. In certain embodiments, the route of administration is intradental, transdermal, parenteral, intraperitoneal, intravenous, intramuscular, intranasal, subcutaneous, regional (e.g., in the proximity of a tumor, particularly with the vasculature or adjacent vasculature of a tumor), percutaneous, intrathecal, intratracheal, intraperitoneal, intraarterial, intravesical, intratumoral, inhalation, perfusion, by lavage or orally. An injectable dose of the oncolytic virus is administered as a bolus injection or as a slow infusion. In certain embodiments, the modified oncolytic virus is administered to the patient from a source implanted in the patient. In certain embodiments, administration of the modified oncolytic virus occurs by continuous infusion over a selected period of time. In some instances, an oncolytic vaccinia virus as described herein, or a pharmaceutical composition containing the same is administered at a therapeutically effective dose by infusion over a period of about 15 mins, about 30 mins, about 45 mins, about 50 mins, about 55 mins, about 60 minutes, about 75 mins, about 90 mins, about 100 mins, or about 120 mins or longer. The oncolytic virus or the pharmaceutical composition of the present disclosure is administered as a liquid dosage, wherein the total volume of administration is about 1 mL to about 5 mL, about 5 mL to 10 mL, about 15 mL to about 20 mL, about 25 mL to about 30 mL, about 30 mL to about 50 mL, about 50 mL to about 100 mL, about 100 mL to 150 mL, about 150 mL to about 200 mL, about 200 mL to about 250 mL, about 250 mL to about 300 mL, about 300 mL to about 350 mL, about 350 mL to about 400 mL, about 400 mL to about 450 mL, about 450 mL to 500 mL, about 500 mL to 750 mL, or about 750 mL to 1000 mL.

### Formulations

Pharmaceutical compositions containing a modified virus, such as an oncolytic vaccinia virus, as described herein, are prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, a pharmaceutical composition as described herein comprises a stabilizer and a buffer. In some embodiments, a pharmaceutical composition as described herein can comprise a solubilizer, such as sterile water, Tris-buffer. In some embodiments, a pharmaceutical composition as described herein can comprise an excipient. Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In certain embodiments, a buffering agent includes phosphate buffered saline (PBS), Dulbecco's PBS (DPBS), TRIS-buffered saline (TBS), Hank's balanced salt solution (HBSS), Earle's balanced salt solution (EBSS), standard saline citrate (SSC), HEPES-buffered saline (HBS), or Gey's balanced salt solution.

In certain embodiments, a pharmaceutical composition of this disclosure comprises an effective amount of a modified virus, disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers include gels, bioadsorbable matrix materials, implantation elements containing the modified oncolytic virus or any other suitable vehicle, delivery or dispensing means or material. Such carriers are formulated by conventional methods and are administered to the subject at an effective amount.

### Methods of Production

The modified oncolytic viruses of this disclosure are produced by methods known to one of skill in the art. In certain embodiments, the modified oncolytic virus is propagated in suitable host cells, e.g., HeLa cells, 293 cells, or Vero cells, isolated from host cells and stored in conditions that promote stability and integrity of the virus, such that loss of infectivity over time is minimized. In certain exemplary methods, the modified oncolytic viruses are propagated in host cells using cell stacks, roller bottles, or perfusion bioreactors. In some examples, downstream methods for purification of the modified oncolytic viruses comprise filtration (e.g., depth filtration, tangential flow filtration, or a combination thereof), ultracentrifugation, chromatographic capture, or any combination thereof. The modified oncolytic virus is stored, e.g., by freezing or drying, such as by lyophilization. In certain embodiments, prior to administration, the stored modified oncolytic virus is reconstituted (if dried for storage) and diluted in a pharmaceutically acceptable carrier for administration.

Some embodiments provide that the modified oncolytic virus as described herein, exhibit a higher titer in HeLa cells and 293 cells compared to an otherwise identical virus that does not comprise the modifications in the modified oncolytic virus. In certain instances, a higher titer in HeLa cells and 293 cells is seen in modified oncolytic virus.

### Kits

In embodiments, this disclosure provides for a kit for administering a modified oncolytic virus as described herein. In certain embodiments, a kit of this disclosure includes a modified oncolytic virus or a pharmaceutical composition comprising a modified oncolytic virus as described above. In certain embodiments, a kit of this disclosure further includes one or more components such as instructions for use, devices and additional reagents, and components, such as tubes, containers, and syringes for performing the methods disclosed above. In certain embodiments, a kit of this disclosure further includes one or more agents, e.g., at least one of an anti-cancer agent, an immunomodulatory agent, or any combinations thereof, that is administered in combination with a modified virus.

In certain embodiments, a kit of this disclosure comprises one or more containers containing a modified virus, disclosed herein. For example, and not by way of limitation, a kit of this disclosure comprises one or more containers that contain a modified oncolytic virus of this disclosure.

In certain embodiments, a kit of this disclosure includes instructions for use, a device for administering the modified oncolytic virus to a subject, or a device for administering an additional agent or compound to a subject. For example, and not by way of limitation, the instructions include a description of the modified oncolytic virus and, optionally, other components included in the kit, and methods for administration, including methods for determining the proper state of the subject, the proper dosage amount and the proper administration method for administering the modified virus. Instructions also optionally include guidance for monitoring the subject over duration of the treatment time.

In certain embodiments, a kit of this disclosure includes a device for administering the modified oncolytic virus to a subject. Any of a variety of devices known in the art for administering medications and pharmaceutical compositions is included in the kits provided herein. For example, and not by way of limitation, such devices include, a hypodermic needle, an intravenous needle, a catheter, a needle-less injection device, an inhaler, and a liquid dispenser, such as an eyedropper. In certain embodiments, a modified oncolytic virus to be delivered systemically, for example, by intravenous injection, an intratumoral injection, an intraperitoneal injection, is included in a kit with a hypodermic needle and syringe.

While preferred embodiments of this disclosure have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments of this disclosure described herein are employed in practicing the disclosure. It is intended that the following claims define the scope of the disclosure and that methods and structures within the scope of these claims and their equivalents be covered thereby.

### Exemplary Embodiments

Provided herein are compositions, wherein the compositions comprise a vector, wherein the vector comprises: an exogenous nucleic acid comprising a sequence encoding for a cytokine or a functional variant thereof; an exogenous nucleic acid comprising a sequence encoding for a chemokine receptor or a functional variant thereof; and a first promoter region, wherein the first promoter region is upstream to the sequence encoding for the chemokine receptor and provides for expression of the chemokine receptor prior to expression of the cytokine. Further provided herein are compositions, wherein the encodedchemokine receptor comprises at least one of: a CXC receptor, a CC receptor, a CX3C receptor, an XC receptor, a functional fragment thereof, a functional variant thereof, or any combinations thereof. Further provided herein are compositions, wherein the encoded chemokine receptor comprises at least one of: CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, a functional fragment thereof or a functional variant thereof, or any combinations thereof. Further provided herein are compositions, wherein the encoded chemokine receptor is CXCR3. Further provided herein are compositions, wherein the encoded chemokine receptor comprises an amino acid sequence having at least 85%, 90%, 95%, or 99% sequence identity to SEQ ID NO: 42 or SEQ ID NO: 43. Further provided herein are compositions, wherein the encoded chemokine receptor comprises an amino acid sequence as set forth in SEQ ID NO: 42 or SEQ ID NO: 43. Further provided herein are compositions, wherein the first promoter region comprises an early promoter. Further provided herein are compositions, wherein the early promoter comprises any one of A52R, pB8, mH5, I4L, LEO, pF11, I3L, P7.5, TK promoter, F7L, H5R, a short synthetic promoter (SSP) or any variation or combination thereof. Further provided herein are compositions, wherein the early promoter comprises the A52R promoter. Further provided herein are compositions, wherein the encoded cytokine comprises IL-12 or a functional variant thereof. Further provided herein are compositions, wherein the encoded IL-12 is a murine IL-12 or a human IL-12. Further provided herein are compositions, wherein the encoded IL-12 comprises an alpha subunit and a beta subunit. Further provided herein are compositions, wherein sequence encoding the IL-12 alpha subunit and the IL-12 beta subunit further comprises a sequence encoding a linker. Further provided herein are compositions, wherein the encoded IL-12 alpha subunit comprises an amino acid sequence having at least 85%, 90%, 95%, or 99% sequence identity to either one of SEQ ID NO: 13 or SEQ ID NO: 16. Further provided herein are compositions, wherein the encoded IL-12 alpha subunit comprises an amino acid sequence as set forth in either one of SEQ ID NO: 13 or SEQ ID NO: 16. Further provided herein are compositions, wherein the encoded IL-12 beta subunit comprises an amino acid sequence having at least 85%, 90%, 95%, or 99% sequence identity to either one of SEQ ID NO: 14 or SEQ ID NO: 17. Further provided herein are compositions, wherein the IL-12 beta subunit comprises an amino acid sequence as set forth in either one of SEQ ID NO: 14 or SEQ ID NO: 17. Further provided herein are compositions, wherein the encoded linker comprises an amino acid sequence having at least 85%, 90%, 95%, or 99% sequence identity to SEQ ID NO: 18. Further provided herein are compositions, wherein the encoded linker comprises an amino acid sequence as set forth in SEQ ID NO: 18. Further provided herein are compositions, wherein the encoded IL-12 comprises an amino acid sequence having at least 85%, 90%, 95%, or 99% sequence identity to either one of SEQ ID NO: 12 or SEQ ID NO: 15. Further provided herein are compositions, wherein encoded the IL-12 comprises an amino acid sequence as set forth in either one of SEQ ID NO: 12 or SEQ ID NO: 15. Further provided herein are compositions, wherein the sequence encoding for the IL-12 comprises a nucleic acid sequence having at least 85%, 90%, 95%, or 99% sequence identity to either one of SEQ ID NO: 64 or SEQ ID NO: 66. Further provided herein are compositions, wherein the sequence encoding the IL-12 comprises a nucleic acid sequence as set forth in either one of SEQ ID NO: 64 or SEQ ID NO: 66. Further provided herein are compositions, further comprising an exogenous nucleic acid comprising a sequence encoding for a Transforming growth factor beta (TGF-beta) activity inhibitor; wherein the first promoter region provides for expression of the chemokine receptor prior to expression of the TGF-beta activity inhibitor. Further provided herein are compositions, wherein the encoded TGF-beta activity inhibitor comprises a TGF-beta dominant negative, a TGF-beta receptor dominant negative, a protein that binds TGF-beta, or a protein that binds a TGF-beta receptor. Further provided herein are compositions, wherein the encoded protein that binds TGF-beta receptor is a protein comprising a domain of TGF-beta. Further provided herein are compositions, wherein the encoded protein comprising a domain of TGF-beta comprises an amino acid sequence as set forth in any one of SEQ ID NOs: 1-9. Further provided herein are compositions, wherein the encoded protein comprising a domain of TGF-beta comprises an amino acid sequence having at least 85%, 90%, 95%, or 99% sequence identity to either one of SEQ ID NO: 7 or SEQ ID NO: 8. Further provided herein are compositions, wherein the encoded protein comprising a domain of TGF-beta comprises an amino acid sequence as set forth in either one of SEQ ID NO: 7 or SEQ ID NO: 8. Further provided herein are compositions, wherein the sequence encoding for the Transforming growth factor beta (TGF-beta) activity inhibitor comprises a nucleic acid sequence having at least 85%, 90%, 95%, or 99% sequence identity to SEQ ID NO: 67. Further provided herein are compositions, wherein the sequence encoding for the TGF-beta activity inhibitor comprises a nucleic acid sequence as set forth in SEQ ID NO: 67. Further provided herein are compositions, wherein the protein that binds to TGF-beta receptor is a TGF-beta fusion protein. Further provided herein are compositions, wherein the encoded TGF-beta activity inhibitor comprises the protein that binds to TGF-beta receptor, and wherein the protein that binds to TGF-beta receptor comprises a TGF-beta fusion 1 (TGFbf1) protein. Further provided herein are compositions, wherein the TGFbf1 protein comprises a murine IL-2 signal peptide and a TGF-beta variant 1 (TGFbv1). Further provided herein are compositions, wherein the murine IL-2 signal peptide comprises an amino acid sequence having at least 85%, 90%, 95%, or 99% sequence identity to SEQ ID NO: 36. Further provided herein are compositions, wherein the murine IL-2 signal peptide comprises an amino acid sequence as set forth in SEQ ID NO: 36. Further provided herein are compositions, wherein the TGFbv1 comprises an amino acid sequence having at least 85%, 90%, 95%, or 99% sequence identity to SEQ ID NO: 7. Further provided herein are compositions, wherein the TGFbv1 comprises a sequence as set forth in SEQ ID NO: 7. Further provided herein are compositions, wherein the encoded TGF-beta activity inhibitor comprises the protein that binds to TGF-beta receptor, and wherein the protein that binds to TGF-beta receptor comprises a TGF-beta fusion 2 (TGFbf2) protein. Further provided herein are compositions, wherein the TGFbf2 comprises a human IgE signal peptide and a TGF-beta variant 2 (TGFbv2). Further provided herein are compositions, wherein the human IgE signal peptide comprises an amino acid sequence having at least 85%, 90%, 95%, or 99% sequence identity to SEQ ID NO: 37. Further provided herein are compositions, wherein the human IgE signal peptide comprises an amino acid sequence as set forth in SEQ ID NO: 37. Further provided herein are compositions, wherein the TGFbv2 comprises an amino acid sequence having at least 85%, 90%, 95%, or 99% sequence identity to SEQ ID NO: 8. Further provided herein are compositions, wherein the TGFbv2 comprises a sequence as set forth in SEQ ID NO: 8. Further provided herein are compositions, wherein the sequence encoding for the cytokine comprises a second promoter region that provides for expression of the cytokine, and wherein the sequence encoding for the TGF-beta activity inhibitor comprises a third promoter region that provides for expression of the TGF-beta activity inhibitor. Further provided herein are compositions, wherein each of the second and third promoter regions comprise a late promoter. Further provided herein are compositions, wherein the late promoter comprises any one of SSP, P7.5, P28, P135, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof. Further provided herein are compositions, wherein the late promoter comprises a weak late promoter. Further provided herein are compositions, wherein the weak late promoter comprises a P135 promoter. Further provided herein are compositions, wherein the second promoter region a P135 promoter. Further provided herein are compositions, wherein the late promoter comprises a strong late promoter. Further provided herein are compositions, wherein the strong late promoter comprises the P7.5 promoter. Further provided herein are compositions, wherein the third promoter region that provides for expression of the TGF-beta activity inhibitor comprises the P7.5 promoter. Further provided herein are compositions, wherein the exogenous nucleic acid encoding for a cytokine or a functional variant thereof, the exogenous nucleic acid encoding for the TGF-beta activity inhibitor, and the exogenous nucleic acid encoding for the chemokine receptor or functional variant thereof are located on a single genome. Further provided herein are compositions, wherein the vector is an oncolytic virus, and wherein the oncolytic virus comprises at least one genome modification. Further provided herein are compositions, wherein the at least one modification comprises mutation or deletion of at least one gene selected from the group consisting of: Thymidine Kinase (TK), F13L, A36R, A34R, A33R, A52R, B5R, B8R, B18R, SPI-1, SPI-2, B15R, VGF, E3L, K3L, A41L, K7R, or N1L, a functional fragment thereof, or any combination thereof. Further provided herein are compositions, wherein the at least one modification comprises a deletion of genes A52R and TK. Further provided herein are compositions, wherein the oncolytic virus is a poxvirus, an adeno associated virus, an adenovirus, a reovirus, a lentivirus, a herpes simplex virus, a vesicular stomatitis virus, a mengovirus, a myxoma virus, Newcastle disease virus, measles virus, or polio virus. Further provided herein are compositions, wherein the poxvirus is a vaccinia virus. Further provided herein are compositions, wherein the vaccinia virus is a Western Reserve strain. Further provided herein are compositions, wherein the at least one genome modification results in about 2, about 3, about 4, about 5, about 6, about 7, about 8, about 9, about 10, about 12, about 14, about 16, about 18, about 20, about 25, about 30, about 35, about 40, about 45, about 50, about 55, about 60, about 65, about 70, about 75, about 80, about 85, about 90, about 95, or about 100 fold increase in efficacy of tumor-targeted systemic delivery of the virus.

Provided herein are nucleic acids, wherein the nucleic acid comprises a sequence encoding for at least two polypeptides, wherein the at least two polypeptides comprise: interleukin-12 (IL-12) or a functional variant thereof; and a Transforming growth factor beta (TGF-beta) activity inhibitor. Provided herein are nucleic acids, wherein the nucleic acid comprises a sequence encoding for: a first polypeptide comprising an interleukin-12 (IL-12) or a functional variant thereof; and a second polypeptide comprising a Transforming growth factor beta (TGF-beta) activity inhibitor. Further provided herein are nucleic acids, wherein the nucleic acid comprises DNA or RNA. Further provided herein are nucleic acids, wherein the IL-12 is a murine IL-12 or a human IL-12. Further provided herein are nucleic acids, wherein the IL-12 or functional variant thereof comprises an alpha subunit and a beta subunit. Further provided herein are nucleic acids, wherein the alpha subunit and the beta subunit are connected by a linker. Further provided herein are nucleic acids, wherein the first polypeptide comprising the IL-12 alpha subunit and the IL-12 beta subunit further comprises a linker. Further provided herein are nucleic acids, wherein the IL-12 alpha subunit comprises a sequence having at least 85%, 90%, 95%, or 99% sequence identity to SEQ ID NO: 13 or SEQ ID NO: 16. Further provided herein are nucleic acids, wherein the IL-12 alpha subunit comprises a sequence as set forth in SEQ ID NO: 13 or SEQ ID NO: 16. Further provided herein are nucleic acids, wherein the IL-12 beta subunit comprises a sequence having at least 85%, 90%, 95%, or 99% sequence identity to SEQ ID NO: 14 or SEQ ID NO: 17. Further provided herein are nucleic acids, wherein the IL-12 beta subunit comprises a sequence as set forth in SEQ ID NO: 14 or SEQ ID NO: 17. Further provided herein are nucleic acids, wherein the linker comprises a sequence having at least 85%, 90%, 95%, or 99% sequence identity to SEQ ID NO: 18. Further provided herein are nucleic acids, wherein the linker comprises a sequence as set forth in SEQ ID NO: 18. Further provided herein are nucleic acids, wherein the encoded IL-12 comprises a sequence having at least 85%, 90%, 95%, or 99% sequence identity to SEQ ID NO: 12 or SEQ ID NO: 15. Further provided herein are nucleic acids, wherein the encoded IL-12 comprises a sequence as set forth in SEQ ID NO: 12 or SEQ ID NO: 15. Further provided herein are nucleic acids, wherein the nucleic acid encoding for IL-12 comprises a sequence having at least 85%, 90%, 95%, or 99% sequence identity to SEQ ID NO: 64 or SEQ ID NO: 66. Further provided herein are nucleic acids, wherein the nucleic acid encoding for IL-12 comprises a sequence as set forth in SEQ ID NO: 64 or SEQ ID NO: 66. Further provided herein are nucleic acids, wherein the TGF-beta activity inhibitor comprises a TGF-beta dominant negative, a TGF-beta receptor dominant negative, a protein that binds TGF-beta, or a protein that binds to a TGF-beta receptor. Further provided herein are nucleic acids, wherein the TGF-beta activity inhibitor comprises the protein that binds TGF-beta, and wherein the protein that binds to TBF-beta is an antibody or functional variant thereof. Further provided herein are nucleic acids, wherein the TGF-beta activity inhibitor comprises the protein that binds to TGF-beta receptor, and wherein the protein that binds to TGF-beta receptor is an antibody or functional variant thereof. Further provided herein are nucleic acids, wherein the TGF-beta activity inhibitor comprises the protein that binds to TGF-beta receptor, and wherein the protein that binds to TGF-beta receptor is a protein comprising a domain of TGF-beta. Further provided herein are nucleic acids, wherein the protein comprising a domain of TGF-beta comprises a sequence as set forth in any one of SEQ ID NOs: 1-9. Further provided herein are nucleic acids, wherein the protein comprises at least 85%, 90%, 95%, or 99% sequence identity to SEQ ID NO: 7 or SEQ ID NO: 8. Further provided herein are nucleic acids, wherein the protein comprising a domain of TGF-beta comprises a sequence as set forth in SEQ ID NO: 7 or SEQ ID NO: 8. Further provided herein are nucleic acids, wherein the nucleic acid encoding for the TGF-beta activity inhibitor comprises at least 85%, 90%, 95%, or 99% sequence identity to SEQ ID NO: 67. Further provided herein are nucleic acids, wherein the nucleic acid encoding for the TGF-beta activity inhibitor comprises a sequence as set forth in SEQ ID NO: 67. Further provided herein are nucleic acids, wherein the protein that binds to TGF-beta receptor comprises a TGF-beta fusion 1 (TGFbf1) protein. Further provided herein are nucleic acids, wherein the TGFbf1 comprises a murine IL-2 signal peptide and a TGF-beta variant 1 (TGFbv1). Further provided herein are nucleic acids, wherein the murine IL-2 signal peptide comprises a sequence having at least 85%, 90%, 95%, or 99% sequence identity to SEQ ID NO: 36. Further provided herein are nucleic acids, wherein the murine IL-2 signal peptide comprises a sequence as set forth in SEQ ID NO: 36. Further provided herein are nucleic acids, wherein the TGFbv1 comprises a sequence having at least 85%, 90%, 95%, or 99% sequence identity to SEQ ID NO: 7. Further provided herein are nucleic acids, wherein the TGFbv1 comprises a sequence as set forth in SEQ ID NO: 7. Further provided herein are nucleic acids, wherein the protein that binds to TGF-beta receptor comprises a TGF-beta fusion 2 (TGFbf2) protein. Further provided herein are nucleic acids, wherein the TGFbf2 comprises a human IgE signal peptide and a TGF-beta variant 2 (TGFbv2). Further provided herein are nucleic acids, wherein the human IgE signal peptide comprises a sequence having at least 85%, 90%, 95%, or 99% sequence identity to SEQ ID NO: 37. Further provided herein are nucleic acids, wherein the human IgE signal peptide comprises a sequence as set forth in SEQ ID NO: 37. Further provided herein are nucleic acids, wherein the TGFbv2 comprises a sequence having at least 85%, 90%, 95%, or 99% sequence identity to SEQ ID NO: 8. Further provided herein are nucleic acids, wherein the TGFbv2 comprises a sequence as set forth in SEQ ID NO: 8. Further provided herein are nucleic acids, wherein the protein that binds to the TGF-beta receptor binds to a TGF-beta receptor II and does not bind to a TGF-beta receptor I. Further provided herein are nucleic acids, further comprising at least one promoter region. Further provided herein are nucleic acids, wherein the at least one promoter region drives expression of the at least two polypeptides. Further provided herein are nucleic acids, wherein the at least one promoter region comprises a first promoter region and a second promoter region, wherein the first promoter region drives expression of the polypeptide comprising the IL-12 and the second promoter region drives expression of the TGF-beta activity inhibitor. Further provided herein are nucleic acids, wherein the first promoter region and the second promoter region each comprises any one of SSP, P7.5, P28, P135, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof. Further provided herein are nucleic acids, wherein the first promoter region comprises the P7.5 promoter. Further provided herein are nucleic acids, wherein the first promoter region comprises the P135 promoter. Further provided herein are nucleic acids, wherein the second promoter region comprises the P28 promoter. Further provided herein are nucleic acids, wherein the second promoter region comprises the P7.5 promoter. Further provided herein are nucleic acids, further comprising a sequence encoding for a chemokine receptor or a functional variant thereof. Further provided herein are nucleic acids, wherein the chemokine receptor comprises at least one of: a CXC receptor, a CC receptor, a CX3C receptor, an XC receptor, a functional fragment thereof, a functional variant thereof, or any combinations thereof. Further provided herein are nucleic acids, wherein the chemokine receptor comprises at least one of: CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, a functional fragment thereof or a functional variant thereof, or any combinations thereof. Further provided herein are nucleic acids, wherein the chemokine receptor is CXCR3. Further provided herein are nucleic acids, wherein the chemokine receptor comprises at least 85%, 90%, 95%, or 99% sequence identity to SEQ ID NO: 42 or SEQ ID NO: 43. Further provided herein are nucleic acids, wherein the chemokine receptor comprises a sequence as set forth in SEQ ID NO: 42 or SEQ ID NO: 43. Further provided herein are nucleic acids, wherein the sequence encoding the chemokine receptor further comprises a third promoter region that provides for expression of the chemokine receptor prior to expression of the IL-12 and the TGF-beta activity inhibitor. Further provided herein are nucleic acids, wherein the third promoter region comprises any one of A52R, pB8, mH5, I4L, LEO, pF11, I3L, P7.5, TK promoter, F7L, H5R, a short synthetic promoter (SSP) or any variation or combination thereof. Further provided herein are nucleic acids, wherein the third promoter comprises the A52R promoter.

Provided herein are nucleic acids, wherein the nucleic acid comprises: a first region encoding a first polypeptide comprising a sequence having at least 85%, 90%, 95%, or 99% sequence identity to SEQ ID NO: 12 or SEQ ID NO: 15; and a second region encoding a second polypeptide comprising a sequence having at least 85%, 90%, 95%, or 99% sequence identity to SEQ ID NO: 40 or SEQ ID NO: 41. Further provided herein are nucleic acids, wherein the nucleic acid comprises DNA or RNA. Further provided herein are nucleic acids, further comprising at least one promoter region. Further provided herein are nucleic acids, wherein the at least one promoter region drives expression of at least two polypeptides. Further provided herein are nucleic acids, comprising a first promoter region and a second promoter region, wherein the first promoter region drives expression of the first polypeptide and the second promoter region drives expression of the second polypeptide. Further provided herein are nucleic acids, wherein the first promoter region and the second promoter region each comprises any one of SSP, P7.5, P28, P135, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof. Further provided herein are nucleic acids, wherein the first promoter region comprises the P7.5 promoter. Further provided herein are nucleic acids, wherein the first promoter region comprise the P135 promoter. Further provided herein are nucleic acids, wherein the second promoter region comprises the P28 promoter. Further provided herein are nucleic acids, wherein the second promoter region comprises the P7.5 promoter. Further provided herein are nucleic acids, wherein: the first region encodes a polypeptide comprising the sequence as set forth in SEQ ID NO: 12 or SEQ ID NO: 15; and the second region encodes a polypeptide comprising the sequence as set forth in SEQ ID NO: 40 or SEQ ID NO: 41. Further provided herein are nucleic acids, wherein the nucleic acid comprises: a first region having at least 85%, 90%, 95%, or 99% sequence identity to SEQ ID NO: 64 or SEQ ID NO: 66; and a second region having at least 85%, 90%, 95%, or 99% sequence identity to SEQ ID NO: 65 or SEQ ID NO: 67. Further provided herein are nucleic acids, wherein the nucleic acid comprises a DNA and comprises a sequence in 5′ to 3′ order: a first region encoding for the IL-12 or functional variant thereof, and a second region encoding for the TGF-beta activity inhibitor. Further provided herein are nucleic acids, wherein the nucleic acid comprises a DNA encoding for sequences, in 5′ to 3′ order, as set forth in SEQ ID NO: 14, SEQ ID NO: 18, SEQ ID NO: 13, SEQ ID NO: 36, and SEQ ID NO: 7. Further provided herein are nucleic acids, wherein the nucleic acid comprises a DNA encoding for sequences in 5′ to 3′ order encoding for SEQ ID NO: 17, SEQ ID NO: 18, SEQ ID NO: 16, SEQ ID NO: 37, and SEQ ID NO: 8. Further provided herein are nucleic acids, wherein the nucleic acid comprises sequences encoding, in 5′ to 3′ order, for SEQ ID NO: 12, and SEQ ID NO: 40. Further provided herein are nucleic acids, wherein the nucleic acid comprises sequences encoding, in 5′ to 3′ order, for SEQ ID NO: 15, and SEQ ID NO: 41. Further provided herein are nucleic acids, further comprising a third region encoding a third polypeptide comprising a sequence having at least 85%, 90%, 95%, or 99% sequence identity to SEQ ID NO: 42 or SEQ ID NO: 43. Further provided herein are nucleic acids, wherein the third region comprises SEQ ID: 42 or SEQ ID NO: 43. Further provided herein are nucleic acids, further comprising a third region having at least 85%, 90%, 95%, or 99% sequence identity to SEQ ID NO: 69 or SEQ ID NO: 86. Further provided herein are nucleic acids, wherein the third region comprises a sequence set forth in SEQ ID NO: 69 or SEQ ID NO: 86. Further provided herein are nucleic acids, wherein the third region encoding the third polypeptide further comprises a third promoter region that provides for expression of the third polypeptide prior to expression of the first and second polypeptides. Further provided herein are nucleic acids, wherein the third promoter comprises any one of A52R, pB8, mH5, I4L, LEO, pF11, I3L, P7.5, TK promoter, F7L, H5R, a short synthetic promoter (SSP) or any variation or combination thereof. Further provided herein are nucleic acids, wherein the third promoter comprises the A52R promoter. Further provided herein are nucleic acids, wherein the nucleic acid is present in an oncolytic virus. Further provided herein are nucleic acids, wherein the oncolytic virus is a poxvirus, an adeno associated virus, an adenovirus, a reovirus, a lentivirus, a herpes simplex virus, a vesicular stomatitis virus, a mengovirus, a myxoma virus, Newcastle disease virus, measles virus, or polio virus. Further provided herein are nucleic acids, wherein the poxvirus is a vaccinia virus. Further provided herein are nucleic acids, wherein the vaccinia virus is a modified strain of Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Further provided herein are nucleic acids, wherein the nucleic acid is inserted into the viral genome. Further provided herein are nucleic acids, further comprising mutation or deletion of at least one viral gene selected from the group consisting of: Thymidine Kinase (TK), F13L, A36R, A34R, A33R, A52R, B5R, B8R, B18R, SPI-1, SPI-2, B15R, VGF, E3L, K3L, A41L, K7R, or N1L, a functional fragment thereof, or any combinations thereof.

Provided herein are nucleic acid molecules, wherein the nucleic acid molecule comprises: an A52R locus comprising, in 5′ to 3′ order: a first promoter region, wherein the promoter comprises an A52R promoter; an insertion of a first region encoding human CXCR3; an insertion at a TK gene locus comprising, in 5′ to 3′ order: a second promoter region, wherein the promoter comprises P135; a second region encoding human IL-12; a third promoter region, wherein the promoter comprises P7.5; and a third region encoding a TGF beta variant. Further provided herein are nucleic acid molecules, wherein the P135 promoter comprises a sequence as set forth in SEQ ID NO: 56 and the P7.5 promoter comprises a sequence as set forth in SEQ ID NO: 57. Further provided herein are nucleic acid molecules, wherein the first region encoding human CXCR3 comprises a sequence as set forth in SEQ ID NO: 86; wherein the second region encoding human IL-12 comprises a sequence as set forth in SEQ ID NO: 66; and wherein the third region encoding a TGF-beta inhibitor comprises a sequence as set forth in SEQ ID NO: 67. Further provided herein are nucleic acid molecules, wherein the nucleic acid molecule comprises: an insertion at an A52R locus comprising a sequence as set forth in SEQ ID NO: 88; an insertion at a TK gene locus comprising a sequence as set forth in SEQ ID NO: 85.

Provided herein are pharmaceutical compositions, wherein the pharmaceutical composition comprises: a nucleic acid as described herein or a vector as described herein; and a pharmaceutically acceptable excipient. Further provided herein are pharmaceutical compositions, wherein the composition is in a liquid dosage form. Further provided herein are pharmaceutical compositions, wherein the pharmaceutically acceptable excipient is a buffered saline. Further provided herein are pharmaceutical compositions, wherein the buffered saline is phosphate buffered saline (PBS), Dulbecco's PBS (DPBS), TRIS-buffered saline (TBS), Hank's balanced salt solution (HBSS), Earle's balanced salt solution (EBSS), standard saline citrate (SSC), HEPES-buffered saline (HBS), or Gey's balanced salt solution. Further provided herein are pharmaceutical compositions, wherein the composition further comprises a liposome or nanoparticle. Further provided herein are pharmaceutical compositions, wherein the nucleic acid or vector is associated with the liposome or nanoparticle.

Provided herein are methods for treatment of cancer comprising administering to a subject having cancer a pharmaceutical composition as described herein in an amount sufficient for treatment of a cancer. Further provided herein are methods, wherein the cancer is a solid tumor or a blood cancer. Further provided herein are methods, wherein the cancer comprises melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods, wherein the administering comprises an intratumoral administration. Further provided herein are methods, wherein the administering comprises a systemic administration. Further provided herein are methods, wherein the systemic administration comprises oral administration, parenteral administration, intranasal administration, sublingual administration, rectal administration, transdermal administration, or any combinations thereof.

Provided herein are methods for activating an anti-tumor immune response, comprising administering to a subject having a cancer a pharmaceutical composition as described herein. Further provided herein are methods, wherein the cancer is a solid tumor, a leukemia, or a lymphoma. Further provided herein are methods, wherein the cancer comprises melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods, wherein the administering step is an intratumoral administration. Further provided herein are methods, wherein the administering step is a systemic administration. Further provided herein are methods, wherein the systemic administration comprises oral administration, parenteral administration, intranasal administration, sublingual administration, rectal administration, transdermal administration, or any combinations thereof.

Provided herein are methods for reduction of incidence of tumor cell growth, comprising: administering to tumor cells a pharmaceutical composition as described herein in an effective amount sufficient for reduction of incidence of tumor cell growth. Further provided herein are methods, wherein the tumor cells are from a solid tumor or a blood cancer. Further provided herein are methods, wherein the tumor cells are from a melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods, wherein the administering step is an intratumoral administration.

### EXAMPLES

The examples below further illustrate the described embodiments without limiting the scope of this disclosure.

### Example 1: mIL12 and TGFbf1 Construct Design

A vaccinia virus comprising the construct depicted in FIG. 2 was constructed by substituting the open reading frame encoding for a viral thymidine kinase with nucleic acids comprising sequences encoding a murine IL-12 (murine IL-12) (SEQ ID NO: 64) and a TGF-beta fusion (TGF-bf1) (SEQ ID NO: 65) as shown in Table 9. Expression of the murine IL-12 was driven by viral promoter P7.5 (SEQ ID NO: 57). Expression of the TGF-bf1 was driven by viral promoter P28 (SEQ ID NO: 58). The murine IL-12 polypeptide comprised a covalent dimer consisting of the murine interleukin-12 subunit beta (mIL-12b) (UniProtKB accession ID P43432. I) (SEQ ID NO: 14) linked by a 22-residue glycine-rich linker (SEQ ID NO: 18) to murine interleukin-12 subunit alpha (mIL-12a) (UniProtKB accession ID 43431) residues 11-215 (SEQ ID NO: 13). The TGFbf1 polypeptide comprised the signal peptide of murine Interleukin-2 (IL-2sig) (UniProtKB accession ID P04351.1) (SEQ ID NO: 36) fused to a TGF-beta inhibitor with cysteines 8 and 17 (original PDB numbering) mutated to valine and alanine, respectively (TGFbv1) (SEQ ID NO: 7).

TABLE 9
Nucleic acid sequences.

| SEQ ID | | |
|---|---|---|
| NO: | Description | Nucleic Acid Sequence |
| 64 | murine IL- | ATGTGTCCTCAGAAGCTAACCATCTCCTGGTTTGCCATCGTTTTGCT |
| | 12 | GGTGTCTCCACTCATGGCCATGTGGGAGCTGGAGAAAGACGTTTA |
| | | TGTTGTAGAGGTGGACTGGACTCCCGATGCCCCTGGAGAAACAGT |
| | | GAACCTCACCTGTGACACGCCTGAAGAAGATGACATCACCTGGAC |
| | | CTCAGACCAGAGACATGGAGTCATAGGCTCTGGAAAGACCCTGAC |
| | | CATCACTGTCAAAGAGTTTCTAGATGCTGGCCAGTACACCTGCCAC |
| | | AAAGGAGGCGAGACTCTGAGCCACTCACATCTGCTGCTCCACAAG |
| | | AAGGAAAATGGAATTTGGTCCACTGAAATTTTAAAAAATTTCAAA |
| | | AACAAGACTTTCCTGAAGTGTGAAGCACCAAATTACTCCGGACGG |
| | | TTCACGTGCTCATGGCTGGTGCAAAGAAACATGGACTTGAAGTTC |
| | | AACATCAAGAGCAGTAGCAGTTCCCCTGACTCTCGGGCAGTGACA |
| | | TGTGGAATGGCGTCTCTGTCTGCAGAGAAGGTCACACTGGACCAA |
| | | AGGGACTATGAGAAGTATTCAGTGTCCTGCCAGGAGGATGTCACC |
| | | TGCCCAACTGCCGAGGAGACCCTGCCCATTGAACTGGCGTTGGAA |
| | | GCACGGCAGCAGAATAAATATGAGAACTACAGCACCAGCTTCTTC |
| | | ATCAGGGACATCATCAAACCAGACCCGCCCAAGAACTTGCAGATG |
| | | AAGCCTTTGAAGAACTCACAGGTGGAGGTCAGCTGGGAGTACCCT |
| | | GACTCCTGGAGCACTCCCCATTCCTACTTCTCCCTCAAGTTCTTTGT |
| | | TCGAATCCAGCGCAAGAAAGAAAAGATGAAGGAGACAGAGGAGG |
| | | GGTGTAACCAGAAAGGTGCGTTCCTCGTAGAGAAGACATCTACCG |
| | | AAGTCCAATGCAAAGGCGGGAATGTCTGCGTGCAAGCTCAGGATC |
| | | GCTATTACAATTCCTCATGCAGCAAGTGGGCATGTGTTCCATGCAG |
| | | GGTCCGATCCGGAGGTGGCGGATCCGGCGGAGGTGGCTCAGGTGG |
| | | CGGTGGATCAGGAGGAGGTGGATCAGCTAGCGCCACCCTTGCCCT |
| | | CCTAAACCACCTCAGTTTGGCCAGGGTCATTCCAGTCTCTGGACCT |
| | | GCCAGGTGTCTTAGCCAGTCCCGAAACCTGCTGAAGACCACAGAT |
| | | GACATGGTGAAGACGGCCAGAGAAAAACTGAAACATTATTCCTGC |
| | | ACTGCTGAAGACATCGACCATGAAGACATCACACGGGACCAAACC |
| | | AGCACATTGAAGACCTGTTTACCACTGGAACTACACAAGAACGAG |
| | | AGTTGCCTGGCTACTAGAGAGACTTCTTCCACAACAAGAGGGAGC |
| | | TGCCTGCCCCCACAGAAGACGTCTTTGATGATGACCCTGTGCCTTG |
| | | GTAGCATCTATGAGGACTTGAAGATGTACCAGACAGAGTTCCAGG |
| | | CCATCAACGCAGCACTTCAGAATCACAACCATCAGCAGATCATTCT |
| | | AGACAAGGGCATGCTGGTGGCCATCGACGAGCTGATGCAGTCTCT |
| | | GAATCATAATGGCGAGACTCTGCGCCAGAAACCTCCTGTGGGAGA |
| | | AGCAGACCCTTACAGAGTGAAAATGAAGCTCTGCATCCTGCTTCA |
| | | CGCCTTCAGCACCCGCGTCGTGACCATCAACAGGGTGATGGGCTA |
| | | TCTGAGTTCCGCCTAA |
| 65 | TGF-bf1 | ATGTATTCTATGCAACTGGCATCATGCGTTACCCTTACCTTGGTGC |
| | | TTCTCGTCAATAGCGCTCTGGACGCTGCTTACGTCTTCCGTAACGT |
| | | TCAGGACAACTGCGCTCTGCGTCCGCTGTACATCGACTTCCGCAAA |
| | | GACCTGGGTTGGAAATGGATCCACGAACCGAAAGGTTACAACGCT |
| | | AACTTCTGCGCTGGTGCTTGCCCGTACCGTGCTTCCAAATCCCCGC |
| | | GCTGCCGATCCCAGGACCTGGAACCGCTGACCATCGTGTACTACGT |
| | | GGGTCGCAAACCGAAAGTGGAACAGCTGTCCAACATGATCGTTAA |
| | | ATCCTGCAAATGCTCCTAG |

### Example 2: hIL12 and TGFbf2 Construct Design

A vaccinia virus comprising the construct depicted in FIG. 2 was constructed by substituting the open reading frame encoding for a viral thymidine kinase with nucleic acids comprising sequences encoding a human IL-12 (SEQ ID NO: 66) and a TGF-beta fusion (TGF-bf1) (SEQ ID NO: 67) as shown in Table 10. Expression of the hIL-12 was driven by viral promoter P135 (SEQ ID NO: 57). Expression of the TGF-bf2 was driven by viral promoter P7.5 (SEQ ID NO: 57). The human IL-12 polypeptide comprises a covalent dimer consisting of the human interleukin-12 subunit beta (hIL-12b) (SEQ ID NO: 17) linked by a 22-residue glycine-rich linker (SEQ ID NO: 18) to human interleukin-12 subunit alpha (hIL-12a) (SEQ ID NO: 16). The TGFbf2 polypeptide comprises the signal peptide of human IgE (SEQ ID NO: 37) fused to a TGF-beta inhibitor comprising the cystine-knot region of PRDC (TGFbv2) (SEQ ID NO: 8).

TABLE 10
Nucleic acid sequences.

| SEQ ID | | |
|---|---|---|
| NO: | Description | Nucleic Acid Sequence |
| 66 | human IL- | ATGTGTCACCAGCAGTTGGTCATCTCTTGGTTTTCCCTGGTTTTTCTG |
| | 12 | GCATCTCCCCTCGTGGCCATATGGGAACTGAAGAAAGATGTTTATG |
| | | TCGTAGAATTGGATTGGTATCCGGATGCCCCTGGAGAAATGGTGGT |
| | | CCTCACCTGTGACACCCCTGAAGAAGATGGTATCACCTGGACCTTG |
| | | GACCAGAGCAGTGAGGTCTTAGGCTCTGGCAAAACCCTGACCATCC |
| | | AAGTCAAAGAGTTTGGAGATGCTGGCCAGTACACCTGTCACAAAGG |
| | | AGGCGAGGTTCTAAGCCATTCGCTCCTGCTGCTTCACAAAAAGGAA |
| | | GATGGAATTTGGTCCACTGATATTTTAAAGGACCAGAAAGAACCCA |
| | | AAAATAAGACCTTTCTAAGATGCGAGGCCAAGAATTATTCTGGACG |
| | | TTTCACCTGCTGGTGGCTGACGACAATCAGTACTGATTTGACATTCA |
| | | GTGTCAAAAGCAGCAGAGGCTCTTCTGACCCCCAAGGGGTGACGTG |
| | | CGGAGCTGCTACACTCTCTGCAGAGAGAGTCAGAGGGGACAACAA |
| | | GGAGTATGAGTACTCAGTGGAGTGCCAGGAGGACAGTGCCTGCCC |
| | | AGCTGCTGAGGAGAGTCTGCCCATTGAGGTCATGGTGGATGCCGTT |
| | | CACAAGCTCAAGTATGAAAACTACACCAGCAGCTTCTTCATCAGGG |
| | | ACATCATCAAACCTGACCCACCCAAGAACTTGCAGCTGAAGCCATT |
| | | AAAGAATTCTCGGCAGGTGGAGGTCAGCTGGGAGTACCCTGACACC |
| | | TGGAGTACTCCACATTCCTACTTCTCCCTGACATTCTGCGTTCAGGT |
| | | CCAGGGCAAGAGCAAGAGAGAAAAGAAAGATAGAGTCTTCACGGA |
| | | CAAGACCTCAGCCACGGTCATCTGCCGCAAAAATGCCAGCATTAGC |
| | | GTGCGGGCCCAGGACCGCTACTATAGCTCATCTTGGAGCGAATGGG |
| | | CATCTGTGCCCTGCAGTGGAGGTGGCGGATCCGGCGGAGGTGGCTC |
| | | AGGTGGCGGTGGATCAGGAGGAGGTGGATCAGCTAGCTGGCCCCC |
| | | TGGGTCAGCCTCCCAGCCACCGCCCTCACCTGCCGCGGCCACAGGT |
| | | CTGCATCCAGCGGCTCGCCCTGTGTCCCTGCAGTGCCGGCTCAGCA |
| | | TGTGTCCAGCGCGCAGCCTCCTCCTTGTGGCTACCCTGGTCCTCCTG |
| | | GACCACCTCAGTTTGGCCAGAAACCTCCCCGTGGCCACTCCAGACC |
| | | CAGGAATGTTCCCATGCCTTCACCACTCCCAAAACCTGCTGAGGGC |
| | | CGTCAGCAACATGCTCCAGAAGGCCAGACAAACTCTAGAATTTTAC |
| | | CCTTGCACTTCTGAAGAGATTGATCATGAAGATATCACAAAAGATA |
| | | AAACCAGCACAGTGGAGGCCTGTTTACCATTGGAATTAACCAAGAA |
| | | TGAGAGTTGCCTAAATTCCAGAGAGACCTCTTTCATAACTAATGGG |
| | | AGTTGCCTGGCCTCCAGAAAGACCTCTTTTATGATGGCCCTGTGCCT |
| | | TAGTAGTATTTATGAAGACTTGAAGATGTACCAGGTGGAGTTCAAG |
| | | ACCATGAATGCAAAGCTTCTGATGGATCCTAAGAGGCAGATCTTTC |
| | | TAGATCAAAACATGCTGGCAGTTATTGATGAGCTGATGCAGGCCCT |
| | | GAATTTCAACAGTGAGACTGTGCCACAAAAATCCTCCCTTGAAGAA |
| | | CCGGATTTTTATAAAACTAAAATCAAGCTCTGCATACTTCTTCATGC |
| | | TTTCAGAATTCGGGCAGTGACTATTGATAGAGTGATGAGCTATCTG |
| | | AATGCTTCCTAA |
| 67 | TGF-bf2 | ATGGACTGGACATGGATTCTCTTTCTAGTGGCCGCAGCCACAAGGG |
| | | TCCACAGCGGCCGCAAGGAAGTCCTTGCTTCTAGTCAGGAAGCGCT |
| | | CGTTGTAACAGAAAGAAAATATCTCAAATCCGACTGGTGTAAGCTG |
| | | CGACCACTCTACATAGATTTTCGAAAGGACCTGGGCTGGAAATGGA |
| | | TACATGAGCCAAAGGGCTATAATGCGAATTTTTGCTACGGACAATG |
| | | TAATTCTTTCTATATCCCCCGGCACGTCAAAAAGGAGGAAGATTCT |
| | | TTCCAATCATCTGCATTCTGTGTAAGCCAGGATCTCGAGCCGTTGAC |
| | | TATTGTCTACTACGTTGGAAGAAAACCGAAGGTCGAGCAACTGTCT |
| | | AACATGATCGTCAAATCATGCAGGTGCATGTCAGTGTGA |

### Example 3: Measurement of Tumor Growth after Treatment of TGF-beta Inhibitor and IL-12

Renca cells were implanted into the flank of Balb/c mice. After 12 days mice were sorted such that each treatment group had an average of 58 mm³. Mice were treated with 1E7 PFU virus via IT injection.

B16F10 cells were mixed 1:1 with Matrigel and implanted into the flank of C57/Black 6 mice. After 5 days mice were sorted such that each treatment group had an average of 93 mm³. Mice were treated with 1E7 PFU virus via IT injection.

Tumors were measured 2× per week, and mice were weighed 1× per week. Mice were monitored for signs of morbidity and mortality, none were observed (Data not shown).

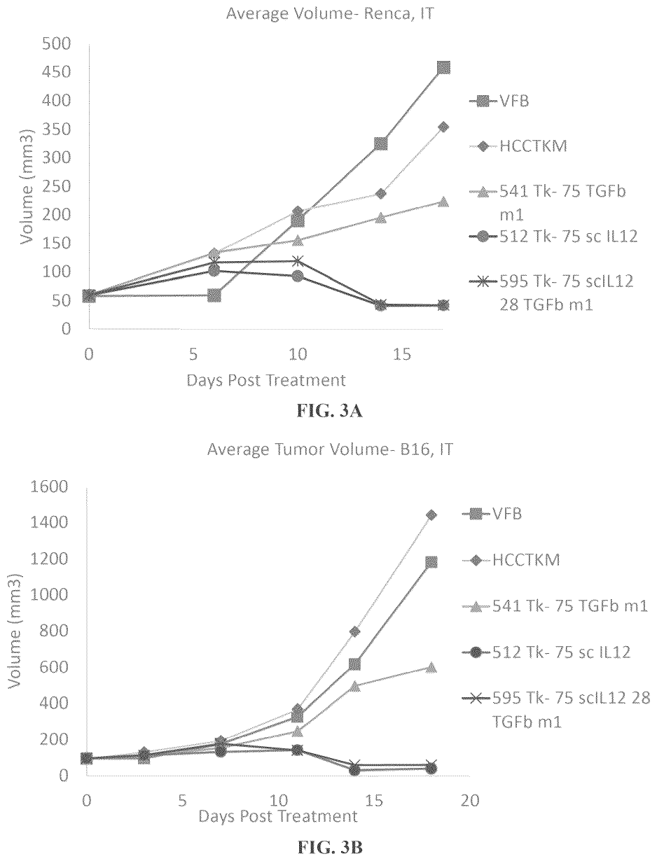

Mice induced with tumors were divided into treatment groups including PBS, TK-, TK-expressing IL12, TK-expressing TGF-beta inhibitor, and TK-expressing IL-12 and TGF-beta inhibitor. Average tumor volume was measured in each treatment group, as shown in FIG. 3A, for Renca tumor groups, and FIG. 3B, for B16 tumor groups.

Mice with induced Renca tumors treated with virus expressing TGF-beta inhibitor alone showed an average tumor volume of about 225 mm³ 17 days post treatment. Groups treated with virus expressing the combination IL-12 and TGF-beta inhibitor or IL-12 alone showed an average tumor volume of less than 50 mm³ 17 days post treatment. In comparison, control mice treated with PBS had an average tumor volume of about 450 mm³.

Mice with induced B16 tumors treated with virus expressing TGF-beta inhibitor alone had an average tumor volume of about 600 mm³ 17 days post treatment. Mice treated with virus expressing the combination IL-12 and TGF-beta inhibitor or IL-12 alone had an average tumor volume of less than 100 mm³ 17 days post treatment. In comparison, control mice treated with PBS had an average tumor volume of about 1200 mm³.

Treatment with a modified virus expressing both TGF-beta inhibitor and IL-12 inhibited tumor growth in mice more than treatment with virus expressing TGF-beta inhibitor alone.

### Example 4: Survival Assay with TGF-Beta Inhibitor and mIL-12

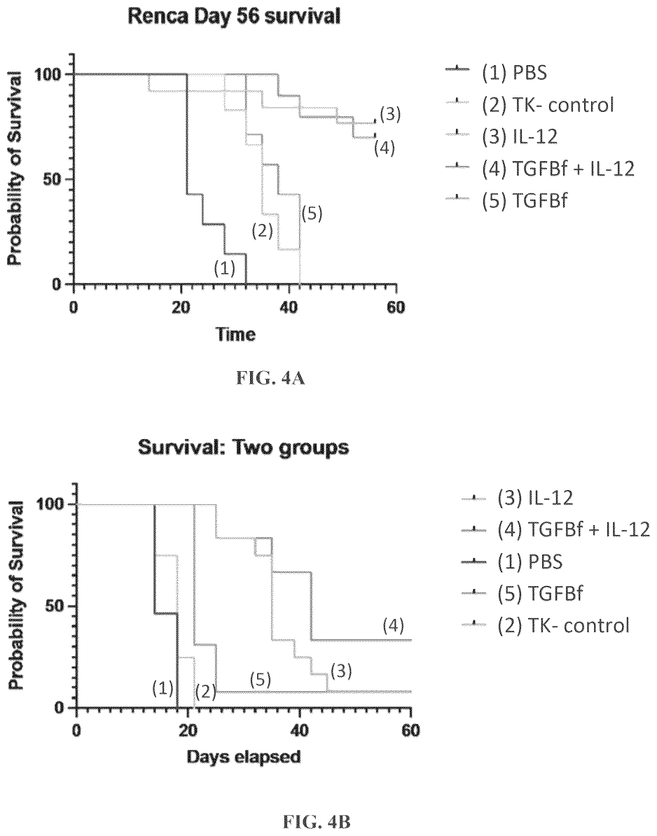

Mice were induced with tumors using Renca and B16 cells, then treated with modified viruses as described in Example 2. Treatment groups included PBS, TK-virus, TK-virus expressing IL12, TK-virus expressing TGF-beta inhibitor, and TK-virus expressing IL-12 and TGF-beta inhibitor. Probability of survival was calculated in each treatment group, as shown in FIG. 4A, for Renca tumor groups, and FIG. 4B, for B16 tumor groups. In mice with Renca cell tumors, treatment groups receiving virus expressing IL-12 or IL-12 and TGF-beta inhibitor showed about 75% survival after 56 days, while the other groups had zero surviving subjects by Day 56. In mice with B16 tumors, those treated with modified virus expressing TGF-beta inhibitor and IL-12 showed about 35% survival, compared to 0 surviving subjects in other treatment groups.

### Example 5: Addition of Murine CXCR3 Expression System

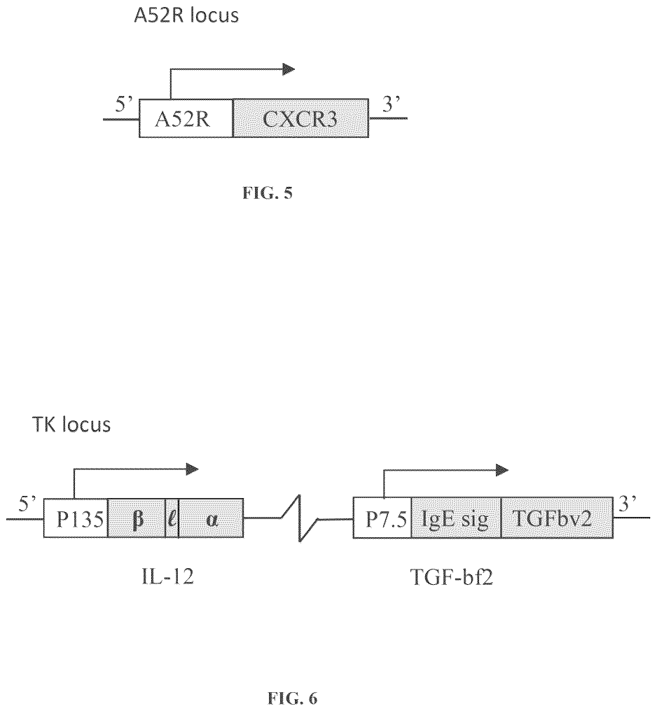

A vaccinia virus was modified by replacing the gene encoding A52 with nucleic acid encoding murine CXCR3 and a fluorescent reporter. A plasmid transfer vector was generated comprising, in order, with no gaps, an upstream recombination directing sequence A (SEQ ID NO: 68), an open reading frame encoding murine CXCR3 (SEQ ID NO: 69), a stop codon, SacI cloning site, and short spacer (SEQ ID NO: 70), a loxP site (SEQ ID NO: 71), a spacer followed by viral promoter driving expression of GFP-pac reporter (SEQ ID NO: 72), PacI cloning site and short spacer A (SEQ ID NO: 73), a loxP site (SEQ ID NO: 71), and a downstream recombination directing sequence A (SEQ ID NO: 74). Following recombination and treatment with cre recombinase, the viral genome comprised the incorporated sequence as set forth in SEQ ID NO: 75. Sequences are set forth in Table 11. A schematic representation of the promoter and gene expression construct is shown in FIG. 5.

TABLE 11
Murine CXCR3 recombination sequences.

| SEQ | | |
|---|---|---|
| ID NO: | Description | Nucleic acid sequence |
| 68 | Upstream recombination | CTCATCAGACATATCGTGTGCATGCGTGGAGTTA |
| | directing sequence A | AATTCATATCACGTAGCCATTCTTCCCATGGATGT |
| | | TTCCTTTTTTACCAAAGGAAATGCATCATTGATTA |
| | | TTCTCCTGTTTGATTTCTCTATCGATGCAGCACCT |
| | | CTCTTAAGAAGTGTAACCGATAATAATGTTATTA |
| | | TATCTAGACACCAGCGTCTACATGACGAGCTTCC |
| | | GAGTTCCAATTGGTTCAAGTTTTACATAAGTATA |
| | | AAGTCCGACTATTGTTCTATATTATATATGGTTGT |
| | | TGATGGATCTGTGATGCATGCGATAGCTGATAAT |
| | | AGAACTCACGCAATTATTAGCAAAAATATATTAG |
| | | ACAATACTACGATTAACGATGAGTGTAGATGCTG |
| | | TTATTTTGAACCACAGATTAGGATTCTTGATAGA |
| | | GATGAGATGCTCAATGGATCATCGTGTGATATGA |
| | | ACAGACATTGTATTATGATGAATTTACCTGATGT |
| | | AGGCAAATTTGGATCTAGTATGTTGGGGAAATAT |
| | | GAACCTGACATGATTAAGATTGCTCTTTCGGTGG |
| | | CTGGTAATTTAATAAGAAATCGAGACTACATTCC |
| | | CGGGAGACGAGGATATAGCTACTACGTTTACGGT |
| | | ATAGCCTCTAGATAATTTTTTTAAGCACGAAATA |
| | | AAAAACATAATTTTAAACCAATCTATTTCATACT |
| | | ATTTTGTGTGATCACC |
| 69 | Open reading frame encoding | ATGTACCTTGAGGTTAGTGAACGTCAAGTGCTAG |
| | murine CXCR3 | ATGCCTCGGACTTTGCCTTTCTTCTGGAAAACAGC |
| | | ACCTCTCCCTACGATTATGGGGAAAACGAGAGCG |
| | | ACTTCTCTGACTCCCCGCCCTGCCCACAGGATTTC |
| | | AGCCTGAACTTTGACAGAACCTTCCTGCCAGCCC |
| | | TCTACAGCCTCCTCTTCTTGCTGGGGCTGCTAGGC |
| | | AATGGGGCGGTGGCTGCTGTGCTACTGAGTCAGC |
| | | GCACTGCCCTGAGCAGCACGGACACCTTCCTGCT |
| | | CCACCTGGCTGTAGCCGATGTTCTGCTGGTGTTAA |
| | | CTCTTCCATTGTGGGCAGTGGATGCTGCTGTCCAG |
| | | TGGGTTTTCGGCCCTGGCCTCTGCAAAGTGGCAG |
| | | GCGCCTTGTTCAACATCAACTTCTATGCAGGGGC |
| | | CTTCCTGCTGGCTTGTATAAGCTTCGACAGATATC |
| | | TGAGCATAGTGCACGCCACCCAGATCTACCGCAG |
| | | GGACCCCCGGGTACGTGTAGCCCTCACCTGCATA |
| | | GTTGTATGGGGTCTCTGTCTGCTCTTTGCCCTCCC |
| | | AGATTTCATCTACCTATCAGCCAACTACGATCAG |
| | | CGCCTCAATGCCACCCATTGCCAGTACAACTTCC |
| | | CACAGGTGGGTCGCACTGCTCTGCGTGTACTGCA |
| | | GCTAGTGGCTGGTTTCCTGCTGCCCCTTCTGGTCA |
| | | TGGCCTACTGCTATGCCCATATCCTAGCTGTTCTG |
| | | CTGGTCTCCAGAGGCCAGAGGCGTTTTCGAGCTA |
| | | TGAGGCTAGTGGTAGTGGTGGTGGCAGCCTTTGC |
| | | TGTCTGCTGGACCCCCTATCACCTGGTGGTGCTAG |
| | | TGGATATCCTCATGGATGTGGGAGTTTTGGCCCG |
| | | CAACTGTGGTCGAGAAAGCCACGTGGATGTGGCC |
| | | AAGTCAGTCACCTCGGGCATGGGGTACATGCACT |
| | | GCTGCCTCAATCCGCTGCTCTATGCCTTTGTGGGA |
| | | GTGAAGTTCAGAGAGCAAATGTGGATGTTGTTCA |
| | | CGCGCCTGGGCCGCTCTGACCAGAGAGGGCCCCA |
| | | GCGGCAGCCGTCATCTTCACGGAGAGAATCATCC |
| | | TGGTCTGAGACAACTGAGGCCTCCTACCTGGGCT |
| | | TGTAA |
| 70 | Stop codon, SacI cloning | TAGGAGCTCTAGA |
| | site, and short spacer | |
| 71 | loxP site | ATAACTTCGTATAGCATACATTATACGAAGTTAT |
| 72 | Spacer followed by viral | AATTCTGCAGATGGATCATAAAAATATAGTAGAA |
| | promoter driving expression | TTTCATTTTGTTTTTTTCTATGCTATAAATAGCTTC |
| | of GFP-pac reporter | TCGAGCATGCGTAAAGGCGAAGAGCTGTTCACTG |
| | | GTGTCGTCCCTATTCTGGTGGAACTGGATGGTGA |
| | | TGTCAACGGTCATAAGTTTTCCGTGCGTGGCGAG |
| | | GGTGAAGGTGACGCAACTAATGGTAAACTGACGC |
| | | TGAAGTTCATCTGTACTACTGGTAAACTGCCGGT |
| | | CCCTTGGCCGACTCTGGTAACGACGCTGACTTAT |
| | | GGTGTTCAGTGCTTTGCTCGTTATCCGGACCATAT |
| | | GAAGCAGCATGACTTCTTCAAGTCCGCCATGCCG |
| | | GAAGGCTATGTGCAGGAACGCACGATTTCCTTTA |
| | | AGGATGACGGCACGTACAAAACGCGTGCGGAAG |
| | | TGAAATTTGAAGGCGATACCCTGGTAAACCGCAT |
| | | TGAGCTGAAAGGCATTGACTTTAAAGAAGACGGC |
| | | AATATCCTGGGCCATAAGCTGGAATACAATTTTA |
| | | ACAGCCACAATGTTTACATCACCGCCGATAAACA |
| | | AAAAAATGGCATTAAAGCGAATTTTAAAATTCGC |
| | | CACAACGTGGAGGATGGCAGCGTGCAGCTGGCTG |
| | | ATCACTACCAGCAAAACACTCCAATCGGTGATGG |
| | | TCCTGTTCTGCTGCCAGACAATCACTATCTGAGCA |
| | | CGCAAAGCGTTCTGTCTAAAGATCCGAACGAGAA |
| | | ACGCGATCATATGGTTCTGCTGGAGTTCGTAACC |
| | | GCAGCGGGCATCACGCATGGTATGGATGAACTGT |
| | | ACAAAGGAGGCTCAGGAACCGAGTACAAGCCCA |
| | | CGGTGCGCCTCGCCACCCGCGACGACGTCCCCCG |
| | | GGCCGTACGCACCCTCGCCGCCGCGTTCGCCGAC |
| | | TACCCCGCCACGCGCCACACCGTCGACCCGGACC |
| | | GCCACATCGAGCGGGTCACCGAGCTGCAAGAACT |
| | | CTTCCTCACGCGCGTCGGGCTCGACATCGGCAAG |
| | | GTGTGGGTCGCGGACGACGGCGCCGCGGTGGCG |
| | | GTCTGGACCACGCCGGAGAGCGTCGAAGCGGGG |
| | | GCGGTGTTCGCCGAGATCGGCCCGCGCATGGCCG |
| | | AGTTGAGCGGTTCCCGGCTGGCCGCGCAGCAACA |
| | | GATGGAAGGCCTCCTGGCGCCGCACCGGCCCAAG |
| | | GAGCCCGCGTGGTTCCTGGCCACCGTCGGCGTCT |
| | | CGCCCGACCACCAGGGCAAGGGTCTGGGCAGCG |
| | | CCGTCGTGCTCCCCGGAGTGGAGGCGGCCGAGCG |
| | | CGCCGGGGTGCCCGCCTTCCTGGAGACCTCCGCG |
| | | CCCCGCAACCTCCCCTTCTACGAGCGGCTCGGCTT |
| | | CACCGTCACCGCCGACGTCGAGTGCCCGAAGGAC |
| | | CGCGCGACCTGGTGCATGACCCGCAAGCCCGGTG |
| | | CCTGA |
| 73 | PacI cloning site and short | TTAATTAAGGCCGCTCGA |
| | spacer A | |
| 74 | Downstream recombination | AAAGTTAAAAGTTAATGAGAGCAAAAATATATA |
| | directing sequence A | AGGTTGTATTCCATATTTGTTATTTTTTCTGTAAT |
| | | AGTTAGAAAAATACATTCGATGGTCTATCTACCA |
| | | GATTATTATGTGTTATAAGGTACTTTTTCTCATAA |
| | | TAAACTAGAGTATGAGTAAGATAGTGTTTTTCAA |
| | | AACATATAAATCTAAAATTGATGGATGAGATATA |
| | | CAGCTATTAATTTCGAAAATATATTTTAATCTGAT |
| | | AACTTTAAACATGGATTTTTGATGGTGGTTTAACG |
| | | TTTTAAAAAAAGATTTTGTTATTGTAGTATATGAT |
| | | AATATTAAAAGATGGATATAAAGAATTTGCTGAC |
| | | TGCATGTACTATTTTTTACATTACTACATTGGCTA |
| | | CGGCAGATATACCTACTCCGCCACCAACGGGTCA |
| | | TGTGACAAGGGAGAATATCTTGATAAGAGGCATA |
| | | ATCAATGTTGTAATCGGTGTCCACCTGGAGAATT |
| | | TGCCAAGGTTAGATGTAAT |
| 75 | Sequence incorporated in | CTCATCAGACATATCGTGTGCATGCGTGGAGTTA |
| | viral genome (non-native | AATTCATATCACGTAGCCATTCTTCCCATGGATGT |
| | DNA BOLD; CXCR3 transgene | TTCCTTTTTTACCAAAGGAAATGCATCATTGATTA |
| | UNDERLINED) | TTCTCCTGTTTGATTTCTCTATCGATGCAGCACCT |
| | | CTCTTAAGAAGTGTAACCGATAATAATGTTATTA |
| | | TATCTAGACACCAGCGTCTACATGACGAGCTTCC |
| | | GAGTTCCAATTGGTTCAAGTTTTACATAAGTATA |
| | | AAGTCCGACTATTGTTCTATATTATATATGGTTGT |
| | | TGATGGATCTGTGATGCATGCGATAGCTGATAAT |
| | | AGAACTCACGCAATTATTAGCAAAAATATATTAG |
| | | ACAATACTACGATTAACGATGAGTGTAGATGCTG |
| | | TTATTTTGAACCACAGATTAGGATTCTTGATAGA |
| | | GATGAGATGCTCAATGGATCATCGTGTGATATGA |
| | | ACAGACATTGTATTATGATGAATTTACCTGATGT |
| | | AGGCAAATTTGGATCTAGTATGTTGGGGAAATAT |
| | | GAACCTGACATGATTAAGATTGCTCTTTCGGTGG |
| | | CTGGTAATTTAATAAGAAATCGAGACTACATTCC |
| | | CGGGAGACGAGGATATAGCTACTACGTTTACGGT |
| | | ATAGCCTCTAGATAATTTTTTTAAGCACGAAATA |
| | | AAAAACATAATTTTAAACCAATCTATTTCATACT |
| | | ATTTTGTGTGATCACCATGTACCTTGAGGTTAGT |
| | | GAACGTCAAGTGCTAGATGCCTCGGACTTTGC |
| | | CTTTCTTCTGGAAAACAGCACCTCTCCCTACGA |
| | | TTATGGGGAAAACGAGAGCGACTTCTCTGACT |
| | | CCCCGCCCTGCCCACAGGATTTCAGCCTGAAC |
| | | TTTGACAGAACCTTCCTGCCAGCCCTCTACAG |
| | | CCTCCTCTTCTTGCTGGGGCTGCTAGGCAATG |
| | | GGGCGGTGGCTGCTGTGCTACTGAGTCAGCGC |
| | | ACTGCCCTGAGCAGCACGGACACCTTCCTGCT |
| | | CCACCTGGCTGTAGCCGATGTTCTGCTGGTGT |
| | | TAACTCTTCCATTGTGGGCAGTGGATGCTGCT |
| | | GTCCAGTGGGTTTTCGGCCCTGGCCTCTGCAA |
| | | AGTGGCAGGCGCCTTGTTCAACATCAACTTCT |
| | | ATGCAGGGGCCTTCCTGCTGGCTTGTATAAGC |
| | | TTCGACAGATATCTGAGCATAGTGCACGCCAC |
| | | CCAGATCTACCGCAGGGACCCCCGGGTACGTG |
| | | TAGCCCTCACCTGCATAGTTGTATGGGGTCTC |
| | | TGTCTGCTCTTTGCCCTCCCAGATTTCATCTAC |
| | | CTATCAGCCAACTACGATCAGCGCCTCAATGC |
| | | CACCCATTGCCAGTACAACTTCCCACAGGTGG |
| | | GTCGCACTGCTCTGCGTGTACTGCAGCTAGTG |
| | | GCTGGTTTCCTGCTGCCCCTTCTGGTCATGGC |
| | | CTACTGCTATGCCCATATCCTAGCTGTTCTGCT |
| | | GGTCTCCAGAGGCCAGAGGCGTTTTCGAGCTA |
| | | TGAGGCTAGTGGTAGTGGTGGTGGCAGCCTTT |
| | | GCTGTCTGCTGGACCCCCTATCACCTGGTGGT |
| | | GCTAGTGGATATCCTCATGGATGTGGGAGTTT |
| | | TGGCCCGCAACTGTGGTCGAGAAAGCCACGTG |
| | | GATGTGGCCAAGTCAGTCACCTCGGGCATGGG |
| | | GTACATGCACTGCTGCCTCAATCCGCTGCTCT |
| | | ATGCCTTTGTGGGAGTGAAGTTCAGAGAGCAA |
| | | ATGTGGATGTTGTTCACGCGCCTGGGCCGCTC |
| | | TGACCAGAGAGGGCCCCAGCGGCAGCCGTCAT |
| | | CTTCACGGAGAGAATCATCCTGGTCTGAGACA |
| | | ACTGAGGCCTCCTACCTGGGCTTGTAATAGGA |
| | | GCTCTAGAATAACTTCGTATAGCATACATTATA |
| | | CGAAGTTATAAAGTTAAAAGTTAATGAGAGCAA |
| | | AAATATATAAGGTTGTATTCCATATTTGTTATTTT |
| | | TTCTGTAATAGTTAGAAAAATACATTCGATGGTC |
| | | TATCTACCAGATTATTATGTGTTATAAGGTACTTT |
| | | TTCTCATAATAAACTAGAGTATGAGTAAGATAGT |
| | | GTTTTTCAAAACATATAAATCTAAAATTGATGGA |
| | | TGAGATATACAGCTATTAATTTCGAAAATATATTT |
| | | TAATCTGATAACTTTAAACATGGATTTTTGATGGT |
| | | GGTTTAACGTTTTAAAAAAAGATTTTGTTATTGTA |
| | | GTATATGATAATATTAAAAGATGGATATAAAGAA |
| | | TTTGCTGACTGCATGTACTATTTTTTACATTACTA |
| | | CATTGGCTACGGCAGATATACCTACTCCGCCACC |
| | | AACGGGTCATGTGACAAGGGAGAATATCTTGATA |
| | | AGAGGCATAATCAATGTTGTAATCGGTGTCCACC |
| | | TGGAGAATTTGCCAAGGTTAGATGTAAT |

### Example 6: Construction of Murine IL-12 and TGF-Beta Inhibitor Expression System

The modified vaccinia virus of Example 5 was further modified by replacing the gene encoding thymidine kinase (VACV094, J2R) with nucleic acid encoding single-chain murine IL-12, a TGF-bf2, and a fluorescent reporter. A plasmid transfer vector was generated comprising, in order, with no gaps, an upstream recombination directing sequence B (SEQ ID NO: 76), an SbfI cloning site followed by P135 promoter (SEQ ID NO: 77), open reading frame encoding murine IL-12 (SEQ ID NO: 64), SalI cloning site followed by spacer (SEQ ID NO: 78), vaccinia virus promoter P7.5 followed by KpnI cloning site (SEQ ID NO: 79), open reading frame encoding TGF-beta inhibitor (TGFbf2) (SEQ ID NO: 67), a sacI cloning site (SEQ ID NO: 81), a loxP site (SEQ ID NO: 71), a spacer followed by viral promoter driving expression of GFP-pac reporter (SEQ ID NO: 72), a PacI cloning site and short spacer B (SEQ ID NO: 82), a loxP site (SEQ ID NO: 71), and a downstream recombination directing sequence B (SEQ ID NO: 83). Following recombination and treatment with cre recombinase, the viral genome incorporated the sequence as set forth in SEQ ID NO: 84. Selected sequences are set forth in Table 12. A schematic representation of the promoter and gene expression construct is shown in FIG. 6.

TABLE 12
Murine IL-12 and TGF-beta inhibitor recombination sequences.

| SEQ | | |
|---|---|---|
| ID NO: | Description | Nucleic acid sequence |
| 76 | Upstream recombination | ATTTACGGATTCACCAATAAAAATAAACTAGAG |
| | directing sequence B | AAACTTAGTACTAATAAGGAACTAGAATCGTAT |
| | | AGTTCTAGCCCTCTTCAAGAACCCATTAGGTTAA |
| | | ATGATTTTCTGGGACTATTGGAATGTGTTAAAAA |
| | | GAATATTCCTCTAACAGATATTCCGACAAAGGA |
| | | TTGATTACTATAAATGGAGAATGTTCCTAATGTA |
| | | TACTTTAATCCTGTGTTTATAGAGCCCACGTTTA |
| | | AACATTCTTTATTAAGTGTTTATAAACACAGATT |
| | | AATAGTTTTATTTGAAGTATTCGTTGTATTCATTC |
| | | TAATATATGTATTTTTTAGATCTGAATTAAATAT |
| | | GTTCTTCATGCCTAAACGAAAAATACCCGATCCT |
| | | ATTGATAGATTACGACGTGCTAATCTAGCGTGTG |
| | | AAGACGATAAATTAATGATCTATGGATTACCAT |
| | | GGATGACAACTCAAACATCTGCGTTATCAATAA |
| | | ATAGTAAACCGATAGTGTATAAAGATTGTGCAA |
| | | AGCTTTTGCGATCAATAAATGGATCACAACCAG |
| | | TATCTCTTAACGATGTTCTTCGCAGATGATGATT |
| | | CATTTTTTAAGTATTTGGCTAGTCAAGATGATGA |
| | | ATCTTCATTATCTGATATATTGCAAATCACTCAA |
| | | TATCTAGACTTTCTGTTATTATTATTGATCCAATC |
| | | AAAAAATAAATTAGAAGCCGTGGGTCATTGTTA |
| | | TGAATCTCTTTCAGAGGAATACAGACAATTGAC |
| | | AAAATTCACAGACTTTCAAGATTTTAAAAAACT |
| | | GTTTAACAAGGTCCCTATTGTTACAGATGGAAG |
| | | GGTCAAACTTAATAAAGGATATTTGTTCGACTTT |
| | | GTGATTAGTTTGATGCGATTCAAAAAAGAATCCT |
| | | CTCTAGCTACCACCGCAATAGATCCTGTTAGATA |
| | | CATAGATCCTCGTCGCAATATCGCATTTTCTAAC |
| | | GTGATGGATATATTAAAGTCGAATAAAGTGAAC |
| | | AATAATTAATTCTTTATTGTCATC |
| 77 | SbfI cloning site followed by | CCTGCAGGGCGTCCTAAAATTAAAACAAATGAT |
| | P135 promoter | ATAA |
| 78 | SalI cloning site followed by | GTCGACTGAGTGACCGTGA |
| | spacer | |
| 79 | Vaccinia virus promoter P7.5 | TCACTAATTCCAAACCCACCCGCTTTTTATAGTA |
| | followed by KpnI cloning site | AGTTTTTCACCCATAAATAATAAATACAATAATT |
| | | AATTTCTCGTAAAAGTAGAAAATATATTCTAATT |
| | | TATTGCACGGTAAGGAAGTAGATCATAAGGTAC |
| | | C |
| 67 | Open reading frame encoding | ATGGACTGGACATGGATTCTCTTTCTAGTGGCCG |
| | TGF-beta inhibitor, TGFbf2 | CAGCCACAAGGGTCCACAGCGGCCGCAAGGAAG |
| | | TCCTTGCTTCTAGTCAGGAAGCGCTCGTTGTAAC |
| | | AGAAAGAAAATATCTCAAATCCGACTGGTGTAA |
| | | GCTGCGACCACTCTACATAGATTTTCGAAAGGA |
| | | CCTGGGCTGGAAATGGATACATGAGCCAAAGGG |
| | | CTATAATGCGAATTTTTGCTACGGACAATGTAAT |
| | | TCTTTCTATATCCCCCGGCACGTCAAAAAGGAGG |
| | | AAGATTCTTTCCAATCATCTGCATTCTGTGTAAG |
| | | CCAGGATCTCGAGCCGTTGACTATTGTCTACTAC |
| | | GTTGGAAGAAAACCGAAGGTCGAGCAACTGTCT |
| | | AACATGATCGTCAAATCATGCAGGTGCATGTCA |
| | | GTGTGA |
| | SacI cloning site | GAGCTC |
| 82 | PacI cloning site and short | TTAATTAAAAGGCCGCTCGA |
| | spacer B | |
| 83 | Downstream recombination | TATTATATTTTTTATCTAAAAAACTAAAAATAAA |
| | directing sequence B | CATTGATTAAATTTTAATATAATACTTAAAAATG |
| | | GATGTTGTGTCGTTAGATAAACCGTTTATGTATT |
| | | TTGAGGAAATTGATAATGAGTTAGATTACGAAC |
| | | CAGAAAGTGCAAATGAGGTCGCAAAAAAACTGC |
| | | CGTATCAAGGACAGTTAAAACTATTACTAGGAG |
| | | AATTATTTTTTCTTAGTAAGTTACAGCGACACGG |
| | | TATATTAGATGGTGCCACCGTAGTGTATATAGGA |
| | | TCTGCTCCCGGTACACATATACGTTATTTGAGAG |
| | | ATCATTTCTATAATTTAGGAGTGATCATCAAATG |
| | | GATGCTAATTGACGGCCGCCATCATGATCCTATT |
| | | TTAAATGGATTGCGTGATGTGACTCTAGTGACTC |
| | | GGTTCGTTGATGAGGAATATCTACGATCCATCAA |
| | | AAAACAACTGCATCCTTCTAAGATTATTTTAATT |
| | | TCTGATGTGAGATCCAAACGAGGAGGAAATGAA |
| | | CCTAGTACGGCGGATTTACTAAGTAATTACGCTC |
| | | TACAAAATGTCATGATTAGTATTTTAAACCCCGT |
| | | GGCGTCTAGTCTTAAATGGAGATGCCCGTTTCCA |
| | | GATCAATGGATCAAGGACTTTTATATCCCACACG |
| | | GTAATAAAATGTTACAACCTTTTGCTCCTTCATA |
| | | TTCAGCTGAAATGAGATTATTAAGTATTTATACC |
| | | GGTGAGAACATGAGACTGACTCGAGTTACCAAA |
| | | TCAGACGCTGTAAATTATGAAAAAAAGATGTAC |
| | | TACCTTAATAAGATCGTCCGTAACAAAGTAGTTG |
| | | TTAACTTTGATTATCCTAATCAGGAATATGACTA |
| | | TTTTCACATGTACTTTATGCTGAGGACCGTGTAC |
| | | TGCAATAAAACATTTCCTACTACTAAAGCAAAG |
| | | GTACTATTTCTACAACAATCTATATTTCGTTTCTT |
| | | AAATATTCCAACAACATCAACTGAAAAAGTTAG |
| | | TCATGAACCAATACAACG |
| 84 | Sequence incorporated in viral | ATTTACGGATTCACCAATAAAAATAAACTAGAG |
| | genome (non-native DNA | AAACTTAGTACTAATAAGGAACTAGAATCGTAT |
| | BOLD; murine IL-12 and | AGTTCTAGCCCTCTTCAAGAACCCATTAGGTTAA |
| | TGF-bf2 open reading frames | ATGATTTTCTGGGACTATTGGAATGTGTTAAAAA |
| | UNDERLINED) | GAATATTCCTCTAACAGATATTCCGACAAAGGA |
| | | TTGATTACTATAAATGGAGAATGTTCCTAATGTA |
| | | TACTTTAATCCTGTGTTTATAGAGCCCACGTTTA |
| | | AACATTCTTTATTAAGTGTTTATAAACACAGATT |
| | | AATAGTTTTATTTGAAGTATTCGTTGTATTCATTC |
| | | TAATATATGTATTTTTTAGATCTGAATTAAATAT |
| | | GTTCTTCATGCCTAAACGAAAAATACCCGATCCT |
| | | ATTGATAGATTACGACGTGCTAATCTAGCGTGTG |
| | | AAGACGATAAATTAATGATCTATGGATTACCAT |
| | | GGATGACAACTCAAACATCTGCGTTATCAATAA |
| | | ATAGTAAACCGATAGTGTATAAAGATTGTGCAA |
| | | AGCTTTTGCGATCAATAAATGGATCACAACCAG |
| | | TATCTCTTAACGATGTTCTTCGCAGATGATGATT |
| | | CATTTTTTAAGTATTTGGCTAGTCAAGATGATGA |
| | | ATCTTCATTATCTGATATATTGCAAATCACTCAA |
| | | TATCTAGACTTTCTGTTATTATTATTGATCCAATC |
| | | AAAAAATAAATTAGAAGCCGTGGGTCATTGTTA |
| | | TGAATCTCTTTCAGAGGAATACAGACAATTGAC |
| | | AAAATTCACAGACTTTCAAGATTTTAAAAAACT |
| | | GTTTAACAAGGTCCCTATTGTTACAGATGGAAG |
| | | GGTCAAACTTAATAAAGGATATTTGTTCGACTTT |
| | | GTGATTAGTTTGATGCGATTCAAAAAAGAATCCT |
| | | CTCTAGCTACCACCGCAATAGATCCTGTTAGATA |
| | | CATAGATCCTCGTCGCAATATCGCATTTTCTAAC |
| | | GTGATGGATATATTAAAGTCGAATAAAGTGAAC |
| | | AATAATTAATTCTTTATTGTCATCCCTGCAGGG |
| | | CGTCCTAAAATTAAAACAAATGATATAAATGT |
| | | GTCCTCAGAAGCTAACCATCTCCTGGTTTGCC |
| | | ATCGTTTTGCTGGTGTCTCCACTCATGGCCAT |
| | | GTGGGAGCTGGAGAAAGACGTTTATGTTGTA |
| | | GAGGTGGACTGGACTCCCGATGCCCCTGGAG |
| | | AAACAGTGAACCTCACCTGTGACACGCCTGA |
| | | AGAAGATGACATCACCTGGACCTCAGACCAG |
| | | AGACATGGAGTCATAGGCTCTGGAAAGACCC |
| | | TGACCATCACTGTCAAAGAGTTTCTAGATGCT |
| | | GGCCAGTACACCTGCCACAAAGGAGGCGAGA |
| | | CTCTGAGCCACTCACATCTGCTGCTCCACAAG |
| | | AAGGAAAATGGAATTTGGTCCACTGAAATTTT |
| | | AAAAAATTTCAAAAACAAGACTTTCCTGAAGT |
| | | GTGAAGCACCAAATTACTCCGGACGGTTCAC |
| | | GTGCTCATGGCTGGTGCAAAGAAACATGGAC |
| | | TTGAAGTTCAACATCAAGAGCAGTAGCAGTTC |
| | | CCCTGACTCTCGGGCAGTGACATGTGGAATG |
| | | GCGTCTCTGTCTGCAGAGAAGGTCACACTGG |
| | | ACCAAAGGGACTATGAGAAGTATTCAGTGTC |
| | | CTGCCAGGAGGATGTCACCTGCCCAACTGCC |
| | | GAGGAGACCCTGCCCATTGAACTGGCGTTGG |
| | | AAGCACGGCAGCAGAATAAATATGAGAACTA |
| | | CAGCACCAGCTTCTTCATCAGGGACATCATCA |
| | | AACCAGACCCGCCCAAGAACTTGCAGATGAA |
| | | GCCTTTGAAGAACTCACAGGTGGAGGTCAGC |
| | | TGGGAGTACCCTGACTCCTGGAGCACTCCCC |
| | | ATTCCTACTTCTCCCTCAAGTTCTTTGTTCGA |
| | | ATCCAGCGCAAGAAAGAAAAGATGAAGGAGA |
| | | CAGAGGAGGGGTGTAACCAGAAAGGTGCGTT |
| | | CCTCGTAGAGAAGACATCTACCGAAGTCCAAT |
| | | GCAAAGGCGGGAATGTCTGCGTGCAAGCTCA |
| | | GGATCGCTATTACAATTCCTCATGCAGCAAGT |
| | | GGGCATGTGTTCCATGCAGGGTCCGATCCGG |
| | | AGGTGGCGGATCCGGCGGAGGTGGCTCAGGT |
| | | GGCGGTGGATCAGGAGGAGGTGGATCAGCTA |
| | | GCGCCACCCTTGCCCTCCTAAACCACCTCAGT |
| | | TTGGCCAGGGTCATTCCAGTCTCTGGACCTGC |
| | | CAGGTGTCTTAGCCAGTCCCGAAACCTGCTG |
| | | AAGACCACAGATGACATGGTGAAGACGGCCA |
| | | GAGAAAAACTGAAACATTATTCCTGCACTGCT |
| | | GAAGACATCGACCATGAAGACATCACACGGG |
| | | ACCAAACCAGCACATTGAAGACCTGTTTACCA |
| | | CTGGAACTACACAAGAACGAGAGTTGCCTGG |
| | | CTACTAGAGAGACTTCTTCCACAACAAGAGG |
| | | GAGCTGCCTGCCCCCACAGAAGACGTCTTTG |
| | | ATGATGACCCTGTGCCTTGGTAGCATCTATGA |
| | | GGACTTGAAGATGTACCAGACAGAGTTCCAG |
| | | GCCATCAACGCAGCACTTCAGAATCACAACCA |
| | | TCAGCAGATCATTCTAGACAAGGGCATGCTG |
| | | GTGGCCATCGACGAGCTGATGCAGTCTCTGA |
| | | ATCATAATGGCGAGACTCTGCGCCAGAAACC |
| | | TCCTGTGGGAGAAGCAGACCCTTACAGAGTG |
| | | AAAATGAAGCTCTGCATCCTGCTTCACGCCTT |
| | | CAGCACCCGCGTCGTGACCATCAACAGGGTG |
| | | ATGGGCTATCTGAGTTCCGCCTAAGTCGACTG |
| | | AGTGACCGTGATCACTAATTCCAAACCCACCC |
| | | GCTTTTTATAGTAAGTTTTTCACCCATAAATA |
| | | ATAAATACAATAATTAATTTCTCGTAAAAGTA |
| | | GAAAATATATTCTAATTTATTGCACGGTAAGG |
| | | AAGTAGATCATAAGGTACCATGGACTGGACA |
| | | TGGATTCTCTTTCTAGTGGCCGCAGCCACAAG |
| | | GGTCCACAGCGGCCGCAAGGAAGTCCTTGCT |
| | | TCTAGTCAGGAAGCGCTCGTTGTAACAGAAA |
| | | GAAAATATCTCAAATCCGACTGGTGTAAGCTG |
| | | CGACCACTCTACATAGATTTTCGAAAGGACCT |
| | | GGGCTGGAAATGGATACATGAGCCAAAGGGC |
| | | TATAATGCGAATTTTTGCTACGGACAATGTAA |
| | | TTCTTTCTATATCCCCCGGCACGTCAAAAAGG |
| | | AGGAAGATTCTTTCCAATCATCTGCATTCTGT |
| | | GTAAGCCAGGATCTCGAGCCGTTGACTATTGT |
| | | CTACTACGTTGGAAGAAAACCGAAGGTCGAG |
| | | CAACTGTCTAACATGATCGTCAAATCATGCAG |
| | | GTGCATGTCAGTGTGAGAGCTCATAACTTCGT |
| | | ATAGCATACATTATACGAAGTTATTATTATATT |
| | | TTTTATCTAAAAAACTAAAAATAAACATTGATTA |
| | | AATTTTAATATAATACTTAAAAATGGATGTTGTG |
| | | TCGTTAGATAAACCGTTTATGTATTTTGAGGAAA |
| | | TTGATAATGAGTTAGATTACGAACCAGAAAGTG |
| | | CAAATGAGGTCGCAAAAAAACTGCCGTATCAAG |
| | | GACAGTTAAAACTATTACTAGGAGAATTATTTTT |
| | | TCTTAGTAAGTTACAGCGACACGGTATATTAGAT |
| | | GGTGCCACCGTAGTGTATATAGGATCTGCTCCCG |
| | | GTACACATATACGTTATTTGAGAGATCATTTCTA |
| | | TAATTTAGGAGTGATCATCAAATGGATGCTAATT |
| | | GACGGCCGCCATCATGATCCTATTTTAAATGGAT |
| | | TGCGTGATGTGACTCTAGTGACTCGGTTCGTTGA |
| | | TGAGGAATATCTACGATCCATCAAAAAACAACT |
| | | GCATCCTTCTAAGATTATTTTAATTTCTGATGTG |
| | | AGATCCAAACGAGGAGGAAATGAACCTAGTACG |
| | | GCGGATTTACTAAGTAATTACGCTCTACAAAATG |
| | | TCATGATTAGTATTTTAAACCCCGTGGCGTCTAG |
| | | TCTTAAATGGAGATGCCCGTTTCCAGATCAATGG |
| | | ATCAAGGACTTTTATATCCCACACGGTAATAAA |
| | | ATGTTACAACCTTTTGCTCCTTCATATTCAGCTG |
| | | AAATGAGATTATTAAGTATTTATACCGGTGAGA |
| | | ACATGAGACTGACTCGAGTTACCAAATCAGACG |
| | | CTGTAAATTATGAAAAAAAGATGTACTACCTTA |
| | | ATAAGATCGTCCGTAACAAAGTAGTTGTTAACTT |
| | | TGATTATCCTAATCAGGAATATGACTATTTTCAC |
| | | ATGTACTTTATGCTGAGGACCGTGTACTGCAATA |
| | | AAACATTTCCTACTACTAAAGCAAAGGTACTATT |
| | | TCTACAACAATCTATATTTCGTTTCTTAAATATTC |
| | | CAACAACATCAACTGAAAAAGTTAGTCATGAAC |
| | | CAATACAACG |

### Example 7: Construction of Human IL-12 and TGF-Beta Inhibitor Expression System

A vaccinia virus was modified by replacing the gene encoding thymidine kinase (VACV094, J2R) with nucleic acid encoding single-chain human IL-12, a TGF-β inhibitor TGFbf2, and a fluorescent reporter. A plasmid transfer vector was generated comprising, in order, with no gaps, a upstream recombination directing sequence B (SEQ ID NO: 76), an SbfI cloning site followed by P135 promoter (SEQ ID NO: 77), open reading frame encoding human IL-12 (SEQ ID NO: 66), SalI cloning site followed by spacer (SEQ ID NO: 78), vaccinia virus promoter P7.5 followed by KpnI cloning site (SEQ ID NO: 79), open reading frame encoding TGF-beta inhibitor (TGFbf2) (SEQ ID NO: 67), a sacI cloning site (SEQ ID NO: 81), a loxP site (SEQ ID NO: 71), a spacer followed by viral promoter driving expression of GFP-pac reporter (SEQ ID NO: 72), a PacI cloning site and short spacer B (SEQ ID NO: 82), a loxP site (SEQ ID NO: 71), and a downstream recombination directing sequence B (SEQ ID NO: 83). Following recombination and treatment with cre recombinase, the viral genome comprised the incorporated sequence as set forth in SEQ ID NO: 85. Sequences are set forth in Table 13.

TABLE 13
Human IL-12 and TGF-beta inhibitor recombination sequences.

| SEQ ID | | |
|---|---|---|
| NO: | Description | Nucleic acid sequence |
| 85 | Sequence incorporated in | ATTTACGGATTCACCAATAAAAATAAACTAGAG |
| | viral genome (non-native | AAACTTAGTACTAATAAGGAACTAGAATCGTAT |
| | DNA BOLD; human IL-12 | AGTTCTAGCCCTCTTCAAGAACCCATTAGGTTA |
| | and TGF-bf2 open reading | AATGATTTTCTGGGACTATTGGAATGTGTTAAA |
| | frames UNDERLINED) | AAGAATATTCCTCTAACAGATATTCCGACAAAG |
| | | GATTGATTACTATAAATGGAGAATGTTCCTAAT |
| | | GTATACTTTAATCCTGTGTTTATAGAGCCCACGT |
| | | TTAAACATTCTTTATTAAGTGTTTATAAACACAG |
| | | ATTAATAGTTTTATTTGAAGTATTCGTTGTATTC |
| | | ATTCTAATATATGTATTTTTTAGATCTGAATTAA |
| | | ATATGTTCTTCATGCCTAAACGAAAAATACCCG |
| | | ATCCTATTGATAGATTACGACGTGCTAATCTAG |
| | | CGTGTGAAGACGATAAATTAATGATCTATGGAT |
| | | TACCATGGATGACAACTCAAACATCTGCGTTAT |
| | | CAATAAATAGTAAACCGATAGTGTATAAAGATT |
| | | GTGCAAAGCTTTTGCGATCAATAAATGGATCAC |
| | | AACCAGTATCTCTTAACGATGTTCTTCGCAGAT |
| | | GATGATTCATTTTTTAAGTATTTGGCTAGTCAAG |
| | | ATGATGAATCTTCATTATCTGATATATTGCAAAT |
| | | CACTCAATATCTAGACTTTCTGTTATTATTATTG |
| | | ATCCAATCAAAAAATAAATTAGAAGCCGTGGGT |
| | | CATTGTTATGAATCTCTTTCAGAGGAATACAGA |
| | | CAATTGACAAAATTCACAGACTTTCAAGATTTT |
| | | AAAAAACTGTTTAACAAGGTCCCTATTGTTACA |
| | | GATGGAAGGGTCAAACTTAATAAAGGATATTTG |
| | | TTCGACTTTGTGATTAGTTTGATGCGATTCAAAA |
| | | AAGAATCCTCTCTAGCTACCACCGCAATAGATC |
| | | CTGTTAGATACATAGATCCTCGTCGCAATATCG |
| | | CATTTTCTAACGTGATGGATATATTAAAGTCGA |
| | | ATAAAGTGAACAATAATTAATTCTTTTTTGTCAT |
| | | CCCTGCAGGGCGTCCTAAAATTAAAACAAAT |
| | | GATATAAATGTGTCACCAGCAGTTGGTCATC |
| | | TCTTGGTTTTCCCTGGTTTTTCTGGCATCTCC |
| | | CCTCGTGGCCATATGGGAACTGAAGAAAGAT |
| | | GTTTATGTCGTAGAATTGGATTGGTATCCGG |
| | | ATGCCCCTGGAGAAATGGTGGTCCTCACCTG |
| | | TGACACCCCTGAAGAAGATGGTATCACCTGG |
| | | ACCTTGGACCAGAGCAGTGAGGTCTTAGGCT |
| | | CTGGCAAAACCCTGACCATCCAAGTCAAAGA |
| | | GTTTGGAGATGCTGGCCAGTACACCTGTCAC |
| | | AAAGGAGGCGAGGTTCTAAGCCATTCGCTCC |
| | | TGCTGCTTCACAAAAAGGAAGATGGAATTTG |
| | | GTCCACTGATATTTTAAAGGACCAGAAAGAA |
| | | CCCAAAAATAAGACCTTTCTAAGATGCGAGG |
| | | CCAAGAATTATTCTGGACGTTTCACCTGCTG |
| | | GTGGCTGACGACAATCAGTACTGATTTGACA |
| | | TTCAGTGTCAAAAGCAGCAGAGGCTCTTCTG |
| | | ACCCCCAAGGGGTGACGTGCGGAGCTGCTAC |
| | | ACTCTCTGCAGAGAGAGTCAGAGGGGACAAC |
| | | AAGGAGTATGAGTACTCAGTGGAGTGCCAGG |
| | | AGGACAGTGCCTGCCCAGCTGCTGAGGAGA |
| | | GTCTGCCCATTGAGGTCATGGTGGATGCCGT |
| | | TCACAAGCTCAAGTATGAAAACTACACCAGC |
| | | AGCTTCTTCATCAGGGACATCATCAAACCTG |
| | | ACCCACCCAAGAACTTGCAGCTGAAGCCATT |
| | | AAAGAATTCTCGGCAGGTGGAGGTCAGCTGG |
| | | GAGTACCCTGACACCTGGAGTACTCCACATT |
| | | CCTACTTCTCCCTGACATTCTGCGTTCAGGTC |
| | | CAGGGCAAGAGCAAGAGAGAAAAGAAAGAT |
| | | AGAGTCTTCACGGACAAGACCTCAGCCACGG |
| | | TCATCTGCCGCAAAAATGCCAGCATTAGCGT |
| | | GCGGGCCCAGGACCGCTACTATAGCTCATCT |
| | | TGGAGCGAATGGGCATCTGTGCCCTGCAGTG |
| | | GAGGTGGCGGATCCGGCGGAGGTGGCTCAG |
| | | GTGGCGGTGGATCAGGAGGAGGTGGATCAG |
| | | CTAGCTGGCCCCCTGGGTCAGCCTCCCAGCC |
| | | ACCGCCCTCACCTGCCGCGGCCACAGGTCTG |
| | | CATCCAGCGGCTCGCCCTGTGTCCCTGCAGT |
| | | GCCGGCTCAGCATGTGTCCAGCGCGCAGCCT |
| | | CCTCCTTGTGGCTACCCTGGTCCTCCTGGAC |
| | | CACCTCAGTTTGGCCAGAAACCTCCCCGTGG |
| | | CCACTCCAGACCCAGGAATGTTCCCATGCCT |
| | | TCACCACTCCCAAAACCTGCTGAGGGCCGTC |
| | | AGCAACATGCTCCAGAAGGCCAGACAAACTC |
| | | TAGAATTTTACCCTTGCACTTCTGAAGAGATT |
| | | GATCATGAAGATATCACAAAAGATAAAACCA |
| | | GCACAGTGGAGGCCTGTTTACCATTGGAATT |
| | | AACCAAGAATGAGAGTTGCCTAAATTCCAGA |
| | | GAGACCTCTTTCATAACTAATGGGAGTTGCC |
| | | TGGCCTCCAGAAAGACCTCTTTTATGATGGC |
| | | CCTGTGCCTTAGTAGTATTTATGAAGACTTG |
| | | AAGATGTACCAGGTGGAGTTCAAGACCATGA |
| | | ATGCAAAGCTTCTGATGGATCCTAAGAGGCA |
| | | GATCTTTCTAGATCAAAACATGCTGGCAGTT |
| | | ATTGATGAGCTGATGCAGGCCCTGAATTTCA |
| | | ACAGTGAGACTGTGCCACAAAAATCCTCCCT |
| | | TGAAGAACCGGATTTTTATAAAACTAAAATC |
| | | AAGCTCTGCATACTTCTTCATGCTTTCAGAAT |
| | | TCGGGCAGTGACTATTGATAGAGTGATGAGC |
| | | TATCTGAATGCTTCCTAAGTCGACTGAGTGA |
| | | CCGTGATCACTAATTCCAAACCCACCCGCTT |
| | | TTTATAGTAAGTTTTTCACCCATAAATAATAA |
| | | ATACAATAATTAATTTCTCGTAAAAGTAGAAA |
| | | ATATATTCTAATTTATTGCACGGTAAGGAAGT |
| | | AGATCATAAGGTACCATGGACTGGACATGGA |
| | | TTCTCTTTCTAGTGGCCGCAGCCACAAGGGT |
| | | CCACAGCGGCCGCAAGGAAGTCCTTGCTTCT |
| | | AGTCAGGAAGCGCTCGTTGTAACAGAAAGAA |
| | | AATATCTCAAATCCGACTGGTGTAAGCTGCG |
| | | ACCACTCTACATAGATTTTCGAAAGGACCTG |
| | | GGCTGGAAATGGATACATGAGCCAAAGGGCT |
| | | ATAATGCGAATTTTTGCTACGGACAATGTAA |
| | | TTCTTTCTATATCCCCCGGCACGTCAAAAAG |
| | | GAGGAAGATTCTTTCCAATCATCTGCATTCT |
| | | GTGTAAGCCAGGATCTCGAGCCGTTGACTAT |
| | | TGTCTACTACGTTGGAAGAAAACCGAAGGTC |
| | | GAGCAACTGTCTAACATGATCGTCAAATCAT |
| | | GCAGGTGCATGTCAGTGTGAGAGCTCATAAC |
| | | TTCGTATAGCATACATTATACGAAGTTATTAT |
| | | TATATTTTTTATCTAAAAAACTAAAAATAAACA |
| | | TTGATTAAATTTTAATATAATACTTAAAAATGG |
| | | ATGTTGTGTCGTTAGATAAACCGTTTATGTATTT |
| | | TGAGGAAATTGATAATGAGTTAGATTACGAACC |
| | | AGAAAGTGCAAATGAGGTCGCAAAAAAACTGC |
| | | CGTATCAAGGACAGTTAAAACTATTACTAGGAG |
| | | AATTATTTTTTCTTAGTAAGTTACAGCGACACG |
| | | GTATATTAGATGGTGCCACCGTAGTGTATATAG |
| | | GATCTGCTCCCGGTACACATATACGTTATTTGA |
| | | GAGATCATTTCTATAATTTAGGAGTGATCATCA |
| | | AATGGATGCTAATTGACGGCCGCCATCATGATC |
| | | CTATTTTAAATGGATTGCGTGATGTGACTCTAGT |
| | | GACTCGGTTCGTTGATGAGGAATATCTACGATC |
| | | CATCAAAAAACAACTGCATCCTTCTAAGATTAT |
| | | TTTAATTTCTGATGTGAGATCCAAACGAGGAGG |
| | | AAATGAACCTAGTACGGCGGATTTACTAAGTAA |
| | | TTACGCTCTACAAAATGTCATGATTAGTATTTTA |
| | | AACCCCGTGGCGTCTAGTCTTAAATGGAGATGC |
| | | CCGTTTCCAGATCAATGGATCAAGGACTTTTAT |
| | | ATCCCACACGGTAATAAAATGTTACAACCTTTT |
| | | GCTCCTTCATATTCAGCTGAAATGAGATTATTA |
| | | AGTATTTATACCGGTGAGAACATGAGACTGACT |
| | | CGAGTTACCAAATCAGACGCTGTAAATTATGAA |
| | | AAAAAGATGTACTACCTTAATAAGATCGTCCGT |
| | | AACAAAGTAGTTGTTAACTTTGATTATCCTAAT |
| | | CAGGAATATGACTATTTTCACATGTACTTTATGC |
| | | TGAGGACCGTGTACTGCAATAAAACATTTCCTA |
| | | CTACTAAAGCAAAGGTACTATTTCTACAACAAT |
| | | CTATATTTCGTTTCTTAAATATTCCAACAACATC |
| | | AACTGAAAAAGTTAGTCATGAACCAATACAAC |
| | | G |

### Example 8: Chemokine Receptor Sequence Addition

The modified vaccinia virus described in Example 7 was further modified by replacing the gene encoding A52 (VACV178, A52R) with nucleic acid encoding human CXCR3 and a fluorescent reporter. A plasmid transfer vector was generated comprising, in order, with no gaps, an upstream recombination directing sequence A (SEQ ID NO: 68), an open reading frame encoding human CXCR3 isoform 1 (SEQ ID NO: 86), a stop codon, SacI cloning site, and short spacer (SEQ ID NO: 70), a loxP site (SEQ ID NO: 71), a spacer followed by viral promoter driving expression of GFP-pac reporter (SEQ ID NO: 72), a PacI cloning site and short spacer A (SEQ ID NO: 73), a loxP site (SEQ ID NO: 71), and a downstream recombination directing sequence A (SEQ ID NO: 74). Reporter-positive viruses were isolated, then treated with a reporter-free transfer vector comprising an upstream recombination-directing sequence A (SEQ ID NO: 68), an open reading frame encoding human CXCR3 isoform 1 (SEQ ID NO: 86), and a second downstream recombination-directing sequence (SEQ ID NO: 87). Following recombination, the viral genome incorporated the sequence as set forth in SEQ ID NO: 88. Selected sequences are set forth in Table 14.

TABLE 14
Human CXCR3 recombination sequences.

| SEQ ID | | |
|---|---|---|
| NO: | Description | Nucleic acid sequence |
| 86 | Open reading frame encoding | ATGGTCCTTGAGGTGAGTGACCACCAAGTGCTA |
| | human CXCR3 isoform 1 | AATGACGCCGAGGTTGCCGCCCTCCTGGAGAAC |
| | | TTCAGCTCTTCCTATGACTATGGAGAAAACGAG |
| | | AGTGACTCGTGCTGTACCTCCCCGCCCTGCCCAC |
| | | AGGACTTCAGCCTGAACTTCGACCGGGCCTTCCT |
| | | GCCAGCCCTCTACAGCCTCCTCTTTCTGCTGGGG |
| | | CTGCTGGGCAACGGCGCGGTGGCAGCCGTGCTG |
| | | CTGAGCCGGCGGACAGCCCTGAGCAGCACCGAC |
| | | ACCTTCCTGCTCCACCTAGCTGTAGCAGACACGC |
| | | TGCTGGTGCTGACACTGCCGCTCTGGGCAGTGG |
| | | ACGCTGCCGTCCAGTGGGTCTTTGGCTCTGGCCT |
| | | CTGCAAAGTGGCAGGTGCCCTCTTCAACATCAA |
| | | CTTCTACGCAGGAGCCCTCCTGCTGGCCTGCATC |
| | | AGCTTTGACCGCTACCTGAACATAGTTCATGCCA |
| | | CCCAGCTCTACCGCCGGGGGCCCCCGGCCCGCG |
| | | TGACCCTCACCTGCCTGGCTGTCTGGGGGCTCTG |
| | | CCTGCTTTTCGCCCTCCCAGACTTCATCTTCCTGT |
| | | CGGCCCACCACGACGAGCGCCTCAACGCCACCC |
| | | ACTGCCAATACAACTTCCCACAGGTGGGCCGCA |
| | | CGGCTCTGCGGGTGCTGCAGCTGGTGGCTGGCTT |
| | | TCTGCTGCCCCTGCTGGTCATGGCCTACTGCTAT |
| | | GCCCACATCCTGGCCGTGCTGCTGGTTTCCAGGG |
| | | GCCAGCGGCGCCTGCGGGCCATGCGGCTGGTGG |
| | | TGGTGGTCGTGGTGGCCTTTGCCCTCTGCTGGAC |
| | | CCCCTATCACCTGGTGGTGCTGGTGGACATCCTC |
| | | ATGGACCTGGGCGCTTTGGCCCGCAACTGTGGC |
| | | CGAGAAAGCAGGGTAGACGTGGCCAAGTCGGTC |
| | | ACCTCAGGCCTGGGCTACATGCACTGCTGCCTCA |
| | | ACCCGCTGCTCTATGCCTTTGTAGGGGTCAAGTT |
| | | CCGGGAGCGGATGTGGATGCTGCTCTTGCGCCT |
| | | GGGCTGCCCCAACCAGAGAGGGCTCCAGAGGCA |
| | | GCCATCGTCTTCCCGCCGGGATTCATCCTGGTCT |
| | | GAGACCTCAGAGGCCTCCTACTCGGGCTTGTGA |
| 87 | Second downstream | AAAGTTAAAAGTTAATGAGAGCAAAAATATATA |
| | recombination-directing | AGGTTGTATTCCATATTTGTTATTTTTTCTGTAAT |
| | sequence | AGTTAGAAAAATACATTCGATGGTCTATCTACCA |
| | | GATTATTATGTGTTATAAGGTACTTTTTCTCATA |
| | | ATAAACTAGAGTATGAGTAAGATAGTGTTTTTCA |
| | | AAACATATAAATCTAAAATTGATGGATGAGATA |
| | | TACAGCTATTAATTTCGAAAATATATTTTAATCT |
| | | GATAACTTTAAACATGGATTTTTGATGGTGGTTT |
| | | AACGTTTTAAAAAAAGATTTTGTTATTGTAGTAT |
| | | ATGATAATATTAAAAGATGGATATAAAGAATTT |
| | | GCTGACTGCATGTACTATTTTTTACATTACTACA |
| | | TTGGCTACGGCAGATATACCTACTCCGCCACCAA |
| | | CGGGTCATGTGACAAGGGAGAATATCTTGATAA |
| | | GAGGCATAATCAATGTTGTAATCGGTGTCCACCT |
| | | GGAGAATTTGCCAAGGTTAGATGTAATGGTAAC |
| | | GATAACACAAAATGTGAACGCTGCCCACCTCAT |
| | | ACATATACCACAATCCCAATTATTCTAATGGATG |
| | | TCATCAATGTAGAAAATGCCCAACCGGATCATTT |
| | | GATAAGGTAAAGTGTACCGGAACACAGAACAGT |
| | | AAATGTTCGTGTCTTCCTGGTTGGTATTGTGCTA |
| | | CTGATTCTTCACAGACTGAAGATTGTTGAAATTG |
| | | TGTACCAAAAAGGAGATGTCCATGCGGATACTT |
| | | TGGTGGAATAGATGAACAAGGAAATCCTATTTG |
| | | TAAATCGTGTTGTGTTGGTGAATATTGCGACTAC |
| | | CTACGTAATTATAGACTTGATCCATTTCCTCCAT |
| | | GCAAACTATCTA |
| 88 | Sequence incorporated in | CTCATCAGACATATCGTGTGCATGCGTGGAGTTA |
| | viral genome (non-native | AATTCATATCACGTAGCCATTCTTCCCATGGATG |
| | DNA BOLD; CXCR3 | TTTCCTTTTTTACCAAAGGAAATGCATCATTGAT |
| | transgene UNDERLINED) | TATTCTCCTGTTTGATTTCTCTATCGATGCAGCAC |
| | | CTCTCTTAAGAAGTGTAACCGATAATAATGTTAT |
| | | TATATCTAGACACCAGCGTCTACATGACGAGCTT |
| | | CCGAGTTCCAATTGGTTCAAGTTTTACATAAGTA |
| | | TAAAGTCCGACTATTGTTCTATATTATATATGGT |
| | | TGTTGATGGATCTGTGATGCATGCGATAGCTGAT |
| | | AATAGAACTCACGCAATTATTAGCAAAAATATA |
| | | TTAGACAATACTACGATTAACGATGAGTGTAGA |
| | | TGCTGTTATTTTGAACCACAGATTAGGATTCTTG |
| | | ATAGAGATGAGATGCTCAATGGATCATCGTGTG |
| | | ATATGAACAGACATTGTATTATGATGAATTTACC |
| | | TGATGTAGGCAAATTTGGATCTAGTATGTTGGGG |
| | | AAATATGAACCTGACATGATTAAGATTGCTCTTT |
| | | CGGTGGCTGGTAATTTAATAAGAAATCGAGACT |
| | | ACATTCCCGGGAGACGAGGATATAGCTACTACG |
| | | TTTACGGTATAGCCTCTAGATAATTTTTTTAAGC |
| | | ACGAAATAAAAAACATAATTTTAAACCAATCTA |
| | | TTTCATACTATTTTGTGTGATCACCATGGTCCTT |
| | | GAGGTGAGTGACCACCAAGTGCTAAATGACG |
| | | CCGAGGTTGCCGCCCTCCTGGAGAACTTCAG |
| | | CTCTTCCTATGACTATGGAGAAAACGAGAGTG |
| | | ACTCGTGCTGTACCTCCCCGCCCTGCCCACAG |
| | | GACTTCAGCCTGAACTTCGACCGGGCCTTCCT |
| | | GCCAGCCCTCTACAGCCTCCTCTTTCTGCTGG |
| | | GGCTGCTGGGCAACGGCGCGGTGGCAGCCGT |
| | | GCTGCTGAGCCGGCGGACAGCCCTGAGCAGC |
| | | ACCGACACCTTCCTGCTCCACCTAGCTGTAGC |
| | | AGACACGCTGCTGGTGCTGACACTGCCGCTC |
| | | TGGGCAGTGGACGCTGCCGTCCAGTGGGTCT |
| | | TTGGCTCTGGCCTCTGCAAAGTGGCAGGTGC |
| | | CCTCTTCAACATCAACTTCTACGCAGGAGCCC |
| | | TCCTGCTGGCCTGCATCAGCTTTGACCGCTAC |
| | | CTGAACATAGTTCATGCCACCCAGCTCTACCG |
| | | CCGGGGGCCCCCGGCCCGCGTGACCCTCACC |
| | | TGCCTGGCTGTCTGGGGGCTCTGCCTGCTTTT |
| | | CGCCCTCCCAGACTTCATCTTCCTGTCGGCCC |
| | | ACCACGACGAGCGCCTCAACGCCACCCACTG |
| | | CCAATACAACTTCCCACAGGTGGGCCGCACG |
| | | GCTCTGCGGGTGCTGCAGCTGGTGGCTGGCT |
| | | TTCTGCTGCCCCTGCTGGTCATGGCCTACTGC |
| | | TATGCCCACATCCTGGCCGTGCTGCTGGTTTC |
| | | CAGGGGCCAGCGGCGCCTGCGGGCCATGCGG |
| | | CTGGTGGTGGTGGTCGTGGTGGCCTTTGCCC |
| | | TCTGCTGGACCCCCTATCACCTGGTGGTGCTG |
| | | GTGGACATCCTCATGGACCTGGGCGCTTTGG |
| | | CCCGCAACTGTGGCCGAGAAAGCAGGGTAGA |
| | | CGTGGCCAAGTCGGTCACCTCAGGCCTGGGC |
| | | TACATGCACTGCTGCCTCAACCCGCTGCTCTA |
| | | TGCCTTTGTAGGGGTCAAGTTCCGGGAGCGG |
| | | ATGTGGATGCTGCTCTTGCGCCTGGGCTGCC |
| | | CCAACCAGAGAGGGCTCCAGAGGCAGCCATC |
| | | GTCTTCCCGCCGGGATTCATCCTGGTCTGAGA |
| | | CCTCAGAGGCCTCCTACTCGGGCTTGTGAAA |
| | | AGTTAAAAGTTAATGAGAGCAAAAATATATAAG |
| | | GTTGTATTCCATATTTGTTATTTTTTCTGTAATAG |
| | | TTAGAAAAATACATTCGATGGTCTATCTACCAGA |
| | | TTATTATGTGTTATAAGGTACTTTTTCTCATAATA |
| | | AACTAGAGTATGAGTAAGATAGTGTTTTTCAAA |
| | | ACATATAAATCTAAAATTGATGGATGAGATATA |
| | | CAGCTATTAATTTCGAAAATATATTTTAATCTGA |
| | | TAACTTTAAACATGGATTTTTGATGGTGGTTTAA |
| | | CGTTTTAAAAAAAGATTTTGTTATTGTAGTATAT |
| | | GATAATATTAAAAGATGGATATAAAGAATTTGC |
| | | TGACTGCATGTACTATTTTTTACATTACTACATT |
| | | GGCTACGGCAGATATACCTACTCCGCCACCAAC |
| | | GGGTCATGTGACAAGGGAGAATATCTTGATAAG |
| | | AGGCATAATCAATGTTGTAATCGGTGTCCACCTG |
| | | GAGAATTTGCCAAGGTTAGATGTAATGGTAACG |
| | | ATAACACAAAATGTGAACGCTGCCCACCTCATA |
| | | CATATACCACAATCCCAATTATTCTAATGGATGT |
| | | CATCAATGTAGAAAATGCCCAACCGGATCATTT |
| | | GATAAGGTAAAGTGTACCGGAACACAGAACAGT |
| | | AAATGTTCGTGTCTTCCTGGTTGGTATTGTGCTA |
| | | CTGATTCTTCACAGACTGAAGATTGTTGAAATTG |
| | | TGTACCAAAAAGGAGATGTCCATGCGGATACTT |
| | | TGGTGGAATAGATGAACAAGGAAATCCTATTTG |
| | | TAAATCGTGTTGTGTTGGTGAATATTGCGACTAC |
| | | CTACGTAATTATAGACTTGATCCATTTCCTCCAT |
| | | GCAAACTATCTA |

### Example 9: Generation of a Modified Oncolytic Vaccinia Virus Expressing CXCR3, IL-12, and Tgfb-Inhibitor

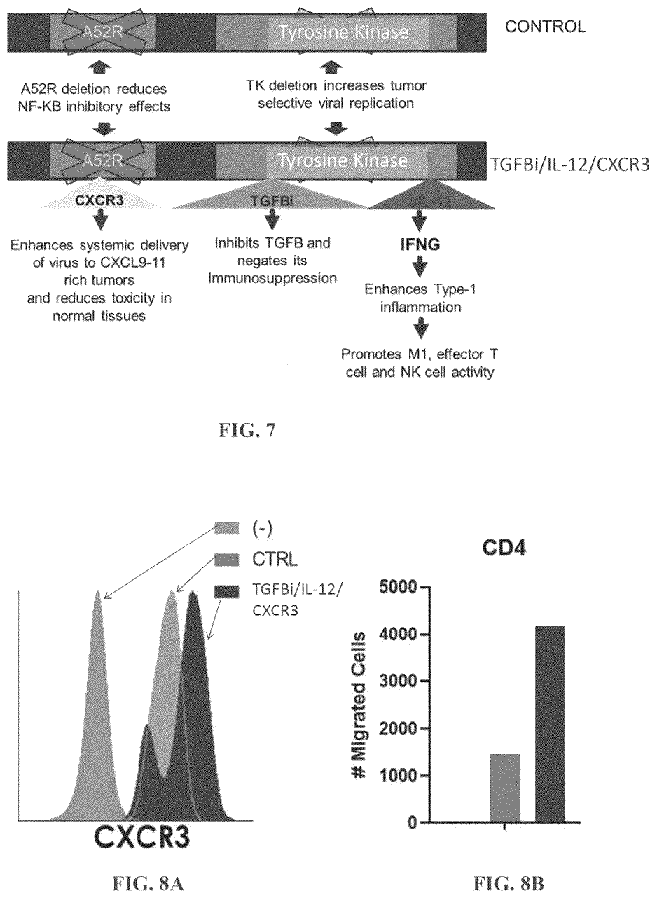

A modified oncolytic vaccinia virus expressing CXCR3 as described by SEQ ID NO: 43, IL-12 as described by SEQ ID NO: 15, and a TGFB1 antagonizing mini-monomer construct as described by SEQ ID NO: 41, as shown in FIG. 7 was generated according to methods described in Examples 7 and 8. Expression and function of the transgenes in the modified virus were confirmed as follows.

### CXCR3

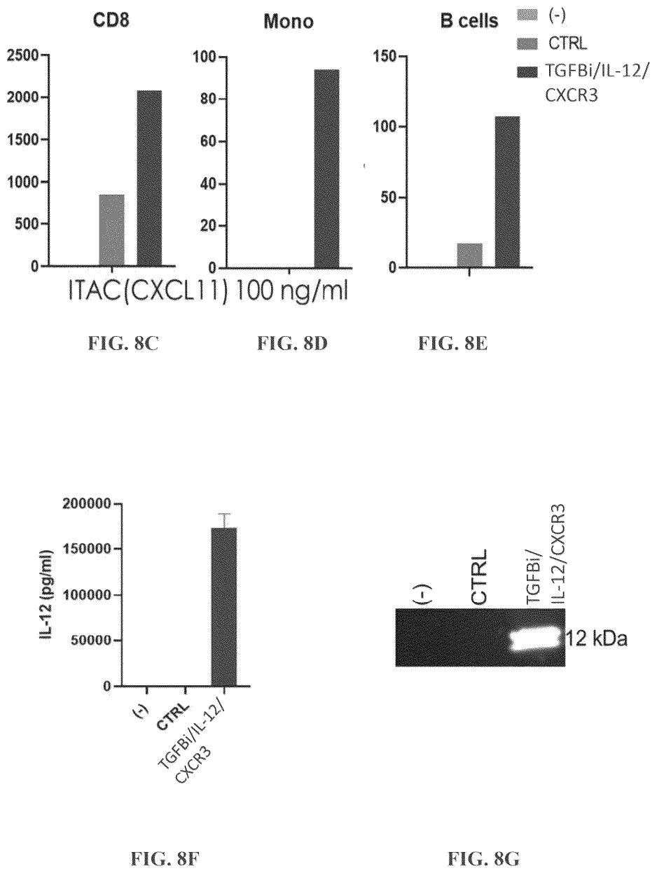

Fluorescent activated cell sorting (FACS) analysis of HeLa cells infected with modified virus, depicted in FIG. 8A, confirms expression of CXCR3. Function of expressed CXCR3 was tested in peripheral blood mononuclear cells (PBMC), including CD4+ cells, CD8+ cells, monocytes, and B cells. Subpopulations of PBMCs infected with modified virus were compared to TK-virus infected control (CTRL) or non-infected cells (−) cells for migration to 100 ng/ml ITAC (CXCL11), a ligand for CXCR3. Counts of migrated cells in each test group are shown as barplots in FIG. 8B-FIG. 8E. FIG. 8B shows more than 4-fold increase in migrated modified virus-infected CD4 cells compared to control cells. FIG. 8C shows an approximate 2.5-fold increase in migrated modified virus-infected CD8 cells compared to control cells. FIG. 8D shows migration of monocytes in infected cells, compared to no response in control cells. FIG. 8E shows an approximate 10-fold increase in migrated modified virus-infected B cells compared to control cells. The increased amount of migration to the CXCR3 ligand indicates expression of active form of the receptor in cells infected with the modified virus.

### IL-12

ELISA assay of culture supernatant from Hela cells infected with modified virus confirmed expression of IL-12 with conserved epitopes. FIG. 8F is a barplot showing quantitative ELISA results from supernatants of Hela cells infected with modified virus compared to TK-virus infected control (CTRL) or non-infected cells (−). Approximately 170,000 μg/ml IL-12 was detected in supernatants from modified virus-infected cells. No IL-12 was detected from untreated and negative control cells.

### TGFBi

Hela cell culture lysates from cells infected with modified virus, TK-virus infected control (CTRL) or non-infected cells (−) were analysed using Western Blot. As shown in FIG. 8G, lysate from modified virus infected cells showed a 12 kDa protein corresponding to the TGFBi protein. Untreated and negative control cultures did not show a corresponding band.

### Example 10: Modified Virus Rescues CD8 T Cells from Suppression by TGF-Beta and Induces More Granzyme B

Hela cells were treated with media (−) or infected with TK-control virus (CTRL) or modified virus (TGFBi/IL-12/CXCR3) at a multiplicity of infection (MOI) of 10 for 24 hours. Virus free supernatants from the infected cells were harvested and used for subsequent T cell experiments. CD8+ T cells treated with supernatants from Hela cells infected with modified virus displayed less suppression with treatment with TGF-beta 1 (TGFB1), indicating expression of functional TGFB inhibitor. CD8+ T cells were labelled with 2 mM CFSE and were stimulated with anti-CD3 and anti-CD28 antibodies in the presence of supernatants from Hela cells infected with modified virus, or with TK-control virus (CTRL) or uninfected cells. T cells were then exposed to 0 ng/ml, 10 ng/ml, or 50 ng/ml TGFB1.

T cells were analyzed for CD44 and GZMB expression. T cells were stained with antibodies for surface-expressed CD44, then fixed and permeabilized using a Cytofix/Cytoperm™ Fixation/Permeablization Kit (BD Biosciences, Franklin Lakes, NJ). Cells were stained with antibodies for cytoplasmic GZMB and analyzed using an Attune flow cytometer (Thermo Fisher Scientific, Carlsbad, CA).

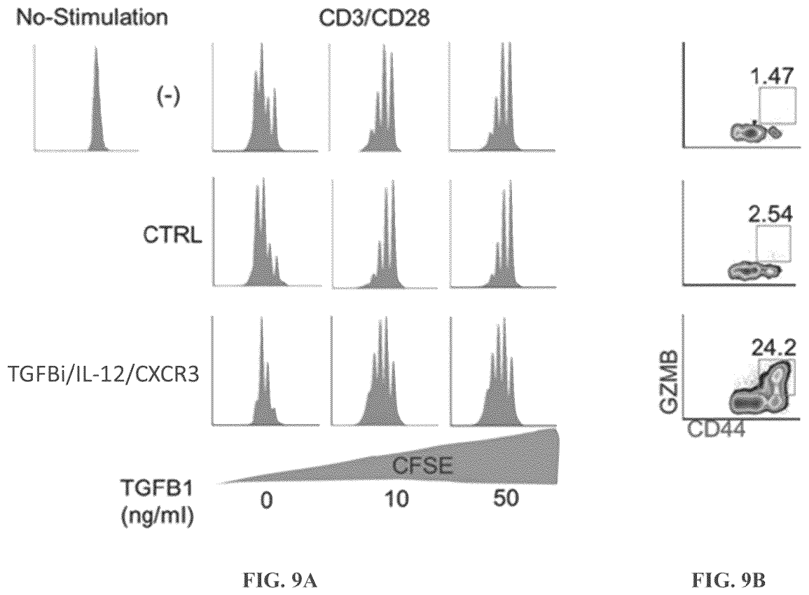

The initial population of cells with incorporated stain appears as a peak with the highest intensity in the trace. Subsequent generations of cells contain diluted amounts of stain and appear as peaks of diminishing fluorescence, or progressing to the left. Cells susceptible to TGFB1 suppression show smaller generational peaks in the trace. Traces depicted in FIG. 9A show a decrease in generational peaks, indicating suppression of proliferation, in TK-control virus (CTRL) infected and uninfected (−) CD8 T cells after exposure to 10 ng/ml and 50 ng/ml TGFB1. Stimulated T cells treated with modified virus showed less suppression of proliferation after contacting with TGFB1 compared to controls.

CD8 cells were also assessed for granzyme B (GZMB) induction. FCS files generated by Attune were analyzed using FCS 7 software. Gating was for live T cells. Density plots were made with CD44 in the X-axis and GZMB in the Y-axis. The percentage of GZMB was analyzed by gating of GZMB+ T cells in the density plot. FIG. 9B shows dot plots of each sample group. The plots show increased levels of CD44+ GZMB+ in CD8 T cells infected with modified virus compared to untreated (−) and TK-virus infected control cells.

### Example 11: In Vitro Selectivity and Enhanced In Vivo Tumor Specific Delivery of Modified Vaccinia Virus

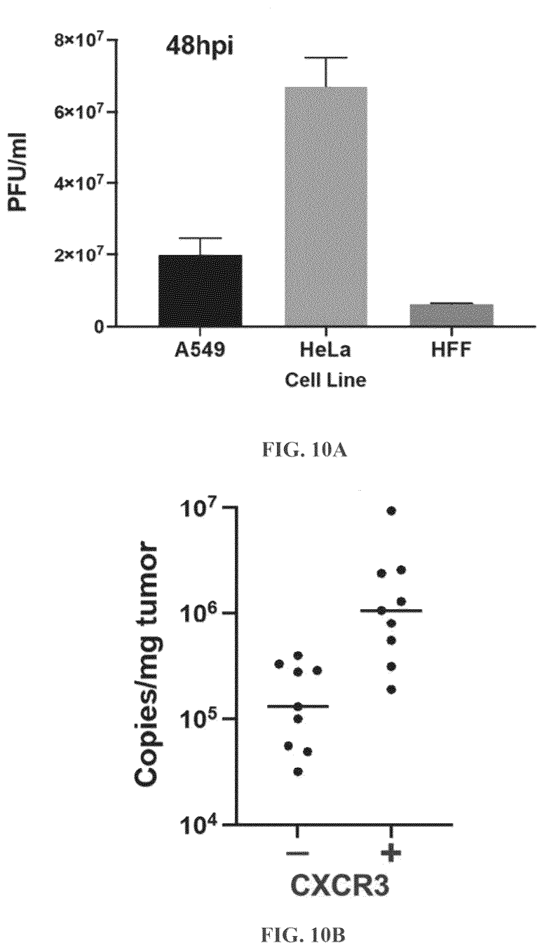

Using an in vivo mouse RENCA tumor model, the selectivity of the modified virus was tested with comparison to control virus. Modified virus as described in Example 9 was tested for infectivity in cancer and non-cancerous cell lines. Three cell types, human lung adenocarcinoma (A549) cells, human cervical cancer (Hela) cells, and Human Foreskin Fibroblasts (HFF) were analyzed for viral load 48 hours post infection. Detected PFU/ml in each cell type is depicted in bar graphs in FIG. 10A. More than 10-fold PFU/ml virus was detected in Hela cells 48 hours after infection compared to HFF cells. A549 cells showed an approximate 4-fold higher virus load as compared to infected HFF cells. The modified virus shows in vitro preferential infectivity in tumor cells.

BALB/c mice were implanted subcutaneously with Renca tumors. 1×10⁷ PFU modified virus with and without modification to express CXCR3 was administered intravenously to the mice. Tumors were recovered 24 hours later and viral genomes were quantified by qPCR. Results are shown in the scatter plot in FIG. 10B. Approximately 10-fold more viral genome was detected in Renca tumors in mice treated with the virus expressing CXCR3 than in tumors from mice treated with the virus without CXCR3 modification. Results showed the modified virus had increased in vivo infectivity compared to unmodified virus.

### Example 12: Modified Vaccinia Virus Decreases Tumor Burden and Increases Mice Survival in Multiple Tumor Models

Using in vivo mouse EMT-6 and MC38 tumor models, the therapeutic efficacy of the modified virus was tested with comparison to control virus and buffer negative control. Mice were seeded with EMT6 and MC38 tumors.

BalbC mice were implanted with 1×10⁵ EMT6 cells. Thirteen days after implantation, tumors reached an average volume of 80.26 mm³ (26.70-136.23 mm³). Mice with tumors less than 36.92 mm³ an larger than 117.43 mm³ were excluded and mice were sorted so that each treatment group (n=10) had an average tumor volume of ~78 mm³. Mice were treated with 1×10⁴, 1×10⁵, 1×10⁶ or 1×10⁷ PFU vaccinia virus modified as described in Example 9 (TGFBi/IL-12/CXCR3), buffer (−), or TK-control virus (CTRL). Treatment was via IV injection (tail-vein) on days 0 and 3. Tumor volumes were measured twice weekly and mice were weighed once weekly.

C57Bl/6 mice were implanted with 5×10⁵ cells and tumors were allowed to form over 8 days, reaching an average of 50.46 mm³ (10.17-84.79 mm³). Mice with tumors less than 31.99 mm³ were excluded and mice were sorted so that each group (n=10) have an average volume of 53.06 mm³. Mice were treated with 2×1×10⁵, 2×1×10⁶ or 1×1×10⁷ PFU/dose vaccinia virus modified as described in Example 9 (TGFBi/IL-12/CXCR3), buffer (−), or TK-control virus (CTRL). Treatment was via IV injection (tail-vein) on days 0 and 3. Tumors were measured twice weekly, and mice were weighed once weekly.

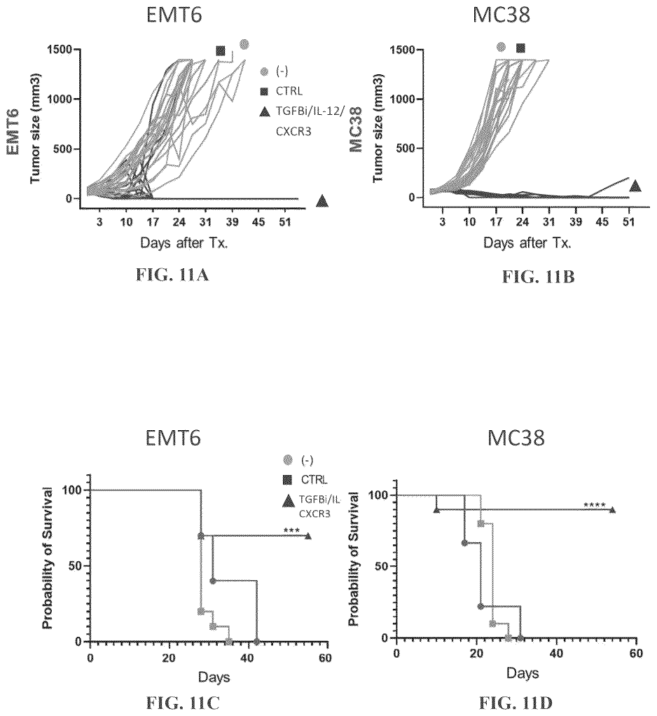

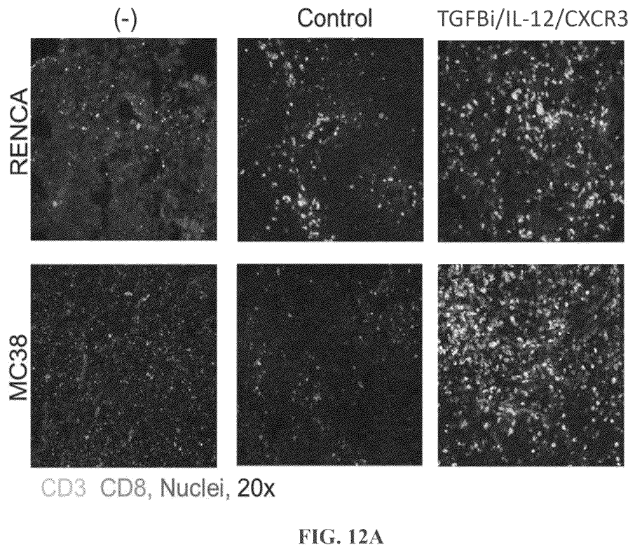

All buffer control (−) and control virus (CTRL) treated mice in both tumor models were euthanized by day 42. 8 of the 10 EMT6 mice treated with modified virus showed complete suppression of tumor growth to day 54. MC38 mice treated with modified virus showed complete suppression of tumor growth beyond 39 days, with complete suppression to Day 51 in 9 of 10 mice. Probability of Survival out to 54 days post treatment in EMT6 tumor mice and MC38 tumor mice is shown in FIG. 11C and FIG. 11D, respectively. Asterisks represent P values: *P≤0.05; **P≤0.01; ***P≤0.001; and ****P≤0.0001. As shown in FIG. 11C, mice treated with modified virus maintained 70% Probability of Survival to Day 54. As shown in FIG. 11D, mice treated with modified virus maintained 90% Probability of Survival to Day 54. Results indicate treatment with modified virus suppressed tumor growth in multiple tumor models and improved probability of survival.

### Example 13: Modified Virus Treatment Increases CD3+CD8+ T Cell Infiltrate in Tumors

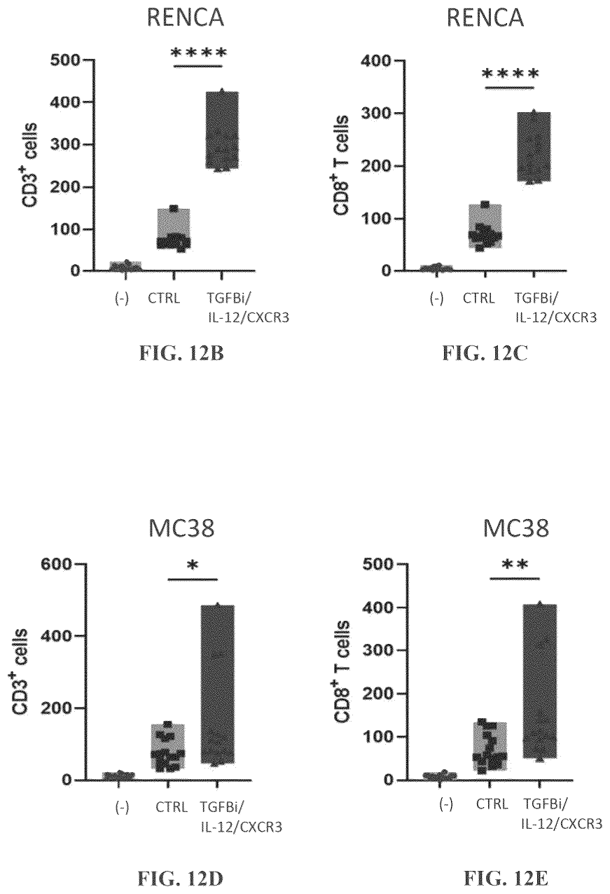

Post mortem analysis was used to analyze the impact of the modified virus on immune/stromal/endothelial milieu of the tumors and to determine toxicity profile. Renca tumors from Example 11 and MC38 tumors from Example 12 were sectioned and treated with nucleic, CD3 and CD8 stains. Representative stained sections are shown in FIG. 12A. Tumors treated with modified virus showed greater infiltration of CD3 and CD8 markers compared to corresponding sections from untreated or control samples, indicating infiltration of cytotoxic T cells.

FIG. 12B-FIG. 12E are barplots of total counts of CD3+ and CD8+ T cells in RENCA and MC38 tumor samples following treatment with modified virus or untreated or control samples. FIG. 12B shows an approximate 3-fold increase in CD3+ T cells in RENCA tumors treated with modified virus compared to control cells. FIG. 12C shows an approximate 3.5-fold increase in CD8+ T cells in RENCA tumors treated with modified virus compared to control cells. FIG. 12D shows an approximate 2-fold increase in mean number of CD3+ T cells after treatment with modified virus compared to control cells. FIG. 12E shows an approximate 2-3-fold increase in mean number of CD8+ T cells after treatment with modified virus compared to control cells.

### Example 14: Modified Virus Induces Type II Interferon-G Associated but Negates TGFB1 Associated Gene Signatures in Tumors

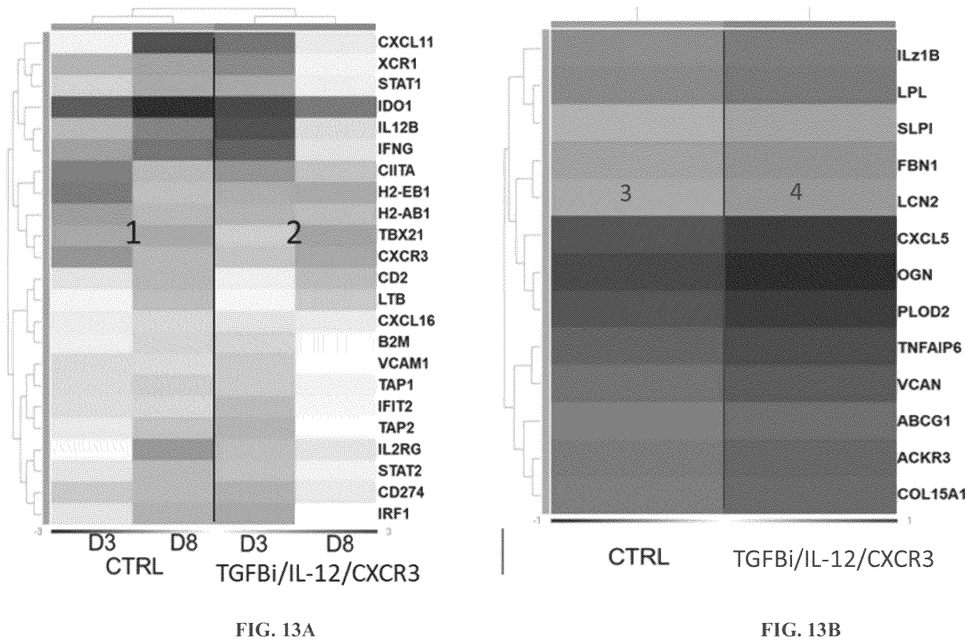

Whole RENCA tumors from treated mice described in Example 11 were harvested and homogenized in Qiazol (Qiagen, Hilden, Germany) using a bead mill. RNA was extracted using Rneasy Kit (Qiagen). Extracted RNA was sequenced using next-generation sequencing methods. Raw RNA seq data (FASTQ) files were analyzed for differential gene expression between TK-control virus (CTRL) and modified virus using Rosalind software, and gene expression heatmaps were generated, as shown in FIG. 13A and FIG. 13B.

FIG. 13A is a heatmap showing a relative expression levels of Type II interferon-gamma (INFG)-associated genes, specifically compared to an overall mean. Expression levels were tested for CXCL11, XCR1, STAT1, IDO1, IL12B, IFNG, CIITA, H2-EB1, H2-AB1, TBX21, CXCR3, CD2, LTB, CXCL16, B2M, VCAM1, TAP1, IFIT2, TAP2, IL2RG, STAT2, CD274, IRF1. Area 1 shows control CD3+ and CD8+ cells exhibit generally lower expression levels compared to an overall mean, while area 2 shows CD3+ and CD8+ cells treated with modified virus exhibit higher levels of expression. This shows contacting cells with a modified virus expressing TGF-beta inhibitor activates expression of IFNG-associated genes.

FIG. 13B is a heatmap showing relative expression levels of TGF-beta1-associated genes compared to an overall mean. Expression levels were tested for ILz1B, LPL, SLP1, FBN1, LCN2, CXCL5, OGN, PLOD2, TNFAIP6, CAN, ABCG1, ACKR3, COL15A1. Area 3 shows control cells exhibit generally higher expression levels compared to an overall mean, while area 4 shows cells treated with modified virus exhibit lower levels of expression. This shows contacting cells with a modified virus expressing a TGF-beta inhibitor reduces expression in TGF-beta1 associated genes.

The foregoing description and accompanying drawings set forth a number of representative embodiments at the present time. Various modifications, additions, and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A composition, wherein the composition comprises:
a vector, wherein the vector comprises:
an exogenous nucleic acid comprising a sequence encoding for a cytokine or a functional variant thereof;
an exogenous nucleic acid comprising a sequence encoding for a chemokine receptor or a functional variant thereof; and
a first promoter region, wherein the first promoter region is upstream to the sequence encoding for the chemokine receptor and provides for expression of the chemokine receptor prior to expression of the cytokine.

2. The composition of claim 1, wherein the encoded chemokine receptor comprises at least one of: a CXC receptor, a CC receptor, a CX3C receptor, an XC receptor, a functional fragment thereof, a functional variant thereof, or any combinations thereof.

3. The composition of claim 1, wherein the encoded chemokine receptor comprises at least one of: CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, a functional fragment thereof or a functional variant thereof, or any combinations thereof.

4. The composition of claim 1, wherein the encoded chemokine receptor comprises CXCR3, and wherein the encoded chemokine receptor comprises an amino acid sequence having at least 85%, 90%, 95%, 99%, or 100% sequence identity to SEQ ID NO: 43 or SEQ ID NO: 42.

5. The composition of claim 1, wherein the first promoter region comprises an early promoter, and wherein the early promoter comprises any one of A52R, pB8, mH5, I4L, LEO, pF11, I3L, P7.5, TK promoter, F7L, H5R, a short synthetic promoter (SSP) or any variation or combination thereof.

6. The composition of claim 5, wherein the early promoter comprises the A52R promoter.

7. The composition of claim 1, wherein the encoded cytokine comprises IL-12 or a functional variant thereof.

8. The composition of claim 7, wherein the encoded IL-12 comprises an amino acid sequence having at least 85%, 90%, 95%, 99%, or 100% sequence identity to either one of SEQ ID NO: 15 or SEQ ID NO: 12.

9. The composition of claim 7, wherein the sequence encoding for the IL-12 comprises a nucleic acid sequence having at least 85%, 90%, 95%, 99%, or 100% sequence identity to either one of SEQ ID NO: 66 or SEQ ID NO: 64.

10. The composition of claim 1, further comprising an exogenous nucleic acid comprising a sequence encoding for a Transforming growth factor beta (TGF-beta) activity inhibitor; wherein the first promoter region provides for expression of the chemokine receptor prior to expression of the TGF-beta activity inhibitor.

11. The composition of claim 10, wherein the encoded TGF-beta activity inhibitor comprises a TGF-beta dominant negative, a TGF-beta receptor dominant negative, a protein that binds TGF-beta, or a protein that binds to a TGF-beta receptor.

12. The composition of claim 11, wherein the encoded protein that binds TGF-beta receptor is a protein comprising a domain of TGF-beta, and wherein the encoded protein comprising a domain of TGF-beta comprises an amino acid sequence as set forth in any one of SEQ ID NOs: 1-9.

13. The composition of claim 10, wherein the sequence encoding for the Transforming growth factor beta (TGF-beta) activity inhibitor comprises a nucleic acid sequence having at least 85%, 90%, 95%, 99%, or 100% sequence identity to SEQ ID NO: 67.

14. The composition of claim 11, wherein the encoded protein that binds to TGF-beta receptor is a TGF-beta fusion protein, and wherein the TGF-beta fusion protein comprises a human IgE signal peptide and a TGF-beta variant 2 (TGFbv2).

15. The composition of claim 14, wherein the human IgE signal peptide comprises an amino acid sequence having at least 85%, 90%, 95%, 99%, or 100% sequence identity to SEQ ID NO: 37.

16. The composition of claim 14, wherein the TGFbv2 comprises an amino acid sequence having at least 85%, 90%, 95%, 99%, or 100% sequence identity to SEQ ID NO: 8.

17. The composition of claim 10, wherein the sequence encoding for the cytokine comprises a second promoter region that provides for expression of the cytokine, and wherein the sequence encoding for the TGF-beta activity inhibitor comprises a third promoter region that provides for expression of the TGF-beta activity inhibitor, and wherein each of the second and third promoter regions comprise a late promoter comprising any one of SSP, P7.5, P28, P135, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof.

18. The composition of claim 17, wherein the second promoter region comprises a P135 promoter, and wherein the third promoter region comprises a P7.5 promoter.

19. The composition of claim 10, wherein the exogenous nucleic acid encoding for a cytokine or a functional variant thereof, the exogenous nucleic acid encoding for the TGF-beta activity inhibitor, and the exogenous nucleic acid encoding for the chemokine receptor or functional variant thereof are located on a single genome.

20. The composition of claim 1, wherein the vector is an oncolytic virus, and wherein the oncolytic virus comprises at least one genome modification.

21. The composition of claim 20, wherein the at least one modification comprises mutation or deletion of at least one gene selected from the group consisting of: Thymidine Kinase (TK)21, F13L, A36R, A34R, A33R, A52R, B5R, B8R, B18R, SPI-1, SPI-2, B15R, VGF, E3L, K3L, A41L, K7R, or N1L, a functional fragment thereof, or any combination thereof.

22. The composition of claim 21, wherein the at least one modification comprises a deletion of genes A52R and TK.

23. The composition of claim 20, wherein the oncolytic virus is a poxvirus, an adeno associated virus, an adenovirus, a reovirus, a lentivirus, a herpes simplex virus, a vesicular stomatitis virus, a mengovirus, a myxoma virus, Newcastle disease virus, measles virus, or polio virus.

24. The composition of claim 23, wherein the poxvirus is a vaccinia virus, and wherein the vaccinia virus is a Western Reserve strain.

25. A nucleic acid molecule, wherein the nucleic acid molecule comprises:
an insertion at an A52R locus comprising a sequence as set forth in SEQ ID NO: 88;
an insertion at a TK gene locus comprising a sequence as set forth in SEQ ID NO: 85.

26. A pharmaceutical composition, wherein the pharmaceutical composition comprises:
the composition of claim 1; and
a pharmaceutically acceptable excipient.

27. A method for treatment of cancer comprising administering to a subject having cancer the pharmaceutical composition of claim 26 in an amount sufficient for treatment of a cancer.


SEQUENCE LISTING (88 sequences)

SEQ ID NO: 1 - protein, 113 aa, Homo sapiens
MALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 113 aa, Homo sapiens
MALDAAYCFRNVQDNCCLRPLYIDFKRDLGWKWIHEPKGYNANFCAGACPYLWSSDTQHTKVLSLYNTINPEASASPCCVSQDLEPLTILYYIGKTPKIEQLSNMIVKSCKCS

SEQ ID NO: 3 - protein, 113 aa, Homo sapiens
MALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 4 - protein, 93 aa, synthetic construct
MALDTNYCFSSTEKNCCVRQLYIDFRKDLGWKWIHEPKGYHANFCLGPCPYRASKSPSCVPQALEPLPIVYYVGRKPKVEQLSNMIVRSCKCS

SEQ ID NO: 5 - protein, 93 aa, synthetic construct
MALDAAYCFRNVQDNCCLRPLYIDFKRDLGWKWIHEPKGYNANFCAGACPYRASKSPSCVSQDLEPLTILYYIGKTPKIEQLSNMIVKSCKCS

SEQ ID NO: 6 - protein, 93 aa, synthetic construct
FALDAAYCFRNVQDNCCLRPLYIDFRKDLGWKWIHEPKGYNANFCAGACPYRASKSPSCVSQDLEPLTIVYYVGRKPKVEQLSNMIVKSCKCS

SEQ ID NO: 7 - protein, 92 aa, synthetic construct
ALDAAYVFRNVQDNCALRPLYIDFRKDLGWKWIHEPKGYNANFCAGACPYRASKSPRCRSQDLEPLTIVYYVGRKPKVEQLSNMIVKSCKCS

SEQ ID NO: 8 - protein, 115 aa, synthetic construct
KEVLASSQEALVVTERKYLKSDWCKLRPLYIDFRKDLGWKWIHEPKGYNANFCYGQCNSFYIPRHVKKEEDSFQSSAFCVSQDLEPLTIVYYVGRKPKVEQLSNMIVKSCRCMSV

SEQ ID NO: 9 - protein, 93 aa, synthetic construct
MALDTNYCFRNLEENCCVRPLYIDFRQDLGWKWVHEPKGYYANFCSGPCPYEESDSPSCVPQDLEPLTILYYVGRTPKVEQLSNMVVKSCKCS

SEQ ID NO: 10 - protein, 15 aa, synthetic construct
KRIWFIPRSSWYERA

SEQ ID NO: 11 - protein, 14 aa, synthetic construct
TSLDASIIWAMMQN

SEQ ID NO: 12 - protein, 562 aa, Mus sp.
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSGGGGSASATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 13 - protein, 215 aa, Mus sp.
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 14 - protein, 335 aa, Mus sp.
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 15 - protein, 602 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSASWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 16 - protein, 252 aa, Homo sapiens
WPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 17 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 18 - protein, 22 aa, synthetic construct
GGGGSGGGGSGGGGSGGGGSAS

SEQ ID NO: 19 - protein, 15 aa, synthetic construct
  VARIANT 1..15: note = This sequence may encompass 1-3 GGGGS repeating units
GGGGSGGGGSGGGGS

SEQ ID NO: 20 - protein, 8 aa, synthetic construct
  VARIANT 1..8: note = This sequence may encompass 6-8 residues
GGGGGGGG

SEQ ID NO: 21 - protein, 15 aa, synthetic construct
  VARIANT 1..15: note = This sequence may encompass 1-3 EAAAK repeating units
EAAAKEAAAKEAAAK

SEQ ID NO: 22 - protein, 46 aa, synthetic construct
AEAAAKEAAAKEAAAKEAAAKALEAEAAAKEAAAKEAAAKEAAAKA

SEQ ID NO: 23 - protein, 5 aa, synthetic construct
PAPAP

SEQ ID NO: 24 - skipped

SEQ ID NO: 25 - protein, 17 aa, synthetic construct
VSQTSKLTRAETVFPDV

SEQ ID NO: 26 - protein, 6 aa, synthetic construct
PLGLWA

SEQ ID NO: 27 - protein, 6 aa, synthetic construct
RVLAEA

SEQ ID NO: 28 - protein, 10 aa, synthetic construct
EDVVCCSMSY

SEQ ID NO: 29 - protein, 8 aa, synthetic construct
GGIEGRGS

SEQ ID NO: 30 - protein, 10 aa, synthetic construct
TRHRQPRGWE

SEQ ID NO: 31 - protein, 10 aa, synthetic construct
AGNRVRRSVG

SEQ ID NO: 32 - protein, 9 aa, synthetic construct
RRRRRRRRR

SEQ ID NO: 33 - protein, 4 aa, synthetic construct
GFLG

SEQ ID NO: 34 - skipped

SEQ ID NO: 35 - protein, 17 aa, synthetic construct
GGGGSSPLGLWAGGGGS

SEQ ID NO: 36 - protein, 20 aa, Mus sp.
MYSMQLASCVTLTLVLLVNS

SEQ ID NO: 37 - protein, 20 aa, Homo sapiens
MDWTWILFLVAAATRVHSGR

SEQ ID NO: 38 - protein, 48 aa, unidentified
MRISKPHLRSISIQCYLCLLLNSHFLTEAGIHVFILGCFSAGLPKTEA

SEQ ID NO: 39 - protein, 21 aa, unidentified
MVLGTIDLCSCFSAGLPKTEA

SEQ ID NO: 40 - protein, 112 aa, synthetic construct
MYSMQLASCVTLTLVLLVNSALDAAYVFRNVQDNCALRPLYIDFRKDLGWKWIHEPKGYNANFCAGACPYRASKSPRCRSQDLEPLTIVYYVGRKPKVEQLSNMIVKSCKCS

SEQ ID NO: 41 - protein, 135 aa, synthetic construct
MDWTWILFLVAAATRVHSGRKEVLASSQEALVVTERKYLKSDWCKLRPLYIDFRKDLGWKWIHEPKGYNANFCYGQCNSFYIPRHVKKEEDSFQSSAFCVSQDLEPLTIVYYVGRKPKVEQLSNMIVKSCRCMSV

SEQ ID NO: 42 - protein, 367 aa, Mus sp.
MYLEVSERQVLDASDFAFLLENSTSPYDYGENESDFSDSPPCPQDFSLNFDRTFLPALYSLLFLLGLLGNGAVAAVLLSQRTALSSTDTFLLHLAVADVLLVLTLPLWAVDAAVQWVFGPGLCKVAGALFNINFYAGAFLLACISFDRYLSIVHATQIYRRDPRVRVALTCIVVWGLCLLFALPDFIYLSANYDQRLNATHCQYNFPQVGRTALRVLQLVAGFLLPLLVMAYCYAHILAVLLVSRGQRRFRAMRLVVVVVAAFAVCWTPYHLVVLVDILMDVGVLARNCGRESHVDVAKSVTSGMGYMHCCLNPLLYAFVGVKFREQMWMLFTRLGRSDQRGPQRQPSSSRRESSWSETTEASYLGL

SEQ ID NO: 43 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 44 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 45 - protein, 374 aa, unidentified; identical to SEQ ID NO: 44

SEQ ID NO: 46 - protein, 357 aa, Homo sapiens
MEDFNMESDSFEDFWKGEDLSNYSYSSTLPPFLLDAAPCEPESLEINKYFWIIYALVFLLSLLGNSLVMLVILYSRVGRSVTDVYLLNLALADLLFALTLPIWAASKVNGWIFGTFLCKWSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAWLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 47 - protein, 374 aa, Homo sapiens; identical to SEQ ID NO: 44

SEQ ID NO: 48 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVAVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 49 - protein, 359 aa, Mus sp.
MEPISVSIYTSDNYSEEVGSGDYDSNKEPCFRDENVHFNRIFLPTIYFIIFLTGIVGNGLVILVMGYQKKLRSMTDKYRLHLSVADLLFVITLPFWAVDAMADWYFGKFLCKAVHIIYTVNLYSSVLILAFISLDRYLAIVHATNSQRPRKLLAEKAVAVGVWIPALLLTIPDFIFADVSQGDISQGDDRYICDRLYPDSLWMVVFQFQHIMVGLVLPGIVILSCYCIIISKLSHSKGHQKRKALKTTVILILAFFACWLPYYVGISIDSFILLGVIKQGCDFESIVHKWISITEALAFFHCCLNPILYAFLGAKFKSSAQHALNSMSRGSSLKILSKGKRGGHSSVSTESESSSFHSS

SEQ ID NO: 50 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 51 - protein, 352 aa, Homo sapiens; identical to SEQ ID NO: 48

SEQ ID NO: 52 - protein, 359 aa, Mus sp.; identical to SEQ ID NO: 49

SEQ ID NO: 53 - protein, 352 aa, Homo sapiens; identical to SEQ ID NO: 50

SEQ ID NO: 54 - protein, 90 aa, Homo sapiens
MKVSAAALAVILIATALCAPASASPYSSDTTPCCFAYIARPLPRAHIKEYFYTSGKCSNPAWFVTRKNRQVCANPEKKWVREYINSLEMS

SEQ ID NO: 55 - protein, 349 aa, Homo sapiens
MDYQVSSPIYDINYYTSEPCQKINVKQIAARLLPPLYSLVFIFGFVGNMLVILILINCKRLKSMTDIYLLNLAISDLFFLLTVPFWAHYAAAQWDFGNTMCQLLTGLYFIGFFSGIFFIILLTIDRYLAWHAVFALKARTVTFGWTSVITWWAVFASLPGIIFTRSQKEGLHYTCSSHFPYSQYQFWKNFQTLKIVILGLVLPLLVMVICYSGILKTLLRCRNEKKRHRAVRLIFTIMIVYFLFWAPYNIVLLLNTFQEFFGLNNCSSSNRLDQAMQVTETLGMTHCCINPIIYAFVGEKFRNYLLVFFQKHIAKRFCKCCSIFQQEAPERASSVYTRSTGEQEISVGL

SEQ ID NO: 56 - DNA, 29 bp, Vaccinia virus
  source 1..29: mol_type = genomic DNA
gcgtcctaaaattaaaacaaatgatataa

SEQ ID NO: 57 - DNA, 130 bp, Vaccinia virus
  source 1..130: mol_type = genomic DNA
tcactaattccaaacccacccgctttttatagtaagtttttcacccataaataataaatacaataattaatttctcgtaaaagtagaaaatatattctaatttattgcacggtaaggaagtagatcataa

SEQ ID NO: 58 - DNA, 37 bp, unidentified
  source 1..37: mol_type = unassigned DNA
aagctcacaaaaaaaacttctctaaatagattcgagc

SEQ ID NO: 59 - DNA, 204 bp, Vaccinia virus
  source 1..204: mol_type = genomic DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 60 - DNA, 60 bp, unidentified
  source 1..60: mol_type = unassigned DNA
agataaagtgaaaatatatatcattatattacaaagtacaattatttaggtttaatcaga

SEQ ID NO: 61 - DNA, 52 bp, unidentified
  source 1..52: mol_type = unassigned DNA
aatccctattaatgaaaagttaaataatttttttattacaccaacaaaaamt

SEQ ID NO: 62 - DNA, 64 bp, unidentified
  source 1..64: mol_type = unassigned DNA
ggatcataaaaatatagtagaatttcattttgtttttttctatgctataaatagcttctcgagc

SEQ ID NO: 63 - DNA, 511 bp, Homo sapiens
  source 1..511: mol_type = genomic DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 64 - DNA, 1689 bp, Mus sp.
  source 1..1689: mol_type = genomic DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccatgcagggtccgatccggaggtggcggatccggcggaggtggctcaggtggcggtggatcaggaggaggtggatcagctagcgccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgaccatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgacgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagttccgcctaa

SEQ ID NO: 65 - DNA, 339 bp, synthetic construct
  source 1..339: mol_type = other DNA
atgtattctatgcaactggcatcatgcgttacccttaccttggtgcttctcgtcaatagcgctctggacgctgcttacgtcttccgtaacgttcaggacaactgcgctctgcgtccgctgtacatcgacttccgcaaagacctgggttggaaatggatccacgaaccgaaaggttacaacgctaacttctgcgctggtgcttgcccgtaccgtgcttccaaatccccgcgctgccgatcccaggacctggaaccgctgaccatcgtgtactacgtgggtcgcaaaccgaaagtggaacagctgtccaacatgatcgttaaatcctgcaaatgctcctag

SEQ ID NO: 66 - DNA, 1809 bp, Homo sapiens
  source 1..1809: mol_type = genomic DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggaggtggcggatccggcggaggtggctcaggtggcggtggatcaggaggaggtggatcagctagctggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 67 - DNA, 408 bp, synthetic construct
  source 1..408: mol_type = other DNA
atggactggacatggattctctttctagtggccgcagccacaagggtccacagcggccgcaaggaagtccttgcttctagtcaggaagcgctcgttgtaacagaaagaaaatatctcaaatccgactggtgtaagctgcgaccactctacatagattttcgaaaggacctgggctggaaatggatacatgagccaaagggctataatgcgaatttttgctacggacaatgtaattctttctatatcccccggcacgtcaaaaaggaggaagattctttccaatcatctgcattctgtgtaagccaggatctcgagccgttgactattgtctactacgttggaagaaaaccgaaggtcgagcaactgtctaacatgatcgtcaaatcatgcaggtgcatgtcagtgtga

SEQ ID NO: 68 - DNA, 700 bp, synthetic construct
  source 1..700: mol_type = other DNA
ctcatcagacatatcgtgtgcatgcgtggagttaaattcatatcacgtagccattcttcccatggatgtttccttttttaccaaaggaaatgcatcattgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacgattaacgatgagtgtagatgctgttattttgaaccacagattaggattcttgatagagatgagatgctcaatggatcatcgtgtgatatgaacagacattgtattatgatgaatttacctgatgtaggcaaatttggatctagtatgttggggaaatatgaacctgacatgattaagattgctctttcggtggctggtaatttaataagaaatcgagactacattcccgggagacgaggatatagctactacgtttacggtatagcctctagataatttttttaagcacgaaataaaaaacataattttaaaccaatctatttcatactattttgtgtgatcacc

SEQ ID NO: 69 - DNA, 1104 bp, synthetic construct
  source 1..1104: mol_type = other DNA
atgtaccttgaggttagtgaacgtcaagtgctagatgcctcggactttgcctttcttctggaaaacagcacctctccctacgattatggggaaaacgagagcgacttctctgactccccgccctgcccacaggatttcagcctgaactttgacagaaccttcctgccagccctctacagcctcctcttcttgctggggctgctaggcaatggggcggtggctgctgtgctactgagtcagcgcactgccctgagcagcacggacaccttcctgctccacctggctgtagccgatgttctgctggtgttaactcttccattgtgggcagtggatgctgctgtccagtgggttttcggccctggcctctgcaaagtggcaggcgccttgttcaacatcaacttctatgcaggggccttcctgctggcttgtataagcttcgacagatatctgagcatagtgcacgccacccagatctaccgcagggacccccgggtacgtgtagccctcacctgcatagttgtatggggtctctgtctgctctttgccctcccagatttcatctacctatcagccaactacgatcagcgcctcaatgccacccattgccagtacaacttcccacaggtgggtcgcactgctctgcgtgtactgcagctagtggctggtttcctgctgccccttctggtcatggcctactgctatgcccatatcctagctgttctgctggtctccagaggccagaggcgttttcgagctatgaggctagtggtagtggtggtggcagcctttgctgtctgctggaccccctatcacctggtggtgctagtggatatcctcatggatgtgggagttttggcccgcaactgtggtcgagaaagccacgtggatgtggccaagtcagtcacctcgggcatggggtacatgcactgctgcctcaatccgctgctctatgcctttgtgggagtgaagttcagagagcaaatgtggatgttgttcacgcgcctgggccgctctgaccagagagggccccagcggcagccgtcatcttcacggagagaatcatcctggtctgagacaactgaggcctcctacctgggcttgtaa

SEQ ID NO: 70 - DNA, 13 bp, synthetic construct
  source 1..13: mol_type = other DNA
taggagctctaga

SEQ ID NO: 71 - DNA, 34 bp, synthetic construct
  source 1..34: mol_type = other DNA
ataacttcgtatagcatacattatacgaagttat

SEQ ID NO: 72 - DNA, 1399 bp, synthetic construct
  source 1..1399: mol_type = other DNA
aattctgcagatggatcataaaaatatagtagaatttcattttgtttttttctatgctataaatagcttctcgagcatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtcccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggaggctcaggaaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctga

SEQ ID NO: 73 - DNA, 18 bp, synthetic construct
  source 1..18: mol_type = other DNA
ttaattaaggccgctcga

SEQ ID NO: 74 - DNA, 500 bp, synthetic construct
  source 1..500: mol_type = other DNA
aaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaat

SEQ ID NO: 75 - DNA, 2351 bp, synthetic construct
  source 1..2351: mol_type = other DNA
ctcatcagacatatcgtgtgcatgcgtggagttaaattcatatcacgtagccattcttcccatggatgtttccttttttaccaaaggaaatgcatcattgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacgattaacgatgagtgtagatgctgttattttgaaccacagattaggattcttgatagagatgagatgctcaatggatcatcgtgtgatatgaacagacattgtattatgatgaatttacctgatgtaggcaaatttggatctagtatgttggggaaatatgaacctgacatgattaagattgctctttcggtggctggtaatttaataagaaatcgagactacattcccgggagacgaggatatagctactacgtttacggtatagcctctagataatttttttaagcacgaaataaaaaacataattttaaaccaatctatttcatactattttgtgtgatcaccatgtaccttgaggttagtgaacgtcaagtgctagatgcctcggactttgcctttcttctggaaaacagcacctctccctacgattatggggaaaacgagagcgacttctctgactccccgccctgcccacaggatttcagcctgaactttgacagaaccttcctgccagccctctacagcctcctcttcttgctggggctgctaggcaatggggcggtggctgctgtgctactgagtcagcgcactgccctgagcagcacggacaccttcctgctccacctggctgtagccgatgttctgctggtgttaactcttccattgtgggcagtggatgctgctgtccagtgggttttcggccctggcctctgcaaagtggcaggcgccttgttcaacatcaacttctatgcaggggccttcctgctggcttgtataagcttcgacagatatctgagcatagtgcacgccacccagatctaccgcagggacccccgggtacgtgtagccctcacctgcatagttgtatggggtctctgtctgctctttgccctcccagatttcatctacctatcagccaactacgatcagcgcctcaatgccacccattgccagtacaacttcccacaggtgggtcgcactgctctgcgtgtactgcagctagtggctggtttcctgctgccccttctggtcatggcctactgctatgcccatatcctagctgttctgctggtctccagaggccagaggcgttttcgagctatgaggctagtggtagtggtggtggcagcctttgctgtctgctggaccccctatcacctggtggtgctagtggatatcctcatggatgtgggagttttggcccgcaactgtggtcgagaaagccacgtggatgtggccaagtcagtcacctcgggcatggggtacatgcactgctgcctcaatccgctgctctatgcctttgtgggagtgaagttcagagagcaaatgtggatgttgttcacgcgcctgggccgctctgaccagagagggccccagcggcagccgtcatcttcacggagagaatcatcctggtctgagacaactgaggcctcctacctgggcttgtaataggagctctagaataacttcgtatagcatacattatacgaagttataaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaat

SEQ ID NO: 76 - DNA, 1000 bp, synthetic construct
  source 1..1000: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatc

SEQ ID NO: 77 - DNA, 37 bp, synthetic construct
  source 1..37: mol_type = other DNA
cctgcagggcgtcctaaaattaaaacaaatgatataa

SEQ ID NO: 78 - DNA, 19 bp, synthetic construct
  source 1..19: mol_type = other DNA
gtcgactgagtgaccgtga

SEQ ID NO: 79 - DNA, 136 bp, synthetic construct
  source 1..136: mol_type = other DNA
tcactaattccaaacccacccgctttttatagtaagtttttcacccataaataataaatacaataattaatttctcgtaaaagtagaaaatatattctaatttattgcacggtaaggaagtagatcataaggtacc

SEQ ID NO: 80-81 - skipped (2 entries)

SEQ ID NO: 82 - DNA, 20 bp, synthetic construct
  source 1..20: mol_type = other DNA
ttaattaaaaggccgctcga

SEQ ID NO: 83 - DNA, 997 bp, synthetic construct
  source 1..997: mol_type = other DNA
tattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 84 - DNA, 4326 bp, synthetic construct
  source 1..4326: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatccctgcagggcgtcctaaaattaaaacaaatgatataaatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccatgcagggtccgatccggaggtggcggatccggcggaggtggctcaggtggcggtggatcaggaggaggtggatcagctagcgccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgaccatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgacgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagttccgcctaagtcgactgagtgaccgtgatcactaattccaaacccacccgctttttatagtaagtttttcacccataaataataaatacaataattaatttctcgtaaaagtagaaaatatattctaatttattgcacggtaaggaagtagatcataaggtaccatggactggacatggattctctttctagtggccgcagccacaagggtccacagcggccgcaaggaagtccttgcttctagtcaggaagcgctcgttgtaacagaaagaaaatatctcaaatccgactggtgtaagctgcgaccactctacatagattttcgaaaggacctgggctggaaatggatacatgagccaaagggctataatgcgaatttttgctacggacaatgtaattctttctatatcccccggcacgtcaaaaaggaggaagattctttccaatcatctgcattctgtgtaagccaggatctcgagccgttgactattgtctactacgttggaagaaaaccgaaggtcgagcaactgtctaacatgatcgtcaaatcatgcaggtgcatgtcagtgtgagagctcataacttcgtatagcatacattatacgaagttattattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 85 - DNA, 4446 bp, synthetic construct
  source 1..4446: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattcttttttgtcatccctgcagggcgtcctaaaattaaaacaaatgatataaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggaggtggcggatccggcggaggtggctcaggtggcggtggatcaggaggaggtggatcagctagctggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaagtcgactgagtgaccgtgatcactaattccaaacccacccgctttttatagtaagtttttcacccataaataataaatacaataattaatttctcgtaaaagtagaaaatatattctaatttattgcacggtaaggaagtagatcataaggtaccatggactggacatggattctctttctagtggccgcagccacaagggtccacagcggccgcaaggaagtccttgcttctagtcaggaagcgctcgttgtaacagaaagaaaatatctcaaatccgactggtgtaagctgcgaccactctacatagattttcgaaaggacctgggctggaaatggatacatgagccaaagggctataatgcgaatttttgctacggacaatgtaattctttctatatcccccggcacgtcaaaaaggaggaagattctttccaatcatctgcattctgtgtaagccaggatctcgagccgttgactattgtctactacgttggaagaaaaccgaaggtcgagcaactgtctaacatgatcgtcaaatcatgcaggtgcatgtcagtgtgagagctcataacttcgtatagcatacattatacgaagttattattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 86 - DNA, 1107 bp, synthetic construct
  source 1..1107: mol_type = other DNA
atggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtga

SEQ ID NO: 87 - DNA, 854 bp, synthetic construct
  source 1..854: mol_type = other DNA
aaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaatggtaacgataacacaaaatgtgaacgctgcccacctcatacatataccacaatcccaattattctaatggatgtcatcaatgtagaaaatgcccaaccggatcatttgataaggtaaagtgtaccggaacacagaacagtaaatgttcgtgtcttcctggttggtattgtgctactgattcttcacagactgaagattgttgaaattgtgtaccaaaaaggagatgtccatgcggatactttggtggaatagatgaacaaggaaatcctatttgtaaatcgtgttgtgttggtgaatattgcgactacctacgtaattatagacttgatccatttcctccatgcaaactatcta

SEQ ID NO: 88 - DNA, 2661 bp, synthetic construct
  source 1..2661: mol_type = other DNA
ctcatcagacatatcgtgtgcatgcgtggagttaaattcatatcacgtagccattcttcccatggatgtttccttttttaccaaaggaaatgcatcattgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacgattaacgatgagtgtagatgctgttattttgaaccacagattaggattcttgatagagatgagatgctcaatggatcatcgtgtgatatgaacagacattgtattatgatgaatttacctgatgtaggcaaatttggatctagtatgttggggaaatatgaacctgacatgattaagattgctctttcggtggctggtaatttaataagaaatcgagactacattcccgggagacgaggatatagctactacgtttacggtatagcctctagataatttttttaagcacgaaataaaaaacataattttaaaccaatctatttcatactattttgtgtgatcaccatggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaaaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaatggtaacgataacacaaaatgtgaacgctgcccacctcatacatataccacaatcccaattattctaatggatgtcatcaatgtagaaaatgcccaaccggatcatttgataaggtaaagtgtaccggaacacagaacagtaaatgttcgtgtcttcctggttggtattgtgctactgattcttcacagactgaagattgttgaaattgtgtaccaaaaaggagatgtccatgcggatactttggtggaatagatgaacaaggaaatcctatttgtaaatcgtgttgtgttggtgaatattgcgactacctacgtaattatagacttgatccatttcctccatgcaaactatcta